SEQUENCE LISTING (81 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 1603 aa, Homo sapiens
MRVKPQGLVVTSSAVCSSPDYLREPKYYPGGPPTPRPLLPTRPPASPPDKAFSTHAFSENPRPPPRRDPSTRRPPVLAKGDDPLPPRAARPVSQARCPTPVGDGSSSRRCWDNGRVNLRPVVQLIDIMKDLTRLSQDLQHSGVHLDCGGLRLSRPPAPPPGDLQYSFFSSPSLANSIRSPEERATPHAKSERPSHPLYEPEPEPRDSPQPGQGHSPGATAAATGLPPEPEPDSTDYSELADADILSELASLTCPEAQLLEAQALEPPSPEPEPQLLDPQPRFLDPQALEPLGEALELPPLQPLADPLGLPGLALQALDTLPDSLESQLLDPQALDPLPKLLDVPGRRLEPQQPLGHCPLAEPLRLDLCSPHGPPGPEGHPKYALRRTDRPKILCRRRKAGRGRKADAGPEGRLLPLPMPTGLVAALAEPPPPPPPPPPALPGPGPVSVPELKPESSQTPVVSTRKGKCRGVRRMVVKMAKIPVSLGRRNKTTYKVSSLSSSLSVEGKELGLRVSAEPTPLLKMKNNGRNVVVVFPPGEMPIILKRKRGRPPKNLLLGPGKPKEPAVVAAEAATVAAATMAMPEVKKRRRRKQKLASPQPSYAADANDSKAEYSDVLAKLAFLNRQSQCAGRCSPPRCWTPSEPESVHQAPDTQSISHFLHRVQGFRRRGGKAGGFGGRGGGHAAKSARCSFSDFFEGIGKKKKVVAVAAAGVGGPGLTELGHPRKRGRGEVDAVTGKPKRKRRSRKNGTLFPEQVPSGPGFGEAGAEWAGDKGGGWAPHHGHPGGQAGRNCGFQGTEARAFASTGLESGASGRGSYYSTGAPSGQTELSQERQNLFTGYFRSLLDSDDSSDLLDFALSASRPESRKASGTYAGPPTSALPAQRGLATFPSRGAKASPVAVGSSGAGADPSFQPVLSARQTFPPGRAASYGLTPAASDCRAAETFPKLVPPPSAMARSPTTHPPANTYLPQYGGYGAGQSVFAPTKPFTGQDCANSKDCSFAYGSGNSLPASPSSAHSAGYAPPPTGGPCLPPSKASFFSSSEGAPFSGSAPTPLRCDSRASTVSPGGYMVPKGTTASATSAASAASSSSSSFQPSPENCRQFAGASQWPFRQGYGGLDWASEAFSQLYNPSFDCHVSEPNVILDISNYTPQKVKQQTAVSETFSESSSDSTQFNQPVGGGGFRRANSEASSSEGQSSLSSLEKLMMDWNEASSAPGYNWNQSVLFQSSSKPGRGRRKKVDLFEASHLGFPTSASAAASGYPSKRSTGPRQPRGGRGGGACSAKKERGGAAAKAKFIPKPQPVNPLFQDSPDLGLDYYSGDSSMSPLPSQSRAFGVGERDPCDFIGPYSMNPSTPSDGTFGQGFHCDSPSLGAPELDGKHFPPLAHPPTVFDAGLQKAYSPTCSPTLGFKEELRPPPTKLAACEPLKHGLQGASLGHAAAAQAHLSCRDLPLGQPHYDSPSCKGTAYWYPPGSAARSPPYEGKVGTGLLADFLGRTEAACLSAPHLASPPATPKADKEPLEMARPPGPPRGPAAAAAGYGCPLLSDLTLSPVPRDSLLPLQDTAYRYPGFMPQAHPGLGGGPKSGFLGPMAEPHPEDTFTVTSL

SEQ ID NO: 3 - protein, 316 aa, Mus musculus
MATFVELSTKAKMPIVGLGTWKSPPNQVKEAVKAAIDAGYRHIDCAYAYCNENEVGEAIQEKIKEKAVQREDLFIVSKLWPTCFEKKLLKEAFQKTLTDLKLDYLDLYLIHWPQGLQPGKELFPKDDQGRILTSKTTFLEAWEGMEELVDQGLVKALGVSNFNHFQIERLLNKPGLKHKPVTNQVECHPYLTQEKLIQYCHSKGISVTAYSPLGSPDRPSAKPEDPSLLEDPKIKEIAAKHKKTSAQVLIRFHIQRNVVVIPKSVTPSRIQENIQVFDFQLSDEEMATILSFNRNWRACLLPETVNMEEYPYDAEY

SEQ ID NO: 4 - protein, 271 aa, Homo sapiens
MWELRSIAFSRAVFAEFLATLLFVFFGLGSALNWPQALPSVLQIAMAFGLGIGTRVQALGHISGAHINPAVTVACLVGCHVSVLRAAFYVAAQLLGAVAGAALLHEITPADIRGDLAVNALSNSTTAGQAVTVELFLTLQLVLCIFASTDERRGENPGTPALSIGFSVALGHLLGIHYTGCSMNPARSLAPAVVTGKFDDHWVFWIGPLVGAILGSLLYNYVLFPPAKSLSERLAVLKGLEPDTDWEEREVRRRQSVELHSPQSLPRGTKA

SEQ ID NO: 5 - protein, 500 aa, Homo sapiens [sequence omitted]

SEQ ID NO: 6 - protein, 1793 aa, Homo sapiens
MANSTGKAPPDERRKGLAFLDELRQFHHSRGSPFKKIPAVGGKELDLHGLYTRVTTLGGFAKVSEKNQWGEIVEEFNFPRSCSNAAFALKQYYLRYLEKYEKVHHFGEDDDEVPPGNPKPQLPIGAIPSSYNYQQHSVSDYLRQSYGLSMDFNSPNDYNKLVLSLLSGLPNEVDFAINVCTLLSNESKHVMQLEKDPKIITLLLANAGVFDDTLGSFSTVFGEEWKEKTDRDFVKFWKDIVDDNEVRDLISDRNKSHEGTSGEWIWESLFHPPRKLGINDIEGQRVLQIAVILRNLSFEEGNVKLLAANRTCLRFLLLSAHSHFISLRQLGLDTLGNIAAELLLDPVDFKTTHLMFHTVTKCLMSRDRFLKMRGMEILGNLCKAEDNGVLICEYVDQDSYREIICHLTLPDVLLVISTLEVLYMLTEMGDVACTKIAKVEKSIDMLVCLVSMDIQMFGPDALAAVKLIEHPSSSHQMLSEIRPQAIEQVQTQTHVASAPASRAVVAQHVAPPPGIVEIDSEKFACQWLNAHFEVNPDCSVSRAEMYSEYLSTCSKLARGGILTSTGFYKCLRTVFPNHTVKRVEDSSSNGQAHIHVVGVKRRAIPLPIQMYYQQQPVSTSVVRVDSVPDVSPAPSPAGIPHGSQTIGNHFQRTPVANQSSNLTATQMSFPVQGVHTVAQTVSRIPQNPSPHTHQQQNAPVTVIQSKAPIPCEVVKATVIQNSIPQTGVPVSIAVGGGPPQSSVVQNHSTGPQPVTVVNSQTLLHHPSVIPQQSPLHTVVPGQIPSGTPVTVIQQAVPQSHMFGRVQNIPACTSTVSQGQQLITTSPQPVQTSSQQTSAGSQSQDTVIIAPPQYVTTSASNIVSATSVQNFQVATGQMVTIAGVPSPQASRVGFQNIAPKPLPSQQVSSTVVQQPIQQPQQPTQQSVVIVSQPAQQGQTYAPAIHQIVLANPAALPAGQTVQLTGQPNITPSSSPSPVPATNNQVPTAMSSSSTPQSQGPPPTVSQMLSVKRQQQQQHSPAPPPQQVQVQVQQPQQVQMQVQPQQSNAGVGQPASGESSLIKQLLLPKRGPSTPGGKLILPAPQIPPPNNARAPSPQVVYQVASNQAAGFGVQGQTPAQQLLVGQQNVQLVPSAMPPSGGVQTVPISNLQILPGPLISNSPATIFQGTSGNQVTITVVPNTSFAPATVSQGNATQLIAPAGITMSGTQTGVGLPVQTLPATQASPAGQSSCTTATPPFKGDKIICQKEEEAKEATGLHVHERKIEVMENPSCRRGATNTSNGDTKENEMHVGSLLNGRKYSDSSLPPSNSGKIQSETNQCSLISNGPSLELGENGASGKQNSEQIDMQDIKSDLRKPLVNGICDFDKGDGSHLSKNIPNHKTSNHVGNGEISPMEPQGTLDITQQDTAKGDQLERISNGPVLTLGGSSVSSIQEASNAATQQFSGTDLLNGPLASSLNSDVPQQRPSVVVSPHSTTSVIQGHQIIAVPDSGSKVSHSPALSSDVRSTNGTAECKTVKRPAEDTDRETVAGIPNKVGVRIVTISDPNNAGCSATMVAVPAGADPSTVAKVAIESAVQQKQQHPPTYVQNVVPQNTPMPPSPAVQVQGQPNSSQPSPFSGSSQPGDPMRKPGQNFMCLWQSCKKWFQTPSQVFYHAATEHGGKDVYPGQCLWEGCEPFQRQRFSFITHLQDKHCSKDALLAGLKQDEPGQAGSQKSSTKQPTVGGTSSTPRAQKAIVNHPSAALMALRRGSRNLVFRDFTDEKEGPITKHIRLTAALILKNIGKYSECGRRNMNED

SEQ ID NO: 7 - protein, 1835 aa, Homo sapiens
MANSTGKAPPDERRKGLAFLDELRQFHHSRGSPFKKIPAVGGKELDLHGLYTRVTTLGGFAKVSEKNQWGEIVEEFNFPRSCSNAAFALKQYYLRYLEKYEKVHHFGEDDDEVPPGNPKPQLPIGAIPSSYNYQQHSVSDYLRQSYGLSMDFNSPNDYNKLVLSLLSGLPNEVDFAINVCTLLSNESKHVMQLEKDPKIITLLLANAGVFDDTLGSFSTVFGEEWKEKTDRDFVKFWKDIVDDNEVRDLISDRNKSHEGTSGEWIWESLFHPPRKLGINDIEGQRVLQIAVILRNLSFEEGNVKLLAANRTCLRFLLLSAHSHFISLRQLGLDTLGNIAAELLLDPVDFKTTHLMFHTVTKCLMSRDRFLKMRGMEILGNLCKAEDNGVLICEYVDQDSYREIICHLTLPDVLLVISTLEVLYMLTEMGDVACTKIAKVEKSIDMLVCLVSMDIQMFGPDALAAVKLIEHPSSSHQMLSEIRPQAIEQVQTQTHVASAPASRAVVAQHVAPPPGIVEIDSEKFACQWLNAHFEVNPDCSVSRAEMYSEYLSTCSKLARGGILTSTGFYKCLRTVFPNHTVKRVEDSSSNGQAHIHVVGVKRRAIPLPIQMYYQQQPVSTSVVRVDSVPDVSPAPSPAGIPHGSQTIGNHFQRTPVANQSSNLTATQMSFPVQGVHTVAQTVSRIPQNPSPHTHQQQNAPVTVIQSKAPIPCEVVKATVIQNSIPQTGVPVSIAVGGGPPQSSVVQNHSTGPQPVTVVNSQTLLHHPSVIPQQSPLHTVVPGQIPSGTPVTVIQQAVPQSHMFGRVQNIPACTSTVSQGQQLITTSPQPVQTSSQQTSAGSQSQDTVIIAPPQYVTTSASNIVSATSVQNFQVATGQMVTIAGVPSPQASRVGFQNIAPKPLPSQQVSSTVVQQPIQQPQQPTQQSVVIVSQPAQQGQTYAPAIHQIVLANPAALPAGQTVQLTGQPNITPSSSPSPVPATNNQVPTAMSSSSTPQSQGPPPTVSQMLSVKRQQQQQHSPAPPPQQVQVQVQQPQQVQMQVQPQQSNAGVGQPASGESSLIKQLLLPKRGPSTPGGKLILPAPQIPPPNNARAPSPQVVYQVASNQAAGFGVQGQTPAQQLLVGQQNVQLVPSAMPPSGGVQTVPISNLQILPGPLISNSPATIFQGTSGNQVTITVVPNTSFAPATVSQGNATQLIAPAGITMSGTQTGVGLPVQTLPATQASPAGQSSCTTATPPFKGDKIICQKEEEAKEATGLHVHERKIEVMENPSCRRGATNTSNGDTKENEMHVGSLLNGRKYSDSSLPPSNSGKIQSETNQCSLISNGPSLELGENGASGKQNSEQIDMQDIKSDLRKPLVNGICDFDKGDGSHLSKNIPNHKTSNHVGNGEISPMEPQGTLDITQQDTAKGDQLERISNGPVLTLGGSSVSSIQEASNAATQQFSGTDLLNGPLASSLNSDVPQQRPSVVVSPHSTTSVIQGHQIIAVPDSGSKVSHSPALSSDVRSTNGTAECKTVKRPAEDTDRETVAGIPNKVGVRIVTISDPNNAGCSATMVAVPAGADPSTVAKVAIESAVQQKQQHPPTYVQNVVPQNTPMPPSPAVQVQGQPNSSQPSPFSGSSQPGDPMRKPGQNFMCLWQSCKKWFQTPSQVFYHAATEHGGKDVYPGQCLWEGCEPFQRQRFSFITHLQDKHCSKDALLAGLKQDEPGQAGSQKSSTKQPTVGGTSSTPRAQKAIVNHPSAALMALRRGSRNLVFRDFTDEKEGPITKHIRLTAALILKNIGKYSECGRRLLKRHENNLSVLAISNMEASSTLAKCLYELNFTVQSKEQEKDSEMLQ

SEQ ID NO: 8 - protein, 1788 aa, Homo sapiens
MANSTGKAPPDERRKGLAFLDELRQFHHSRGSPFKKIPAVGGKELDLHGLYTRVTTLGGFAKVSEKNQWGEIVEEFNFPRSCSNAAFALKQYYLRYLEKYEKVHHFGEDDDEVPPGNPKPQLPIGAIPSSYNYQQHSVSDYLRQSYGLSMDFNSPNDYNKLVLSLLSGLPNEVDFAINVCTLLSNESKHVMQLEKDPKIITLLLANAGVFDDTLGSFSTVFGEEWKEKTDRDFVKFWKDIVDDNEVRDLISDRNKSHEGTSGEWIWESLFHPPRKLGINDIEGQRVLQIAVILRNLSFEEGNVKLLAANRTCLRFLLLSAHSHFISLRQLGLDTLGNIAAELLLDPVDFKTTHLMFHTVTKCLMSRDRFLKMRGMEILGNLCKAEDNGVLICEYVDQDSYREIICHLTLPDVLLVISTLEVLYMLTEMGDVACTKIAKVEKSIDMLVCLVSMDIQMFGPDALAAVKLIEHPSSSHQMLSEIRPQAIEQVQTQTHVASAPASRAVVAQHVAPPPGIVEIDSEKFACQWLNAHFEVNPDCSVSRAEMYSEYLSTCSKLARGGILTSTGFYKCLRTVFPNHTVKRVEDSSSNGQAHIHVVGVKRRAIPLPIQMYYQQQPVSTSVVRVDSVPDVSPAPSPAGIPHGSQTIGNHFQRTPVANQSSNLTATQMSFPVQGVHTVAQTVSRIPQNPSPHTHQQQNAPVTVIQSKAPIPCEVVKATVIQNSIPQTGVPVSIAVGGGPPQSSVVQNHSTGPQPVTVVNSQTLLHHPSVIPQQSPLHTVVPGQIPSGTPVTVIQQAVPQSHMFGRVQNIPACTSTVSQGQQLITTSPQPVQTSSQQTSAGSQSQDTVIIAPPQYVTTSASNIVSATSVQNFQVATGQMVTIAGVPSPQASRVGFQNIAPKPLPSQQVSSTVVQQPIQQPQQPTQQSVVIVSQPAQQGQTYAPAIHQIVLANPAALPAGQTVQLTGQPNITPSSSPSPVPATNNQVPTAMSSSSTPQSQGPPPTVSQMLSVKRQQQQQHSPAPPPQQVQVQVQQPQQVQMQVQPQQSNAGVGQPASGESSLIKQLLLPKRGPSTPGGKLILPAPQIPPPNNARAPSPQVVYQVASNQAAGFGVQGQTPAQQLLVGQQNVQLVPSAMPPSGGVQTVPISNLQILPGPLISNSPATIFQGTSGNQVTITVVPNTSFAPATVSQGNATQLIAPAGITMSGTQTGVGLPVQTLPATQASPAGQSSCTTATPPFKGDKIICQKEEEAKEATGLHVHERKIEVMENPSCRRGATNTSNGDTKENEMHVGSLLNGRKYSDSSLPPSNSGKIQSETNQCSLISNGPSLELGENGASGKQNSEQIDMQDIKSDLRKPLVNGICDFDKGDGSHLSKNIPNHKTSNHVGNGEISPMEPQGTLDITQQDTAKGDQLERISNGPVLTLGGSSVSSIQEASNAATQQFSGTDLLNGPLASSLNSDVPQQRPSVVVSPHSTTSVIQGHQIIAVPDSGSKVSHSPALSSDVRSTNGTAECKTVKRPAEDTDRETVAGIPNKVGVRIVTISDPNNAGCSATMVAVPAGADPSTVAKVAIESAVQQKQQHPPTYVQNVVPQNTPMPPSPAVQVQGQPNSSQPSPFSGSSQPGDPMRKPGQNFMCLWQSCKKWFQTPSQVFYHAATEHGGKDVYPGQCLWEGCEPFQRQRFSFITHLQDKHCSKDALLAGLKQDEPGQAGSQKSSTKQPTVGGTSSTPRAQKAIVNHPSAALMALRRGSRNLVFRDFTDEKEGPITKHIRLTAALILKNIGKYSECGRR

SEQ ID NO: 9 - protein, 181 aa, Homo sapiens
MMLQHPGQVSASEVSASAIVPCLSPPGSLVFEDFANLTPFVKEELRFAIQNKHLCHRMSSALESVTVSDRPLGVSITKAEVAPEEDERKKRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 10 - protein, 1045 aa, Homo sapiens
MEAKVRPSRRSRAQRDRGRRREAARDARAQSPSSGDEPEPSPGKENAGLRGAPPRGAAPAPRTARPPRRRRRESSSQEEEVIDGFAIASFSTLEALEKDMALKPHERKEKWERRLIKKPRESETCPPAEPSENRRPLEAGSPGQDLEPACDGARKVPLQPSKQMKVTVSKGGDRDSDDDSVLEATSSRDPLSDSSAHAVSGRGYSCDSESGPDDKASVGSEKLFAPGTDKGPALEKSEAKAGPVPKVSGLERSRELSAESFLPTASPAPHAAPCPGPPPGSRANPLVKKEPPAPHRHTPQPPPPQPRGLLPTHVPASLGAFAGHSQAAANGLHGLSRSSSAPLGLGKHVSLSPHGPGPHLSTSHLALRSQAQHQLHAAMFAAPPTLPPPPALPASSLVLPGHPADHELLRQELNTRFLVQSAERPGASLGPGALLRAEFHQHQHTHQHTHQHTHQHQHTFAPFPAGLPPTPPAAPPPFDKYAPKLDSPYFRHSSVSFFPSFPPAIPGLPTLLPHPGPFGSLQGAFQPKVSDPYRAVVKVSTCWEGPWQGRTLVPPGRPRGARDSRSLQKTWVGVAPAPLSASILSQKPGRWCAVHVQIAWQIYRHQQKIKEMQLDPHKLEVGAKLDLFGRPPAPGVFAGFHYPQDLARPLFPSTGAAHPASNPFGPSAHPGSFLPTGPLTDPFSRPSTFGGLGSLSSHAFGGLGSHALAPGGSIFAPKEGSSVHGLPSPHEAWNRLHRAPPSFPAPPPWPKSVDAERVSALTNHDREPDNGKEEQERDLLEKTRLLSRASPATPAGHPVSGLLLRAQSELGRSGAPAEREAEPRVKESRSPAKEEAAKMPARASPPHSKAAPGDVKVKEERGEDEASEPPAGGLHPAPLQLGLGRERLGAPGFAWEPFRGLELPRRAFPAAAPAPGSAALLEPPERPYRDREPHGYSPERLRGELERARAPHLPPAAPALDGALLPSLGALHFPRLSPAALHNGLLARTPPAAAALGAPPPLVTAAGPPTPPGPPRSRTTPLGGLGPGEARDYSPSRNPPEVEAR

SEQ ID NO: 11 - protein, 87 aa, Homo sapiens
MAVLVLRLTVVLGLLVLFLTCYADDKPDKPDDKPDDSGKDPKPDFPKFLSLLGTEIIENAVEFILRSMSRSTGFMEFDDNEGKHSSK

SEQ ID NO: 12 - protein, 255 aa, Homo sapiens
MVLLLVILIPVLVSSAGTSAHYEMLGTCRMVCDPYGGTKAPSTAATPDRGLMQSLPTFIQGPKGEAGRPGKAGPRGPPGEPGPPGPMGPPGEKGEPGRQGLPGPPGAPGLNAAGAISAATYSTVPKIAFYAGLKRQHEGYEVLKFDDVVTNLGNHYDPTTGKFTCSIPGIYFFTYHVLMRGGDGTSMWADLCKNNQVRASAIAQDADQNYDYASNSVVLHLEPGDEVYIKLDGGKAHGGNNNKYSTFSGFIIYAD

SEQ ID NO: 13 - protein, 538 aa, Homo sapiens
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 14 - protein, 357 aa, Homo sapiens
MARENGESSSSWKKQAEDIKKIFEFKETLGTGAFSEVVLAEEKATGKLFAVKCIPKKALKGKESSIENEIAVLRKIKHENIVALEDIYESPNHLYLVMQLVSGGELFDRIVEKGFYTEKDASTLIRQVLDAVYYLHRMGIVHRDLKPENLLYYSQDEESKIMISDFGLSKMEGKGDVMSTACGTPGYVAPEVLAQKPYSKAVDCWSIGVIAYILLCGYPPFYDENDSKLFEQILKAEYEFDSPYWDDISDSAKDFIRNLMEKDPNKRYTCEQAARHPWIAGDTALNKNIHESVSAQIRKNFAKSKWRQAFNATAVVRHMRKLHLGSSLDSSNASVSSSLSLASQKDCAYVAKPESLS

SEQ ID NO: 15 - protein, 385 aa, Homo sapiens
MARENGESSSSWKKQAEDIKKIFEFKETLGTGAFSEVVLAEEKATGKLFAVKCIPKKALKGKESSIENEIAVLRKIKHENIVALEDIYESPNHLYLVMQLVSGGELFDRIVEKGFYTEKDASTLIRQVLDAVYYLHRMGIVHRDLKPENLLYYSQDEESKIMISDFGLSKMEGKGDVMSTACGTPGYVAPEVLAQKPYSKAVDCWSIGVIAYILLCGYPPFYDENDSKLFEQILKAEYEFDSPYWDDISDSAKDFIRNLMEKDPNKRYTCEQAARHPWIAGDTALNKNIHESVSAQIRKNFAKSKWRQAFNATAVVRHMRKLHLGSSLDSSNASVSSSLSLASQKDCLAPSTLCSFISSSSGVSGVGAERRPRPTTVTAVHSGSK

SEQ ID NO: 16 - protein, 354 aa, Mus musculus
MPHRSLRATVVLLLVILKKQPSSSAPLNGSKWTYVGPAGEKNWSKKYPSCGGLLQSPIDLHSDILQYDASLAPLQFQGYNVSVEKLLNLTNDGHSVRLNLNSDMYIQGLQPHHYRAEQLHLHWGNRNDPHGSEHTVSGKHFAAELHIVHYNSDLYPDFSTASDKSEGLAVLAVLIEIGSANPSYDKIFSHLQHVKYKGQQVLIPGFNIEELLPESPGEYYRYEGSLTTPPCYPTVLWTVFRNPVQISQEQLLALETALYFTHMDDPTPREMINNFRQVQKFDERLVYISFRQGLLTDTGLSLGIILSVALAGVLGISIVLAVSIWLFKRKKSKKGDNKGVIYKPAIKKEAEVHA

SEQ ID NO: 17 - protein, 262 aa, Homo sapiens
MVAWRSAFLVCLAFSLATLVQRGSGDFDDFNLEDAVKETSSVKQPWDHTTTTTTNRPGTTRAPAKPPGSGLDLADALDDQDDGRRKPGIGGRERWNHVTTTTKRPVTTRAPANTLGNDFDLADALDDRNDRDDGRRKPIAGGGGFSDKDLEDIVGGGEYKPDKGKGDGRYGSNDDPGSGMVAEPGTIAGVASALAMALIGAVSSYISYQQKKFCFSIQQGLNADYVKGENLEAVVCEEPQVKYSTLHTQSAEPPPPPEPARI

SEQ ID NO: 18 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 19 - protein, 464 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 20 - protein, 461 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTERQNLTMLPRLDSNSWAQAILPSVSQSAEITDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 21 - protein, 430 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 22 - protein, 368 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGSSGPLQ

SEQ ID NO: 23 - protein, 468 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRTTPMTHLTR

SEQ ID NO: 24 - protein, 1899 aa, Homo sapiens
MRSRLPPALAALGAALLLSSIEAEGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPQGPQGESRTGPPGSTGSRGPPGPPGRPGNSGIRGPPGPPGYCDSSQCASIPYNGQSYPGSG

SEQ ID NO: 25 - protein, 1500 aa, Homo sapiens
MTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 26 - protein, 276 aa, Homo sapiens
MDGPAIITQVTNPKEDEGRLPGAGEKASQCNVSLKKQRSRSILSSFFCCFRDYNVEAPPPSSPSVLPPLVEENGGLQKGDQRQVIPIPSPPAKYLLPEVTVLDYGKKCVVIDLDETLVHSSFKPISNADFIVPVEIDGTIHQVYVLKRPHVDEFLQRMGQLFECVLFTASLAKYADPVADLLDRWGVFRARLFRESCVFHRGNYVKDLSRLGRELSKVIIVDNSPASYIFHPENAVPVQSWFDDMTDTELLDLIPFFEGLSREDDVYSMLHRLCNR

SEQ ID NO: 27 - protein, 265 aa, Homo sapiens
MDGPAIITQVTNPKEDEGRLPGAGEKASQCNVSLKKQRSRSILSSFFCCFRDYNVEAPPPSSPSVLPPLVEENGGLQKPPAKYLLPEVTVLDYGKKCVVIDLDETLVHSSFKPISNADFIVPVEIDGTIHQVYVLKRPHVDEFLQRMGQLFECVLFTASLAKYADPVADLLDRWGVFRARLFRESCVFHRGNYVKDLSRLGRELSKVIIVDNSPASYIFHPENAVPVQSWFDDMTDTELLDLIPFFEGLSREDDVYSMLHRLCNR

SEQ ID NO: 28 - protein, 111 aa, Homo sapiens
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 29 - protein, 107 aa, Homo sapiens [sequence omitted]

SEQ ID NO: 30 - protein, 285 aa, Homo sapiens
MAGLGHPAAFGRATHAVVRALPESLGQHALRSAKGEEVDVARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 31 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 32 - protein, 645 aa, Homo sapiens
MSDKSDLKAELERKKQRLAQIREEKKRKEEERKKKEADMQQKKEPVQDDSDLDRKRRETEALLQSIGISPEPPLVQPLHFLTWDTCYFHYLVPTPMSPSSKSVSTPSEAGSQDSGDLGPLTRTLQWDTDPSVLQLQSDSELGRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGELAVPHNDEWTRFARTLVEIRANRADSEEEGTVELSA

SEQ ID NO: 33 - protein, 628 aa, Homo sapiens
MSDKSDLKAELERKKQRLAQIREEKKRKEEERKKKEADMQQKKEPVQDDSDLDRKRRETEALLQSIGISPEPPLVPTPMSPSSKSVSTPSEAGSQDSGDLGPLTRTLQWDTDPSVLQLQSDSELGRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGELAVPHNDEWTRFARTLVEIRANRADSEEEGTVELSA

SEQ ID NO: 34 - protein, 608 aa, Homo sapiens
MSDKSDLKAELERKKQRLAQIREEKKRKEEERKKKEADMQQKKEPVQDDSDLDRKRRETEALLQSIGISPEPPLVPTPMSPSSKSVSTPSEAGSQDSGDLGPLTRRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGELAVPHNDEWTRFARTLVEIRANRADSEEEGTVELSA

SEQ ID NO: 35 - protein, 625 aa, Homo sapiens
MSDKSDLKAELERKKQRLAQIREEKKRKEEERKKKEADMQQKKEPVQDDSDLDRKRRETEALLQSIGISPEPPLVQPLHFLTWDTCYFHYLVPTPMSPSSKSVSTPSEAGSQDSGDLGPLTRRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGELAVPHNDEWTRFARTLVEIRANRADSEEEGTVELSA

SEQ ID NO: 36 - protein, 602 aa, Homo sapiens
MSDKSDLKAELERKKQRLAQIREEKKRKEEERKKKEADMQQKKEPVQDDSDLDRKRRETEALLQSIGISPEPPLVPTPMSPSSKSVSTPSEAGSQDSGDLGPLTRRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGEGLAMLPGWSQNSWTQAILLCWPPKVLGLQT

SEQ ID NO: 37 - protein, 314 aa, Homo sapiens
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD

SEQ ID NO: 38 - protein, 555 aa, Homo sapiens
MTLRAAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGSGPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 39 - protein, 611 aa, Homo sapiens
MSCKKQRSRKHSVNEKCNMKIEHYFSPVSKEQQNNCSTSLMRMESRGDPRATTNTQAQRFHSPKKNPEDQTMPQNRTIYVTLKVNHRRNQDMKLKLTHSENSSLYMALNTLQAVRKEIETHQGQEMLVRGTEGIKEYINLGMPLSCFPEGGQVVITFSQSKSKQKEDNHIFGRQDKASTECVKFYIHAIGIGKCKRRIVKCGKLHKKGRKLCVYAFKGETIKDALCKDGRFLSFLENDDWKLIENNDTILESTQPVDELEGRYFQVEVEKRMVPSAAASQNPESEKRNTCVLREQIVAQYPSLKRESEKIIENFKKKMKVKNGETLFELHRTTFGKVTKNSSSIKVVKLLVRLSDSVGYLFWDSATTGYATCFVFKGLFILTCRHVIDSIVGDGIEPSKWATIIGQCVRVTFGYEELKDKETNYFFVEPWFEIHNEELDYAVLKLKENGQQVPMELYNGITPVPLSGLIHIIGHPYGEKKQIDACAVIPQGQRAKKCQERVQSKKAESPEYVHMYTQRSFQKIVHNPDVITYDTEFFFGASGSPVFDSKGSLVAMHAAGFAYTYQNETRSIIEFGSTMESILLDIKQRHKPWYEEVFVNQQDVEMMSDEDL

SEQ ID NO: 40 - protein, 235 aa, Homo sapiens
MRPLAGGLLKVVFVVFASLCAWYSGYLLAELIPDAPLSSAAYSIRSIGERPVLKAPVPKRQKCDHWTPCPSDTYAYRLLSGGGRSKYAKICFEDNLLMGEQLGNVARGINIAIVNYVTGNVTATRCFDMYEGDNSGPMTKFIQSAAPKSLLFMVTYDDGSTRLNNDAKNAIEALGSKEIRNMKFRSSWVFIAAKGLELPSEIQREKINHSDAKNNRYSGWPAEIQIEGCIPKERS

SEQ ID NO: 41 - protein, 594 aa, Homo sapiens [sequence omitted]

SEQ ID NO: 42 - protein, 633 aa, Homo sapiens
MESGPKMLAPVCLVENNNEQLLVNQQAIQILEKISQPVVVVAIVGLYRTGKSYLMNHLAGQNHGFPLGSTVQSETKGIWMWCVPHPSKPNHTLVLLDTEGLGDVEKGDPKNDSWIFALAVLLCSTFVYNSMSTINHQALEQLHYVTELTELIKAKSSPRPDGVEDSTEFVSFFPDFLWTVRDFTLELKLNGHPITEDEYLENALKLIQGNNPRVQTSNFPRECIRRFFPKRKCFVFDRPTNDKDLLANIEKVSEKQLDPKFQEQTNIFCSYIFTHARTKTLREGITVTGNRLGTLAVTYVEAINSGAVPCLENAVITLAQRENSAAVQRAADYYSQQMAQRVKLPTDTLQELLDMHAACEREAIAIFMEHSFKDENQEFQKKFMETTMNKKGDFLLQNEESSVQYCQAKLNELSKGLMESISAGSFSVPGGHKLYMETKERIEQDYWQVPRKGVKAKEVFQRFLESQMVIEESILQSDKALTDREKAVAVDRAKKEAAEKEQELLKQKLQEQQQQMEAQDKSRKENIAQLKEKLQMEREHLLREQIMMLEHTQKVQNDWLHEGFKKKYEEMNAEISQFKRMIDTTKNDDTPWIARTLDNLADELTAILSAPAKLIGHGVKGVSSLFKKHKLPF

SEQ ID NO: 43 - protein, 569 aa, Homo sapiens
MKKKLVVLGLLAVVLVLVIVGLCLWLPSASKEPDNHVYTRAAVAADAKQCSKIGRDALRDGGSAVDAAIAALLCVGLMNAHSMGIGGGLFLTIYNSTTRKAEVINAREVAPRLAFATMFNSSEQSQKGGLSVAVPGEIRGYELAHQRHGRLPWARLFQPSIQLARQGFPVGKGLAAALENKRTVIEQQPVLCEVFCRDRKVLREGERLTLPQLADTYETLAIEGAQAFYNGSLTAQIVKDIQAAGGIVTAEDLNNYRAELIEHPLNISLGDVVLYMPSAPLSGPVLALILNILKGYNFSRESVESPEQKGLTYHRIVEAFRFAYAKRTLLGDPKFVDVTEVVRNMTSEFFAAQLRAQISDDTTHPISYYKPEFYTPDDGGTAHLSVVAEDGSAVSATSTINLYFGSKVRSPVSGILFNNEMDDFSSPSITNEFGVPPSPANFIQPGKQPLSSMCPTIMVGQDGQVRMVVGAAGGTQITTATALAIIYNLWFGYDVKRAVEEPRLHNQLLPNVTTVERNIDQAVTAALETRHHHTQIASTFIAVVQAIVRTAGGWAAASDSRKGGEPAGY

SEQ ID NO: 44 - protein, 278 aa, Homo sapiens [sequence omitted]

SEQ ID NO: 45 - protein, 222 aa, Homo sapiens
MAARPKLHYPNGRGRMESVRWVLAAAGVEFDEEFLETKEQLYKLQDGNHLLFQQVPMVEIDGMKLVQTRSILHYIADKHNLFGKNLKERTLIDMYVEGTLDLLELLIMHPFLKPDDQQKEVVNMAQKAIIRYFPVFEKILRGHGQSFLVGNQLSLADVILLQTILALEEKIPNILSAFPFLQEYTVKLSNIPTIKRFLEPGSKKKPPPDEIYVRTVYNIFRP

SEQ ID NO: 46 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 47 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

SEQ ID NO: 48 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 49 - protein, 333 aa, Homo sapiens
MTENSDKVPIALVGPDDVEFCSPPAYATLTVKPSSPARLLKVGAVVLISGAVLLLFGAIGAFYFWKGSDSHIYNVHYTMSINGKLQDGSMEIDAGNNLETFKMGSGAEEAIAVNDFQNGITGIRFAGGEKCYIKAQVKARIPEVGAVTKQSISSKLEGKIMPVKYEENSLIWVAVDQPVKDNSFLSSKVLELCGDLPIFWLKPTYPKEIQRERREVVRKIVPTTTKRPHSGPRSNPGAGRLNNETRPSVQEDSQAFNPDNPYHQEGESMTFDPRLDHEGICCIECRRSYTHCQKICEPLGGYYPWPYNYQGCRSACRVIMPCSWWVARILGMV

SEQ ID NO: 50 - protein, 708 aa, Homo sapiens
MKDMPLRIHVLLGLAITTLVQAVDKKVDCPRLCTCEIRPWFTPRSIYMEASTVDCNDLGLLTFPARLPANTQILLLQTNNIAKIEYSTDFPVNLTSLDLSQNNLSSVTNINVKKMPQLLSVYLEENKLTELPEKCLSELSNLQELYINHNLLSTISPGAFIGLHNLLRLHLNSNRLQMINSKWFDALPNLEILMIGENPIIRIKDMNFKPLINLRSLVIAGINLTEIPDNALVGLENLESISFYDNRLIKVPHVALQKVVNLKFLDLNKNPINRIRRGDFSNMLHLKELGINNMPELISIDSLAVDNLPDLRKIEATNNPRLSYIHPNAFFRLPKLESLMLNSNALSALYHGTIESLPNLKEISIHSNPIRCDCVIRWMNMNKTNIRFMEPDSLFCVDPPEFQGQNVRQVHFRDMMEICLPLIAPESFPSNLNVEAGSYVSFHCRATAEPQPEIYWITPSGQKLLPNTLTDKFYVHSEGTLDINGVTPKEGGLYTCIATNLVGADLKSVMIKVDGSFPQDNNGSLNIKIRDIHANSVLVSWKASSKILKSSVKWTAFVKTENSHAAQSARIPSDVKVYNLTHLNPSTEYKICIDIPTIYQKNRKKCVNVTTKGLHPDQKEYEKNNTTTLMACLGGLLGIIGVICLISCLSPEMNCDGGHSYVRNYLQKPTFALGELYPPLINLWEAGKEKSTSLKVKATVIGLPTNMS

SEQ ID NO: 51 - protein, 665 aa, Homo sapiens
MDRPAAAAAAGCEGGGGPNPGPAGGRRPPRAAGGATAGSRQPSVETLDSPTGSHVEWCKQLIAATISSQISGSVTSENVSRDYKALRDGNKLAQMEEAPLFPGESIKAIVKDVMYICPFMGAVSGTLTVTDFKLYFKNVERDPHFILDVPLGVISRVEKIGAQSHGDNSCGIEIVCKDMRNLRLAYKQEEQSKLGIFENLNKHAFPLSNGQALFAFSYKEKFPINGWKVYDPVSEYKRQGLPNESWKISKINSNYEFCDTYPAIIVVPTSVKDDDLSKVAAFRAKGRVPVLSWIHPESQATITRCSQPLVGPNDKRCKEDEKYLQTIMDANAQSHKLIIFDARQNSVADTNKTKGGGYESESAYPNAELVFLEIHNIHVMRESLRKLKEIVYPSIDEARWLSNVDGTHWLEYIRMLLAGAVRIADKIESGKTSVVVHCSDGWDRTAQLTSLAMLMLDSYYRTIKGFETLVEKEWISFGHRFALRVGHGNDNHADADRSPIFLQFVDCVWQMTRQFPSAFEFNELFLITILDHLYSCLFGTFLCNCEQQRFKEDVYTKTISLWSYINSQLDEFSNPFFVNYENHVLYPVASLSHLELWVNYYVRWNPRMRPQMPIHQNLKELLAVRAELQKRVEGLQREVATRAVSSSSERGSSPSHSATSVHTSV

SEQ ID NO: 52 - protein, 162 aa, Homo sapiens
MWKVSALLFVLGSASLWVLAEGASTGQPEDDTETTGLEGGVAMPGAEDDVVTPGTSEDRYKSGLTTLVATSVNSVTGIRIEDLPTSESTVHAQEQSPSATASNVATSHSTEKVDGDTQTTVEKDGLSTVTLVGIIVGVLLAIGFIGGIIVVVMRKMSGRYSP

SEQ ID NO: 53 - protein, 254 aa, Homo sapiens [sequence omitted]

SEQ ID NO: 54 - protein, 299 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVAQQEAQRAQFLVEKAKQEQRQKIVQAEGEAEAAKMLGEALSKNPGYIKLRKIRAAQNISKTIATSQNRIYLTADNLVLNLQDESFTRGSDSLIKGKK

SEQ ID NO: 55 - protein, 533 aa, Homo sapiens
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 56 - protein, 758 aa, Homo sapiens
MGGCTVKPQLLLLALVLHPWNPCLGADSEKPSSIPTDKLLVITVATKESDGFHRFMQSAKYFNYTVKVLGQGEEWRGGDGINSIGGGQKVRLMKEVMEHYADQDDLVVMFTECFDVIFAGGPEEVLKKFQKANHKVVFAADGILWPDKRLADKYPVVHIGKRYLNSGGFIGYAPYVNRIVQQWNLQDNDDDQLFYTKVYIDPLKREAINITLDHKCKIFQTLNGAVDEVVLKFENGKARAKNTFYETLPVAINGNGPTKILLNYFGNYVPNSWTQDNGCTLCEFDTVDLSAVDVHPNVSIGVFIEQPTPFLPRFLDILLTLDYPKEALKLFIHNKEVYHEKDIKVFFDKAKHEIKTIKIVGPEENLSQAEARNMGMDFCRQDEKCDYYFSVDADVVLTNPRTLKILIEQNRKIIAPLVTRHGKLWSNFWGALSPDGYYARSEDYVDIVQGNRVGVWNVPYMANVYLIKGKTLRSEMNERNYFVRDKLDPDMALCRNAREMTLQREKDSPTPETFQMLSPPKGVFMYISNRHEFGRLLSTANYNTSHYNNDLWQIFENPVDWKEKYINRDYSKIFTENIVEQPCPDVFWFPIFSEKACDELVEEMEHYGKWSGGKHHDSRISGGYENVPTDDIHMKQVDLENVWLHFIREFIAPVTLKVFAGYYTKGFALLNFVVKYSPERQRSLRPHHDASTFTINIALNNVGEDFQGGGCKFLRYNCSIESPRKGWSFMHPGRLTHLHEGLPVKNGTRYIAVSFIDP

SEQ ID NO: 57 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 58 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 59 - protein, 781 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 60 - protein, 751 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 61 - protein, 809 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 62 - protein, 749 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEEVTKVTKFIEGGDGHLFEDEEIKRLLQGDTPVRKLQANKKVQGSRRRLREGRSQ

SEQ ID NO: 63 - protein, 721 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEDTPVRKLQANKKVQGSRRRLREGRSQ

SEQ ID NO: 64 - protein, 1216 aa, Homo sapiens
MGHLPTGIHGARRLLPLLWLFVLFKNATAFHVTVQDDNNIVVSLEASDVISPASVYVVKITGESKNYFFEFEEFNSTLPPPVIFKASYHGLYYIITLVVVNGNVVTKPSRSITVLTKPLPVTSVSIYDYKPSPETGVLFEIHYPEKYNVFTRVNISYWEGKDFRTMLYKDFFKGKTVFNHWLPGMCYSNITFQLVSEATFNKSTLVEYSGVSHEPKQHRTAPYPPQNISVRIVNLNKNNWEEQSGNFPEESFMRSQDTIGKEKLFHFTEETPEIPSGNISSGWPDFNSSDYETTSQPYWWDSASAAPESEDEFVSVLPMEYENNSTLSETEKSTSGSFSFFPVQMILTWLPPKPPTAFDGFHIHIEREENFTEYLMVDEEAHEFVAELKEPGKYKLSVTTFSSSGSCETRKSQSAKSLSFYISPSGEWIEELTEKPQHVSVHVLSSTTALMSWTSSQENYNSTIVSVVSLTCQKQKESQRLEKQYCTQVNSSKPIIENLVPGAQYQVVIYLRKGPLIGPPSDPVTFAIVPTGIKDLMLYPLGPTAVVLSWTRPYLGVFRKYVVEMFYFNPATMTSEWTTYYEIAATVSLTASVRIANLLPAWYYNFRVTMVTWGDPELSCCDSSTISFITAPVAPEITSVEYFNSLLYISWTYGDDTTDLSHSRMLHWMVVAEGKKKIKKSVTRNVMTAILSLPPGDIYNLSVTACTERGSNTSMLRLVKLEPAPPKSLFAVNKTQTSVTLLWVEEGVADFFEVFCQQVGSSQKTKLQEPVAVSSHVVTISSLLPATAYNCSVTSFSHDSPSVPTFIAVSTMVTEMNPNVVVISVLAILSTLLIGLLLVTLIILRKKHLQMARECGAGTFVNFASLERDGKLPYNWRRSIFAFLTLLPSCLWTDYLLAFYINPWSKNGLKKRKLTNPVQLDDFDAYIKDMAKDSDYKFSLQFEELKLIGLDIPHFAADLPLNRCKNRYTNILPYDFSRVRLVSMNEEEGADYINANYIPGYNSPQEYIATQGPLPETRNDFWKMVLQQKSQIIVMLTQCNEKRRVKCDHYWPFTEEPIAYGDITVEMISEEEQDDWACRHFRINYADEMQDVMHFNYTAWPDHGVPTANAAESILQFVHMVRQQATKSKGPMIIHCSAGVGRTGTFIALDRLLQHIRDHEFVDILGLVSEMRSYRMSMVQTEEQYIFIHQCVQLMWMKKKQQFCISDVIYENVSKS

SEQ ID NO: 65 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 66 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 67 - protein, 397 aa, Homo sapiens
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEINIRIRVPSIEFNANHPFLFFIRHNKTNTILFYGRLCSP

SEQ ID NO: 68 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKIYSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 69 - protein, 201 aa, Homo sapiens
MFPEQQKEEFVSVWVRDPRIQKEDFWHSYIDYEICIHTNSMCFTMKTSCVRRRYREFVWLRQRLQSNALLVQLPELPSKNLFFNMNNRQHVDQRRQGLEDFLRKVLQNALLLSDSSLHLFLQSHLNSEDIEACVSGQTKYSVEEAIHKFALMNRRFPEEDEEGKKENDIDYDSESSSSGLGHSSDDISSHGCKVNTAPQES

SEQ ID NO: 70 - protein, 89 aa, Homo sapiens
MSSQQQKQPCIPPPQLQQQQVKQPCQPPPQEPCIPKTKEPCHPKVPEPCHPKVPEPCQPKVPEPCHPKVPEPCPSIVTPAPAQQKTKQK

SEQ ID NO: 71 - protein, 398 aa, Homo sapiens [sequence omitted]

SEQ ID NO: 72 - protein, 167 aa, Homo sapiens
MASPRTVTIVALSVALGLFFVFMGTIKLTPRLSKDAYSEMKRAYKSYVRALPLLKKMGINSILLRKSIGALEVACGIVMTLVPGRPKDVANFFLLLLVLAVLFFHQLVGDPLKRYAHALVFGILLTCRLLIARKPEDRSSEKKPLPGNAEEQPSLYEKAPQGKVKVS

SEQ ID NO: 73 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 74 - protein, 811 aa, Homo sapiens
MEAARALRLLLVVCGCLALPPLAEPVCPERCDCQHPQHLLCTNRGLRVVPKTSSLPSPHDVLTYSLGGNFITNITAFDFHRLGQLRRLDLQYNQIRSLHPKTFEKLSRLEELYLGNNLLQALAPGTLAPLRKLRILYANGNEISRLSRGSFEGLESLVKLRLDGNALGALPDAVFAPLGNLLYLHLESNRIRFLGKNAFAQLGKLRFLNLSANELQPSLRHAATFAPLRSLSSLILSANNLQHLGPRIFQHLPRLGLLSLRGNQLTHLAPEAFWGLEALRELRLEGNRLSQLPTALLEPLHSLEALDLSGNELSALHPATFGHLGRLRELSLRNNALSALSGDIFAASPALYRLDLDGNGWTCDCRLRGLKRWMGDWHSQGRLLTVFVQCRHPPALRGKYLDYLDDQQLQNGSCADPSPSASLTADRRRQPLPTAAGEEMTPPAGLAEELPPQPQLQQQGRFLAGVAWDGAARELVGNRSALRLSRRGPGLQQPSPSVAAAAGPAPQSLDLHKKPQRGRPTRADPALAEPTPTASPGSAPSPAGDPWQRATKHRLGTEHQERAAQSDGGAGLPPLVSDPCDFNKFILCNLTVEAVGADSASVRWAVREHRSPRPLGGARFRLLFDRFGQQPKFHRFVYLPESSDSATLRELRGDTPYLVCVEGVLGGRVCPVAPRDHCAGLVTLPEAGSRGGVDYQLLTLALLTVNALLVLLALAAWASRWLRRKLRARRKGGAPVHVRHMYSTRRPLRSMGTGVSADFSGFQSHRPRTTVCALSEADLIEFPCDRFMDSAGGGAGGSLRREDRLLQRFAD

SEQ ID NO: 75 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 76 - protein, 123 aa, Homo sapiens
MRTQSLLLLGALLAVGSQLPAVFGRKKGEKSGGCPPDDGPCLLSVPDQCVEDSQCPLTRKCCYRACFRQCVPRVSVKLGSCPEDQLRCLSPMNHLCHKDSDCSGKKRCCHSACGRDCRDPARG

SEQ ID NO: 77 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 78 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 79 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 80 - protein, 112 aa, Homo sapiens [sequence omitted]

SEQ ID NO: 81 - protein, 555 aa, Homo sapiens; identical to SEQ ID NO: 38

(19) United States
(12) Patent Application Publication
(10) Pub. No.: US 2018/0028608 A1
(43) Pub. Date: Feb. 1, 2018

(54) COMPOSITIONS AND METHODS TO DIAGNOSE DIABETES AND/OR TO TREAT NEGATIVE EFFECTS OF DIABETES

(71) Applicant: Wayne State University, Detroit, MI (US)

(72) Inventor: Fu-Shin X. Yu, Troy, MI (US)

(21) Appl. No.: 15/672,170

(22) Filed: Aug. 8, 2017

Related U.S. Application Data
(62) Division of application No. 15/101,325, filed on Jun. 2, 2016.
(60) Provisional application No. 61/910,831, filed on Dec. 2, 2013.

(51) Int. Cl. A61K 38/18; C12Q 1/68

(52) U.S. Cl. CPC A61K 38/1841; C12Q 1/6883; C12Q 2600/158

(57) ABSTRACT

The present disclosure describes a genome-wide transcriptional analysis of differentially expressed genes in diabetic, non-diabetic, normal and wounded tissue. The analysis allowed development of compositions and methods to diagnose diabetes and/or to treat negative effects of diabetes, including delayed wound healing and reduced vision.

### CROSS REFERENCE TO RELATED APPLICATIONS

This application is a divisional of U.S. patent application Ser. No. 15/101,325, filed on Jun. 2, 2016, which is a national phase application based on International Patent Application No. PCT/US2014/068188 filed Dec. 2, 2014, which claims priority to U.S. Provisional Patent Application Ser. No. 61/910,831 filed Dec. 2, 2013, the entire contents of which are incorporated by reference herein.

### STATEMENT OF GOVERNMENT INTEREST

This invention was made with government support under grant no. EY010869, awarded by the National Institutes of Health. The government has certain rights in the invention.

### FIELD OF THE DISCLOSURE

The present disclosure describes a genome-wide transcriptional analysis of differentially expressed genes in diabetic, non-diabetic, normal and wounded tissue. The analysis allowed development of compositions and methods to diagnose diabetes and/or to treat negative effects of diabetes, including delayed wound healing and reduced vision.

### BACKGROUND OF THE DISCLOSURE

Diabetes affects roughly 170 million people worldwide and by the year 2030, this numbers is expected to double. Diabetics often experience slow or limited wound healing. Slow wound healing allows longer time for the development of infections and can contribute to the formation of bed sores and ulcers. Foot ulcers in diabetics are the most common foot injury leading to leg amputation. Many diabetics also suffer from diabetic neuropathies and ocular complications, often leading to reduced vision and/or blindness.

### SUMMARY OF THE DISCLOSURE

The present disclosure describes a genome-wide transcriptional analysis of differentially expressed genes in diabetic, non-diabetic, normal and wounded tissue. The analysis allowed development of compositions and methods to diagnose diabetes and/or to treat negative effects of diabetes. In particular embodiments, the compositions and methods promote wound healing or re-epithelialization in diabetics. In additional embodiments, the compositions and methods reduce the negative effects of diabetic neuropathies, in particular embodiments by promoting wound healing or re-epithelialization. In additional embodiments, the compositions and methods promote health of the eye and maintenance of eye sight in diabetics. The genome-wide transcriptional analysis also identified novel therapeutic targets.

### BRIEF DESCRIPTION OF THE FIGURES

FIGS. 1A and 1B. Expression profiling of genes differentially expressed in epithelial cells in response to wounding in STZ diabetic and control, non-diabetic (NL) rat corneas. (FIG. 1A) To create an epithelial debridement wound, epithelium were marked by 5 mm trephine and epithelial cells within the mark were removed and collected as homeostatic CECs. The wounds were allowed to heal for 42 h and the epithelial cells migrated into the original wound bed were scraped and referred to as healing CECs. The samples were used for RNA preparation and subjected to Affymetrix GeneChip Rat Genome 230 2.0 Array analysis. Binary comparisons were made as indicated, creating differential expression profiles and significance measures corresponding to the indicated effects, each based on the mean and variance of three independent experiments. (FIG. 1B) Venn diagrams indicating overlap of genes induced due to wounding in NL and diabetic (DM) rats. The cut off values are 2-fold difference for healing versus homeostatic and 1.5 for DM versus NL. Each circle represents the pairwise comparison indicated.

FIGS. 2A-2F. Realtime-PCR verification of the expression patterns of 6 selected genes in healing versus homeostatic CECs in NL rat corneas. The RNA samples were obtained as described in FIG. 1A. The scraped CECs from NL and STZ DM rat corneas for creating a wound were marked as 0 h and from the wound bed 42 hours post-wound (hpw) were subjected to real-time PCR with primers specific for S100A9 (FIG. 2A), CXCL14 (FIG. 2B), Wnt2 (FIG. 2C), Ephx2 (FIG. 2D), Vim (FIG. 2E), and MMP12 (FIG. 2F). The data were presented as fold changes over NL, homeostatic CECs (1). Each condition had 3 samples collected from 3 rats, two independent experiments were performed; *p<0.05; ** p<0.01.

FIGS. 3A-3D. mRNA Expression of Transforming Growth Factor beta (TGFβ) isoforms detected by RT-PCR and verification of TGFβ3 expression by realtime-PCR. The RNA samples were obtained as described in FIG. 1A. The scraped CECs from NL and STZ DM rat and mouse corneas for creating a wound were marked as unwounded (UW) and from the wound bed 42 hpw (rats) or 24 hpw (mice) were subjected to RT-PCR with GAPDH as the internal control and IL-1β as a positive control. (FIG. 3A). The data were presented as fold changes over NL, homeostatic CECs (1). Each condition had 3 samples collected from 3 rats/mice, four independent experiments were performed, 2 with Sprague-Dawley (SD) and STZ-SD (FIG. 3B), 1 with Wistar and Goto-Kakizaki (GK) rats (FIG. 3C), and 1 with B6 and STZ-B6 mouse (FIG. 3D); *p<0.05; ** p<0.01.

FIG. 4. Immunohistochemistry of TGFβ3 distribution in healing and UW corneas. The corneas of SD and STZ-SD were wounded and OCT snap frozen at 42 hpw followed by sectioning and immunostaining with antibodies against TGFβ3; DAPI was used to stain nuclei. The low magnification (5×) images entire cornea were taken and image stitched to present the whole from limbus to wound center. Inserts are high magnification (20×) image of the leading edge. The figure is representatives of 3 corneas per condition from 2 independent experiments.

FIGS. 5A-5C. Effects of siRNA knockdown of TGFβ3 on corneal epithelial wound healing in Wistar rats. (FIG. 5A) TGFβ3 specific and control, nonspecific siRNA were injected into subconjunctival space at two sites (10 μl of 10 μM each site) of NL SD rats 6 hours prior to wounding. A 5-mm wound was made in the central cornea and allowed to healing for 42 h, fluorescence stained, and photographed. (FIG. 5B). Changes in the mean of the remaining wound areas in pixels were calculated by Adobe Photoshop software, **P<0.01, n=5. (FIG. 5C). CECs collected during wounding (first 2 lanes) and 42 hpw were extracted and subjected to Western blotting with listed antibodies, two samples out of 5 from each condition were shown with actin as the internal control. These figures are representatives of 5 corneas per condition from 2 independent experiments; ** p<0.01.

FIGS. 6A-6C. Effects of TGFβ3 on epithelial wound closure in GK rat corneas. (FIG. 6A) A total of 40 ng recombinant TGFβ3, with PBS as the control, was injected into subconjunctival space at two sites (10 μl each) of GK rats 4 hours prior to wounding. Corneal epithelial wound of 5-mm diameter was made and allowed to heal for 42 h and then fluorescence stained, and photographed. (FIG. 6B) Changes in the mean of the remaining wound areas in pixels were calculated by Adobe Photoshop software with p value listed, n=5. (FIG. 6C). CECs collected during wounding (Con) and 42 hpw in PBS and TGFβ3 treated corneas were extracted and subjected to Western blotting with listed antibodies; two samples out of 5 from post wounding corneas were shown with actin as the internal control. The anti-TGFβ1-3 antibodies recognized both latent (44, 25, 47 kDa, respectively) and active (15, 12, 15 kDa, respectively) forms, giving two bands in Western blotting. These figures are representatives of 5 corneas per condition from 2 independent experiments; ** p<0.01.

FIGS. 7A-7C. Effects of TGFβ3 on epithelial wound closure in B6 and STZ B6 mouse corneas. (FIG. 7A) A total of 14 ng recombinant TGFβ3, with PBS as the control, was injected into subconjunctival space at inferior side of B6 and STZ B6 mice 4 hours prior to wounding. A 2 mm epithelial wound was made and allowed to heal for 16 h in B6 mice and 24 h in STZ B6 mice, and then fluorescence stained, and photographed. 1) NL cornea injected with PBS, 2) NL corneal injected with TGFβ3, 3) DM (STZ) cornea injected with PBS, 4) DM corneal injected with TGFβ3. (FIG. 7B) Changes in the mean of the remaining wound areas in pixels were calculated by Adobe Photoshop software with p value listed, n=5. (FIG. 7C). CECs collected during wounding (first 2 lanes) and 16 hpw (1, 2) or 24 hpw (3, 4) in PBS (1, 3) and TGFβ3 (2, 4) treated corneas were extracted and subjected to realtime-PCR analysis for Serpine 1 expression, N=3, **p<0.01.

FIGS. 8A-8D. Expression and distribution of TGFβ3 in cultured human NL and DM corneas with or without wounding. Human DM corneas (FIGS. 8B and 8D) and age matched controls (FIGS. 8A and 8C) were either directly processed for (FIGS. 8A and 8B) or wounded (6 mm) prior to human cornea organ culture. The wounds were allowed to heal in organ culture setting. After ten days in culture, the corneas were frozen in OCT and Cryostat sectioned. The cryostat sections were stained with rabbit anti-human TGFβ3 antibody with nonspecific rabbit IgG as negative control (Insert c′ in NL healed cornea, FIG. 8C). Photos show merged images of confocal microscopy and are the representative of two subjects in each group.

FIG. 9. Sequences of TGFβ3 isoforms.

FIG. 10. Sequence of sEH.

FIG. 11. Exemplary protein sequences associated with expression of noted gene sequences (by symbol).

### DETAILED DESCRIPTION

Diabetes affects roughly 170 million people worldwide including 20.8 million in the United States. By the year 2030, these numbers are expected to double. Diabetes is a chronic disease that occurs when the pancreas does not produce enough insulin, or when the body cannot effectively use the insulin it produces. Hyperglycemia, or raised blood sugar, is a common effect of uncontrolled diabetes and over time leads to serious damage to many of the body's systems. The presence of diabetes can be confirmed by a random venous plasma glucose concentration of ≧11.1 mmol/l; a fasting plasma glucose concentration ≧7.0 mmol/l (whole blood ≧6.1 mmol/l) and/or a two hour plasma glucose concentration ≧11.1 mmol/l two hours after 75 g anhydrous glucose in an oral glucose tolerance test (OGTT).

Diabetics experience a number of pathological conditions that could be alleviated with early detection of the disease. For example, diabetics often experience slow or limited wound healing. Slow or limited wound healing can lead to development of bed sores, pressure wounds, ulcers, neuropathies and peripheral ischemia. These problems are especially prevalent in hospitals and nursing homes. Another area where diabetics suffer from slow or limited wound healing is in the formation of foot ulcers. Foot ulcers in diabetics are the most common foot injury leading to leg amputation, and complications regarding the wounds of the feet are the most frequent reason for hospitalization of diabetics.

Many diabetics also suffer from diabetic (DM) neuropathy. Depending on the affected nerves, symptoms of DM neuropathy can range from pain and numbness in extremities to problems with the digestive system, urinary tract, blood vessels and heart. For some diabetics the systems of DM neuropathy are mild. For others, however, the symptoms can be painful, disabling, and in some instances, fatal.

Diabetes also leads to ocular complications and is the leading cause of blindness around the world. In addition to abnormalities of the retina (retinopathy) and the lens (cataracts), various types of corneal disorders are also common in diabetics. Hyperglycemia significantly alters epithelial structure and function, resulting in basal cell degeneration, decreased cell proliferation, superficial punctate keratitis, breakdown of barrier function, fragility, recurrent erosions and persistent epithelial defects. The corneal defects, termed keratopathy or neurotrophic keratopathy, are likely the results of these pathological changes and are resistant to conventional treatment regimens.

The present disclosure describes a genome-wide transcriptional analysis of differentially expressed genes in diabetic (DM), non-diabetic (NL), normal/unwounded (UW) and wounded/healing (WH) tissue. The analysis allowed development of compositions and methods to diagnose diabetes and/or to treat negative effects of diabetes. In particular embodiments, the compositions and methods promote wound healing or re-epithelialization in diabetics. In additional embodiments, the compositions and methods reduce the negative effects of DM neuropathies. In additional embodiments, the compositions and methods promote health of the eye and maintenance of vision in diabetics.

Diagnosis of Diabetes

The described genome-wide transcriptional analysis of differentially expressed genes in diabetic (DM), non-diabetic (NL), normal/unwounded (UW) and wounded/healing (WH) tissue allowed creation of a panel of biomarkers allowing diagnosis of diabetes. In particular embodiments, an up-regulation of one of the following biomarkers in an UW sample is indicative of diabetes when compared to an UW NL sample.

TABLE 1
Top 10 Up-Regulated Genes in UW DM vs. UW NL Samples:

| Gene | | | Fold- |
|---|---|---|---|
| Symbol | Gene Title | p-value | Change |
| Ephx2* | epoxide hydrolase 2, cytoplasmic | 4.45E−08 | 14.8351 |
| Akr1b8 | aldo-keto reductase family 1, | 0.033642 | 3.35444 |
| | member B8 | | |
| Elovl4 | Elongation of very long chain fatty | 0.030089 | 3.18504 |
| | acids-like 4 | | |
| Ctdspl | CTD (carboxy-terminal domain) | 0.013859 | 2.89265 |
| | small phosphatase-like | | |
| Pdyn* | prodynorphin | 0.024604 | 2.88355 |
| Col12a1 | collagen, type XII, alpha 1 | 0.047762 | 2.69369 |
| CAMK1D | similar to calcium/calmodulin- | 0.000102 | 2.66033 |
| | dependent protein kinase 1D | | |
| Lect1 | leukocyte cell derived chemotaxin 1 | 0.000163 | 2.50341 |
| Phb2 | Prohibitin 2 | 0.014843 | 2.42591 |
| Ddah1 | dimethylarginine | 0.004703 | 2.34997 |
| | dimethylaminohydrolase 1 | | |

*the expression pattern of the genes was confirmed at least by realtime-PCR in 1 or more animal DM models.

In another embodiment, an up-regulation of one or more of the following biomarkers in a WH sample is indicative of diabetes when compared to a WH NL sample.

TABLE 2
Top 10 Up-Regulated Genes in WH DM vs. WH NL Samples:

| Gene | | | Fold- |
|---|---|---|---|
| Symbol | Gene Title | p-value | Change |
| Ephx2* | epoxide hydrolase 2, cytoplasmic | 8.65E−08 | 11.9119 |
| CD99l2 | Similar to MIC2 like 1 | 0.001322 | 3.57749 |
| Arid2 | AT rich interactive domain 2 | 0.007696 | 3.03913 |
| | (Arid-rfx like) | | |
| Ctdspl | CTD (carboxy-terminal domain) | 0.020035 | 2.66501 |
| | small phosphatase-like | | |
| Gsta4 | glutathione S-transferase alpha 4 | 0.005536 | 2.57045 |
| Phgdh | phosphoglycerate dehydrogenase | 0.000145 | 2.50389 |
| Ahdc1 | AT hook, DNA binding motif, | 0.002914 | 2.46515 |
| | containing 1 | | |
| Phb2 | Prohibitin 2 | 0.016485 | 2.37832 |
| CD99L2 | similar to MIC2 like 1 | 0.003917 | 2.26117 |
| Auts2l | autism susceptibility candidate 2-like | 0.007632 | 2.24651 |

*the expression pattern of the genes was confirmed at least by realtime-PCR in 1 or more animal DM models.

In particular embodiments, a down-regulation of one or more of the following markers in a UW sample is indicative of diabetes when compared to a UW NL sample.

TABLE 3
Top 10 Down-regulated Genes in UW DM vs. UW NL Samples:

| Gene | | | Fold- |
|---|---|---|---|
| Symbol | Gene Title | p-value | Change |
| Ggt1 | gamma-glutamyltransferase 1 | 0.023773 | −3.1326 |
| Mtmr1 | Myotubularin related protein 1 | 0.003785 | −3.3812 |
| GBP6 | similar to guanylate binding protein | 0.002009 | −3.3911 |
| | family, member 6 | | |
| Aqp2 | aquaporin 2 (collecting duct) | 0.025883 | −3.777 |
| Krt8 | keratin 8 | 0.000127 | −3.7889 |
| Wfdc5 | WAP four-disulfide core domain 5 | 0.002422 | −4.4483 |
| Gpm6a | glycoprotein m6a | 0.015797 | −4.6711 |
| SNX10 | sorting nexin 10 | 2.27E−05 | −5.5743 |
| Fam111a | family with sequence similarity | 0.037558 | −8.8546 |
| | 111, member A | | |
| C1ql3* | complement component 1, q | 2.65E−06 | −10.238 |
| | subcomponent-like 3 | | |

*the expression pattern of the genes was confirmed at least by realtime-PCR in 1 or more animal DM models.

In further embodiments, a down-regulation of one or more of the following biomarkers in a WH sample is indicative of diabetes when compared to a WH NL sample.

TABLE 4
Top 10 Down-Regulated Genes in WH DM vs. WH NL Samples:

| Gene | | | Fold- |
|---|---|---|---|
| Symbol | Gene Title | p-value | Change |
| Wnt2* | Wingless-type MMTV integration site | 0.019534 | −4.2398 |
| | family member 2 | | |
| Plod2 | procollagen lysine, 2-oxoglutarate | 2.67E−06 | −4.2793 |
| | 5-dioxygenase 2 | | |
| Adm* | adrenomedullin | 0.007971 | −4.5972 |
| Fam3b* | family with sequence similarity 3, | 0.000102 | −5.1147 |
| | member B | | |
| Postn | periostin, osteoblast specific factor | 0.002689 | −5.4259 |
| Ceacam1* | carcinoembryonic antigen-related cell | 0.010465 | −6.4485 |
| | adhesion molecule 1 | | |
| Cald1 | caldesmon 1 | 0.002262 | −6.7921 |
| Tmem35 | transmembrane protein 35 | 0.000227 | −7.5325 |
| Atf3* | activating transcription factor 3 | 0.006267 | −7.5617 |
| Vim* | vimentin | 0.000222 | −8.6761 |

*the expression pattern of the genes was confirmed at least by realtime-PCR in 1 or more animal DM models.

Accordingly, the present disclosure includes methods of assessing samples for up- or down-regulation of one or more of the above-referenced biomarkers to diagnose the presence of diabetes. The biomarkers can be measured in various combinations. One embodiment includes assessing the up-regulation of Ephx2, Akr1b8, ElovI4, Ctdspl and Pdyn. Another embodiment includes assessing the up-regulation of Ephx2, CD99I2, Arid2, Ctdspl and Gsta4. Another embodiment includes assessing the up-regulation of Ephx2, Ctdspl and Pdyn. Another embodiment includes measuring the up-regulation of Ephx2, Akr1b8, ElovI4, CD99I2 and Arid2.

Another embodiment includes assessing the down-regulation of C1qI3, Fam111a, SNX10, Gpm6a, Fibronectin (Fn), Lumican (Lum) and Wfdc5. Another embodiment includes assessing the down-regulation of Vim, Atf3, Tmem35, Cald1 and Ceacam1. Another embodiment includes assessing the down-regulation of C1qI3, Vim, Atf3 and Ceacam1. Another embodiment includes assessing the down-regulation of C1qI3, Fn and Lum.

Additional embodiments include assessing the noted up- and down-regulations in statistically-relevant combinations.

Measurement of Biomarkers

The quantity of one or more biomarkers can be indicated as a value. The value can be one or more numerical values resulting from the evaluation of a sample, and can be derived, e.g., by measuring level(s) of the biomarker(s) in a sample by an assay performed in a laboratory, or from dataset obtained from a provider such as a laboratory, or from a dataset stored on a server. Biomarker levels can be measured using any of several techniques known in the art.

The actual measurement of levels of the biomarkers can be determined at the protein or nucleic acid level using any method known in the art. “Protein” detection includes detection of full-length proteins, mature proteins, pre-proteins, polypeptides, isoforms, mutations, variants, post-translationally modified proteins and variants thereof, and can be detected in any suitable manner. Levels of biomarkers can be determined at the protein level, e.g., by measuring the serum levels of peptides encoded by the gene products described herein, or by measuring the enzymatic activities of these protein biomarkers. Such methods are well-known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the biomarker genes according to the activity of each protein analyzed. For biomarker proteins, polypeptides, isoforms, mutations, and variants thereof known to have enzymatic activity, the activities can be determined in vitro using enzyme assays known in the art. Such assays include, without limitation, protease assays, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the public database entries for the biomarker, expression of the biomarker can be detected and measured using techniques well-known to those of skill in the art. For example, nucleic acid sequences in the sequence databases that correspond to nucleic acids of biomarkers can be used to construct primers and probes for detecting and/or measuring biomarker nucleic acids. These probes can be used in, e.g., Northern or Southern blot hybridization analyses, ribonuclease protection assays, and/or methods that quantitatively amplify specific nucleic acid sequences. As another example, sequences from sequence databases can be used to construct primers for specifically amplifying biomarker sequences in, e.g., amplification-based detection and quantitation methods such as reverse-transcription based polymerase chain reaction (RT-PCR) and PCR. When alterations in gene expression are associated with gene amplification, nucleotide deletions, polymorphisms, post-translational modifications and/or mutations, sequence comparisons in test and reference populations can be made by comparing relative amounts of the examined DNA sequences in the test and reference populations.

As an example, Northern hybridization analysis using probes which specifically recognize one or more of these sequences can be used to determine gene expression. Alternatively, expression can be measured using RT-PCR; e.g., polynucleotide primers specific for the differentially expressed biomarker mRNA sequences reverse-transcribe the mRNA into DNA, which is then amplified in PCR and can be visualized and quantified. Biomarker RNA can also be quantified using, for example, other target amplification methods, such as TMA, SDA, and NASBA, or signal amplification methods (e.g., bDNA), and the like. Ribonuclease protection assays can also be used, using probes that specifically recognize one or more biomarker mRNA sequences, to determine gene expression.

Alternatively, biomarker protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. See WO 04/056456 and WO 04/088309, each of which is hereby incorporated by reference in its entirety. In this regard, other biomarker analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other biomarker metabolites can be similarly detected using reagents that are specifically designed or tailored to detect such metabolites.

In some embodiments, a biomarker is detected by contacting a subject sample with reagents, generating complexes of reagent and analyte, and detecting the complexes. Examples of “reagents” include but are not limited to nucleic acid primers and antibodies.

In some embodiments an antibody binding assay is used to detect a biomarker; e.g., a sample from the subject is contacted with an antibody reagent that binds the biomarker analyte, a reaction product (or complex) including the antibody reagent and analyte is generated, and the presence (or absence) or amount of the complex is determined. The antibody reagent useful in detecting biomarker analytes can be monoclonal, polyclonal, chimeric, recombinant, or a fragment of the foregoing, as discussed in detail above, and the step of detecting the reaction product can be carried out with any suitable immunoassay. The sample from the subject is typically a biological fluid as described above, and can be the same sample of biological fluid as is used to conduct the method described above.

Immunoassays can be homogeneous assays or heterogeneous assays. In a homogeneous assay the immunological reaction can involve the specific antibody (e.g., anti-biomarker protein antibody), a labeled analyte, and the sample of interest. The label produces a signal, and the signal arising from the label becomes modified, directly or indirectly, upon binding of the labeled analyte to the antibody. Both the immunological reaction of binding, and detection of the extent of binding, can be carried out in a homogeneous solution. Immunochemical labels which can be employed include but are not limited to free radicals, radioisotopes, fluorescent dyes, enzymes, bacteriophages, and coenzymes. Immunoassays include competition assays.

In a heterogeneous assay approach, the reagents can be the sample of interest, an antibody, and a reagent for producing a detectable signal. Samples as described above can be used. The antibody can be immobilized on a support, such as a bead (such as protein A and protein G agarose beads), plate or slide, and contacted with the sample suspected of containing the biomarker in liquid phase. The support is separated from the liquid phase, and either the support phase or the liquid phase is examined using methods known in the art for detecting signal. The signal is related to the presence of the analyte in the sample. Methods for producing a detectable signal include but are not limited to the use of radioactive labels, fluorescent labels, or enzyme labels. For example, if the antigen to be detected contains a second binding site, an antibody which binds to that site can be conjugated to a detectable (signal-generating) group and added to the liquid phase reaction solution before the separation step. The presence of the detectable group on the solid support indicates the presence of the biomarker in the test sample. Examples of suitable immunoassays include but are not limited to oligonucleotides, immunoblotting, immunoprecipitation, immunofluorescence methods, chemiluminescence methods, electrochemiluminescence (ECL), and/or enzyme-linked immunoassays (ELISA).

Those skilled in the art will be familiar with numerous specific immunoassay formats and variations thereof which can be useful for carrying out the method disclosed herein. See, e.g., E. Maggio, Enzyme-Immunoassay (1980), CRC Press, Inc., Boca Raton, Fla.; U.S. Pat. No. 4,727,022; U.S. Pat. No. 4,659,678; U.S. Pat. No. 4,376,110; U.S. Pat. No. 4,275,149; U.S. Pat. No. 4,233,402; and, U.S. Pat. No. 4,230,797.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein can likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Antibodies may also be useful for detecting post-translational modifications of biomarkers. Examples of post-translational modifications include, but are not limited to tyrosine phosphorylation, threonine phosphorylation, serine phosphorylation, citrullination and glycosylation (e.g., O-GlcNAc). Such antibodies specifically detect the phosphorylated amino acids in a protein or proteins of interest, and can be used in the immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF). See U. Wirth et al., Proteomics 2002, 2(10):1445-1451.

Kits for Diagnosis

Embodiments disclosed herein include biomarker detection reagents packaged together in the form of a kit for conducting any of the assays described herein. In certain embodiments, the kits include oligonucleotides that specifically identify one or more biomarker nucleic acids based on homology and/or complementarity with biomarker nucleic acids. The oligonucleotide sequences may correspond to fragments of the biomarker nucleic acids. For example, the oligonucleotides can be more than 200, 200, 150, 100, 50, 25, 10, or fewer than 10 nucleotides in length. In other embodiments, the kits include antibodies to proteins encoded by the biomarker nucleic acids. The kits of the present teachings can also include aptamers. The kit can contain in separate containers a nucleic acid or antibody (the antibody either bound to a solid matrix, or packaged separately with reagents for binding to a matrix), control formulations (positive and/or negative), and/or a detectable label, such as but not limited to fluorescein, green fluorescent protein, rhodamine, cyanine dyes, Alexa dyes, luciferase, and radiolabels, among others. Instructions for carrying out the assay, including, optionally, instructions for generating a score, can be included in the kit; e.g., written, tape, VCR, or CD-ROM. The assay can for example be in the form of a Northern hybridization or a sandwich ELISA as known in the art.

While particular gene biomarkers are disclosed, the methods and kits also encompass genetic sequences that hybridize with the specifically disclosed genes. A gene or polynucleotide fragment “hybridizes” to another gene or polynucleotide fragment, such as a cDNA, genomic DNA, or RNA, when a single stranded form of the polynucleotide fragment anneals to the other polynucleotide fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Ed., Cold Spring Harbor Laboratory Press, Cold Spring Harbor (1989), particularly Chapter 11 and Table 11.1 therein (incorporated by reference herein for its teachings regarding the same). The conditions of temperature and ionic strength determine the “stringency” of the hybridization. Stringency conditions can be adjusted to screen for moderately similar fragments (such as homologous sequences from distantly related organisms) to highly similar fragments (such as genes that duplicate functional enzymes from closely related organisms). Post-hybridization washes determine stringency conditions. One set of hybridization conditions to demonstrate that sequences hybridize uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Stringent conditions use higher temperatures in which the washes are identical to those above except for the temperature of the final two 30 min washes in 0.2×SSC, 0.5% SDS is increased to 60° C. Highly stringent conditions use two final washes in 0.1 SSC, 0.1% A SDS at 65° C. Those of ordinary skill in the art will recognize that these temperature and wash solution salt concentrations may be adjusted as necessary according to factors such as the length of the hybridizing sequences.

In some embodiments, biomarker detection reagents can be immobilized on a solid matrix, such as a porous strip, to form at least one biomarker detection site. In some embodiments, the measurement or detection region of the porous strip can include a plurality of sites containing a nucleic acid. In some embodiments, the test strip can also contain sites for negative and/or positive controls. Alternatively, control sites can be located on a separate strip from the test strip. Optionally, the different detection sites can contain different amounts of immobilized nucleic acids, e.g., a higher amount in the first detection site and lesser amounts in subsequent sites. Upon the addition of test sample, the number of sites displaying a detectable signal provides a quantitative indication of the amount of biomarker present in the sample. The detection sites can be configured in any suitably detectable shape and can be, e.g., in the shape of a bar or dot spanning the width of a test strip.

In other embodiments, the kit can contain a nucleic acid substrate array including one or more nucleic acid sequences. The nucleic acids on the array specifically identify one or more nucleic acid sequences represented by one or more of Ephx2, Akr1b8, ElovI4, Ctdspl, Pdyn, CD99I2, Arid2, Gsta4, C1qI3, Fam111a, SNX10, Gpm6, Wfdc5, Vim, Atf3, Tmem35, Cald1 and/or Ceacam1. In various embodiments, the expression of one or more of the sequences represented by one or more of Ephx2, Akr1b8, ElovI4, Ctdspl, Pdyn, CD99I2, Arid2, Gsta4, C1qI3, Fam111a, SNX10, Gpm6, Wfdc5, Vim, Atf3, Tmem35, Cald1 and/or Ceacam1 can be identified by virtue of binding to the array. In some embodiments the substrate array can be on a solid substrate, such as what is known as a “chip.” See, e.g., U.S. Pat. No. 5,744,305. In some embodiments the substrate array can be a solution array; e.g., xMAP (Luminex, Austin, Tex.), Cyvera (Illumina, San Diego, Calif.), RayBio Antibody Arrays (RayBiotech, Inc., Norcross, Ga.), CellCard (Vitra Bioscience, Mountain View, Calif.) and Quantum Dots' Mosaic (Invitrogen, Carlsbad, Calif.).

Promotion of Wound Healing, Reduction of Negative Effects of Neuropathies & Promotion of Eye Health and Vision

Compositions and methods disclosed herein promote wound healing or re-epithelialization, reduce the negative effects of neuropathies and/or promote eye health and maintenance of vision in diabetics by one or more of: up-regulating Transforming Growth Factor (TGF)-β3, up-regulating TGFβ3 phosphorylation, up-regulating Serpine 1 expression, up-regulating fibronectin expression, up-regulating lumican expression down-regulating Ephx2, up-regulating SMAD ⅔ signaling and/or up-regulating PI3K/AKT signaling.

As used herein, “up-regulation” or “up-regulated” means increasing the presence or activity of a protein, increasing the activity of an intracellular signaling pathway and/or increasing the expression of a gene.

The presence or activity of a protein can be up-regulated by one or more of: administering the protein as a therapeutic composition; increasing the expression of the protein; administering or expressing a more active variant of the protein, reducing degradation of the protein following expression, etc. To cause an up-regulation through increased expression of a protein, the copy number of a gene or genes encoding the protein may be increased. Alternatively, a strong and/or inducible promoter may be used to direct the expression of the gene, the gene being expressed either as a transient expression vehicle or homologously or heterologously incorporated into a genome. In another embodiment, the promoter, regulatory region and/or the ribosome binding site upstream of the gene can be altered to achieve over-expression. The expression may also be enhanced by increasing the relative half-life of the messenger or other forms of RNA. Similar mechanisms can be used to up-regulate the expression of genes, for example, Serpine 1, fibronectin or lumican.

The up-regulation of an intracellular signaling pathway or portion thereof can be caused by increasing the presence or activity of a protein within the signaling pathway, increasing the phosphorylation of a protein within the signaling pathway, reducing the degradation of a protein within the pathway or increasing the activity of a protein within the pathway. Up-regulation of an intracellular signaling pathway can also be caused by the down-regulation of a protein.

As used herein, “down-regulation” or “down-regulated” means a decrease in the presence or activity of a protein or gene. A decrease in presence or activity can be caused by: elimination of a protein's activity, translation of an incomplete protein sequence; incorrect folding of protein; reduced transcription of a gene; incomplete transcription of a gene, interference with an encoded RNA transcript, or any other activity resulting in reduced presence, expression or activity of a protein.

A gene may be down-regulated for example by insertion of a foreign set of base pairs in a coding region, deletion of any portion of the gene, or by the presence of antisense sequences that interfere with transcription or translation of the gene. In another embodiment, down-regulation includes elimination of a gene's expression (i.e. gene knockout). In another embodiment, the disruption can occur by optionally inserting a nucleotide or polynucleotide molecule into the native gene sequence whereby the expression of the mutated gene is down-regulated (either partially or completely). In embodiments disclosed herein, Ephx2 is down-regulated.

As is understood by one of ordinary skill in the art, “up-regulation” and “down-regulation” can be measured against a relevant control condition including, without limitation, relative to the activity of a NL subject or sample and/or an UW subject or sample.

TGFβ is a protein that regulates the healing process in humans. In a wound, TGFβ is rapidly released by degranulating platelets and causes a number of effects including: 1) autoinduction of the production of TGFβ by local cells to amplify biological effects; 2) chemoattraction of monocyte/macrophages that debride and sterilize the wound and fibroblasts that begin synthesis of extracellular matrix (ECM); 3) deposition of new ECM by simultaneously stimulating the synthesis of new ECM, inhibiting the proteases that degrade matrix and modulating the numbers of integrin receptors to facilitate cell adhesion to the newly assembled matrix; 4) suppression of the proinflammatory effects of interleukin-1 and tumor necrosis factor; 5) regulation of the action of platelet derived growth factor and fibroblast growth factor so that cell proliferation and angiogenesis are coordinated with matrix deposition; and 6) termination of the process when repair is complete and the wound is closed (Border and Noble, Scientific Amer. Sci. & Med. 2:68-77 (1995)).

Compositions and methods disclosed herein utilize the TGFβ3 isoform of TGFβ to promote wound healing and re-epithelialization in diabetics, reduce the negative effects of DM neuropathies and promote health of the eye and maintenance of eye sight in diabetics.

The serpin peptidase inhibitor, clade E, member 1 (Serpine 1) is located at chromosome 7q22.1. It is expressed as a 402 amino acid protein primarily in liver, smooth muscle cells, adipocytes and platelets; it is also secreted into the plasma. Serpine 1 is an inhibitor of tissue plasminogen activator (tPA) and urokinase (uPA). Examples of compositions that up-regulate Serpine 1 include TGFβ1, TGFβ2, TGFβ3 and epidermal growth factor receptor ligands.

Fibronectin is a glycoprotein present in a soluble dimeric form in plasma, and in a dimeric or multimeric form at the cell surface and in extracellular matrix. Fibronectin is involved in cell adhesion and migration processes including embryogenesis, wound healing, and host defense.

Lumican is a small leucine-rich proteoglycan rich in the cornea and may regulate collagen fibril organization and circumferential growth, corneal transparency, and epithelial cell migration and tissue repair.

Soluble epoxide hydrolase (sEH/Ephx2) hydrolyzes epoxyeicosatrienoic acids, which possess anti-inflammatory, anti-apoptotic, pro-angiogenic, and anti-hypertensive properties, into physiologically less active dihydroxyeicosatrienoic acids. Ephx2 also has anti-oxidative effects, resulting in reduced inflammation and tissue damages

Proteins that share at least 85% identity with TGFβ3, Serpine 1 and sEH are also within the scope of the present disclosure. The % identity can also be at least 86%, at least 87% at least 88%, at least 89%, at least 90%, at least 91%, at least 92%, at least 93%, at least 94%, at least 95%, at least 96%, at least 97%, at least 98% or at least 99%. As is known in the art, “% identity” refers to a relationship between two or more protein sequences, as determined by comparing the sequences. In the art, “identity” also means the degree of sequence relatedness between proteins as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992), each incorporated by reference herein for its teachings regarding the same. Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp (1989) CABIOS. 5:151-153, incorporated by reference herein for its teaching regarding the same) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410, 1990, incorporated by reference herein for its teaching regarding the same); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. incorporated by reference herein for its teaching regarding the same). Within the context of this disclosure it will be understood that where sequence analysis software is used for analysis, the results of the analysis are based on the “default values” of the program referenced. As used herein “default values” will mean any set of values or parameters which originally load with the software when first initialized.

Embodiments disclosed herein include derivatives of the proteins described herein. As used herein, the term “derivatives” refers to proteins having a % identity of at least 85% to TGFβ3, Serpine 1 or sEH or therapeutic proteins disclosed herein as a result of, for example, a sequence substitution, addition, variation, modification, replacement, and/or deletion, of one (or more) amino acid residues. TGFβ3, Serpine 1 or sEH or therapeutic proteins disclosed herein could have a derivative with an Xaa position included in any position, wherein Xaa may be a conservative substitution, deletion, addition, or stop position.

As used herein, a “conservative substitution” involves a substitution of one amino acid for another found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gin); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company which is incorporated by reference for its teachings regarding the same.

In particular embodiments, and regarding TGFβ3 specifically, conservations substitutions can be found in positions 1, 2, 3, 4, 5, 6, 7, 8, 14, 15, 16, 18, 19, 20, 21, 22, 23, 24, 27, 28, 29, 30, 31, 32, 33, 34, 35, 36, 37, 38, 40, 41, 42, 43, 45, 46, 47, 48, 49, 52, 54, 55, 58, 60, 61, 63, 64, 65, 68, 69, 71, 72, 73, 75, 76, 77, 78, 80, 81, 82, 83, 84, 91, 92, 94, 95, 97, 98, 99, 100, 101, 102, 104, 106, 107, 108, 109 or 110.

Homologous sequences are also included within the scope of this disclosure. As is understood by one of ordinary skill in the art, homologous sequences have a common function and evolutionary ancestry, but are found in different species. Any gene or protein sequence not expressly provided herein is readily available and can be found in public databases. Additionally, the DNA sequence of any protein sequence provided can be ascertained using methods commonly known to those of skill in the art.

Prodrugs of TGFβ3 and Serpine 1 or therapeutic proteins disclosed herein can also be used. As used herein, the term “prodrug” refers to a protein that can undergo biotransformation (e.g., either spontaneous or enzymatic) within a subject to release, or to convert (e.g., enzymatically, mechanically, electromagnetically, etc.) an active or more active form of the protein. Prodrugs can be used to overcome issues associated with stability, toxicity, lack of specificity, or limited bioavailability. Exemplary prodrugs include an active protein and a chemical masking group (e.g., a group that reversibly suppresses the activity of the protein). Some preferred prodrugs are variations or derivatives of proteins that have sequences that are cleavable under metabolic conditions. Exemplary prodrugs become active or more active in vivo or in vitro when they undergo a biochemical transformation (e.g., phosphorylation, hydrogenation, dehydrogenation, glycosylation, etc.). Prodrugs often offer advantages of solubility, tissue compatibility, or delayed release (See e.g., Bundgard, Design of Prodrugs, pp. 7-9, 21-24, Elsevier, Amsterdam (1985); and Silverman, The Organic Chemistry of Drug Design and Drag Action, pp. 352-401, Academic Press, San Diego, Calif. (1992) both incorporated by reference for their teachings regarding the same).

As noted, embodiments disclosed herein can also promote wound healing or re-epithelialization, reduce the negative effects of DM neuropathies and/or promote eye health and maintenance of vision in diabetics by up-regulating the intracellular SMAD and/or PI3K/AKT signaling pathways.

SMAD proteins are downstream targets of the transmembrane serine-threonine kinase (STK) receptors that mediate TGFβ signals. SMAD proteins are signal transducers which become phosphorylated by activated type I receptors and then accumulate in the nucleus where they may be involved in transcriptional activation. For example, SMAD2 and SMAD3 are activated by the TGFβ and activin receptors and act in TGFβ/activin signal transduction cascades. SMAD proteins are generally proteins of about 450 amino acids (50-60 kDa) with highly conserved N-terminal and C-terminal domains, linked by a variable, proline-rich, middle region. SMAD proteins have a three-domain structure including the highly conserved C-domain which is necessary for SMAD function in the nucleus. Full-length SMAD polypeptides, for example, SMAD2 and SMAD3, may be activated by phosphorylation near the C-terminus of the polypeptide, which induces a conformational change exposing a binding site in the MH2 domain for transcription factors, such as FAST1, FAST2, FAST3, or Mixer. A SMAD polypeptide in which the N-terminal domain is not present or is truncated may not require phosphorylation in order to expose this binding site. Exemplary compositions that up-regulate SMAD-signaling include TGFβ1, TGFβ2, TGFβ3, activin, and Nodal.

A variety of events can lead to the activation of the PI3K/AKT signaling pathway. For example, signaling from the binding of a growth factor or cytokine, a hematopoietic cytokine, interleukin-3 (IL-3), granulocyte colony-stimulating factor (G-CSF), granulocyte-macrophage colony-stimulating factor (GM-CSF), FMS-like tyrosine kinase-3 (FLT-3), or stem cell factor (SCF) up-regulates the PI3K/AKT signaling pathway. Up-regulated expression of EGFR or cMET also up-regulates the PI3K/AKT signaling pathway as does phosphorylation of HER3 by cMET in the presence of a tyrosine kinase inhibitor.

Compositions that are administered according to the current disclosure can be provided in numerous different forms to achieve up-regulation of TGFβ3, Serpine 1, fibronectin, lumican, TGFβ3 phosphorylation, up-regulating SMAD ⅔ signaling, up-regulating PI3K/AKT signaling, and/or down-regulating of sEH/Ephx2 expression. For example, compositions can be administered in therapeutically and prophylactically effective amounts. As used herein, an “effective amount” is one that achieves promotion of wound healing or re-epithelialization in diabetics, reduction of the negative effects of DM neuropathies in diabetics and/or the promotion of eye health and vision in diabetics. Therapeutically effective amounts promote the healing of an existing condition. Prophylactically effective amounts prevent or reduce the occurrence of a condition or its severity. An effective amount can be assessed according to subjective considerations of a diabetic or treating physician or according to objective measures. Objective measures for the promotion of wound healing include the time required for the closure of an open wound or establishment of a biological barrier. Objective measures for the reduction of negative effects of neuropathies include restoring cornea sensitivity and measurement of sensory nerve density, thickness, and branching.

For skin neuropathy, muscle strength and tone, tendon reflexes, and sensitivity to touch, temperature and vibration can be measured. The measurements can include, without limitation, the Filament test (similar to corneal sensitivity test). Sensitivity to touch may be tested using a soft nylon fiber called a monofilament. If a subject is unable to feel the filament on, for example, the feet, this is a sign of lost sensation in those nerves, Nerve conduction studies, electromyography (EMG), and quantitative sensory testing (noninvasive tests is used to assess how nerves respond to vibration and changes in temperature) can also be used.

Objective measures for the promotion of eye health include epithelial integrity, ocular surface regularity and normal tear secretion. Slit lamp examination of fluorescence staining for epithelial integrity, ocular surface regularity and Schirmer's test uses paper strips inserted into the eye for several minutes to measure the production of tears. Objective measures for the maintenance of vision in humans include no or reduced changes in visual scores (20/20; 20/15; 20/10) as determined by an ophthalmologist.

Corneal sensitivity to touch can be assessed by an aesthesiometer that measures the corneal touch threshold (CTT), which is the reciprocal of corneal sensitivity. Corneal sensitivity can be determined by the corneal touch threshold (CTT) using a Cochet-Bonnet esthesiometer. Five different regions of the cornea can be evaluated (nasal, ventral, lateral, dorsal, and central).

Compositions can also be administered in effective amounts to promote re-epithelialization, reduce the occurrence and/or severity of ulcers and preserve nerve function (appropriate signal potentiation) and/or integrity (physical state of the nerve itself). Objective measures for re-epithelialization include, without limitation, slit lamp microscopy with fluorescence staining. Objective measures for reduction and/or severity of ulcers include, without limitation, slit lamp microscopy for surface (ir)regularity. Objective measures for preservation of nerve function include corneal sensitivity measures. Objective measures for preservation of nerve integrity include in vitro confocal microscopy to determine the nerve fiber health (length, diameters and branches.)

The compositions disclosed herein can additionally be administered with additional components to reduce the occurrence of unwanted events during wound healing or re-epithelialization/neuropathy treatment and or promotion of eye health and maintenance of vision. For example, the compositions can be administered in combination with anti-inflammatory agents including, but not limited to, nonsteroidal anti-inflammatory drugs (NSAIDs), such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids, such as cortisone, hydrocortisone, methylprednisolone, prednisone, prednisolone, betamethasone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

The compositions can also be administered with anti-infective agents including, but not limited to, anthelmintics (mebendazole), antibiotics including aminoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim.

Compositions can also be administered with anesthetics including but not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

Effective amounts as well as dosing regimens can be determined by an attending physician or DM subject, considering various factors such as the age, condition, the severity of the diabetes being treated, time of administration, severity of wound and other clinical factors. Generally, the daily amount or regimen should be in the range of 1 to 10,000 micrograms (μg) of an active ingredient (for example, TGFβ3 or Serpine 1), 1 to 5,000 μg per kilogram, 1 to 1,000 μg or 1 to 100 μg.

In particular embodiments, the compositions can be applied as topical agents (e.g., gels, ointments, pastes, creams, lotions, sprays, powders, salves or ophthalmic formulations including eye drops or injectable formulations), by subcutaneous or subdermal injections and/or as additives to wound dressings.

The gels, ointments, pastes, creams, lotions, sprays, powders, or salves may contain, in addition to compositions of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays particularly may benefit from the inclusion of excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. The compositions of the disclosure can be alternatively administered by aerosol. This is accomplished by preparing an aqueous aerosol, liposomal preparation or solid particles containing a composition of the disclosure. A nonaqueous (e.g., fluorocarbon propellant) suspension could be used. Sonic nebulizers can be preferred because they minimize exposing the compositions to shear, which can result in degradation of the composition.

Ordinarily, an aqueous aerosol is made by formulating an aqueous solution or suspension of the composition together with conventional pharmaceutically-acceptable carriers and stabilizers. The carriers and stabilizers vary with the requirements of the particular composition, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Ophthalmic formulations can be prepared as solutions, suspensions, ointments, gels, emulsions, oils, and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semisolid compositions or sustained release devices or mechanisms that are placed in or around the eye. Aqueous formulations typically can be more than 50%, more than 75%, or more than 90% by weight water.

Ophthalmic formulations can also be provided with tear substitutes. “Tear substitutes” refer to molecules or compositions which lubricate or “wet,” approximate the consistency of endogenous tears, aid in natural tear build-up, or otherwise provide temporary relief of eyes upon ocular administration. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxymethyl cellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion® Tears (Alcon Research, Ltd., Fort Worth, Tex., U.S.A.), Celluvisc® (Allergan, Inc., Irvine, Calif., U.S.A.), Genteal® (Novartis Pharmaceuticals Corporation, East Hanover, N.J., U.S.A.), OccuCoat® (Barnes-Hind, Inc. Clearwater, Fla., U.S.A.), Refresh® (Allergan, Inc., Irvine, Calif., U.S.A.), Systane® (Novartis AG, Basel, Switzerland), Systane Ultra® (Novartis AG, Basel, Switzerland), Refresh Endura® (Allergan, Inc., Irvine, Calif., U.S.A.), Refresh Liquigel® (Allergan, Inc., Irvine, Calif., U.S.A.), Teargen II™ (McKesson), Tears Naturale® (Alcon (Puerto Rico), Inc., Humacao, Puerto Rico), Tears Naturale® II (Alcon (Puerto Rico), Inc., Humacao, Puerto Rico), Tears Naturale Free® (Alcon (Puerto Rico), Inc., Fort Worth, Tex., U.S.A.), and TheraTears® (Advanced Vision Research, Inc., Ann Arbor, Mich., U.S.A.); and polyvinyl alcohols such as Akwa Tears® (Akorn, Inc., Lake Forest, Ill., U.S.A.), HypoTears® (Novartis Pharmaceuticals Corporation, East Hanover, N.J., U.S.A.), Moisture Eyes® (Bausch & Lomb Incorporated, Rochester, N.Y., U.S.A.), Murine Tears® (Medtech Products, Inc., Irvington, N.Y., U.S.A.), and Visine Tears® (Johnson & Johnson, New Brunswick, N.J., U.S.A.), Soothe® (Bausch & Lomb Incorporated, Rochester, N.Y., U.S.A.). Tear substitutes may also include paraffins, such as the commercially available Lacri-Lube® (Allergan, Inc., Irvine, Calif., U.S.A.) ointments. Other commercially available ointments that are used as tear substitutes include Lubrifresh PM™ (Bausch & Lomb Incorporated), Moisture Eyes® PM (Bausch & Lomb Incorporated, Rochester, N.Y., U.S.A.) and Refresh P.M.® (Allergan, Inc., Irvine, Calif., U.S.A.).

Additional potential excipients for ophthalmic formulations include solubilizing agents, stabilizing agents, surfactants, demulcents, viscosity agents, diluents, inert carriers, preservatives, binders, and/or disintegrants. Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Compositions may also be formulated for injection, including subcutaneous, subdermal injection and/or intraocular. Injectable formulations include one or more compositions disclosed herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, or solutes.

Examples of suitable aqueous and nonaqueous carriers, which may be employed in the injectable formulations include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of selected particle size in the case of dispersions, and by the use of surfactants.

Injectable formulations may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a composition, it is desirable to slow the absorption of the composition following injection. This may be accomplished by the use of a liquid suspension of crystalline or amorphous material having poor water solubility. The rate of absorption of the composition then depends upon its rate of dissolution which, in turn, may depend upon crystal size and crystalline form. Alternatively, delayed absorption of a composition can be accomplished by dissolving or suspending the composition in an oil vehicle.

Injectable depot forms are made by forming microencapsule matrices of compositions in biodegradable polymers such as polylactide-polyglycolide. Depending on the ratio of composition to polymer, and the nature of the particular polymer employed, the rate of composition release can be controlled. Examples of other biodegradable polymers include poly(orthoesters) and poly(anhydrides). Depot injectable formulations are also prepared by entrapping the composition in liposomes or microemulsions which are compatible with body tissue.

U.S. Pat. No. 7,918,824 discloses syringes suitable for patient use and is incorporated by reference herein for its teachings regarding the same. The compositions for injection can take such forms as suspensions, solutions or emulsions in oily or aqueous vehicles, and can contain formulatory agents such as suspending, stabilizing, preserving and/or dispersing agents. Alternatively, the compositions can be in powder form and/or lyophilized for constitution with a suitable vehicle, e.g. sterile pyrogen-free water, before use. Lyophilized compositions include less than 5% water content; less than 4.0% water content; or less than 3.5% water content.

Any of the described formulations may also contain pharmaceutically acceptable salts, buffering agents, or preservatives. Examples of such salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES).

For the adjustment of the pH, preferably to a physiological pH, buffers may especially be useful. The pH of the described formulations should be maintained within the range of 4.0 to 8.0, more preferably about 5.5 to 7.5, more preferably about 6.0 to 7.0. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

In certain embodiments, the formulations additionally include a preservative. A preservative may typically be selected from a quaternary ammonium compound such as benzalkonium chloride, benzoxonium chloride or the like. Benzalkonium chloride is better described as: N-benzyl-N—(C8-C18 alkyl)-N,N-dimethylammonium chloride. Further examples of preservatives include antioxidants such as vitamin A, vitamin E, vitamin C, retinyl palmitate, and selenium; the amino acids cysteine and methionine; citric acid and sodium citrate; and synthetic preservatives such as thimerosal, and alkyl parabens, including for example, methyl paraben and propyl paraben. Other preservatives include octadecyldimethylbenzyl ammonium chloride, hexamethonium chloride, benzethonium chloride, phenol, catechol, resorcinol, cyclohexanol, 3-pentanol, m-cresol, phenylmercuric nitrate, phenylmercuric acetate or phenylmercuric borate, sodium perborate, sodium chlorite, alcohols, such as chlorobutanol, butyl or benzyl alcohol or phenyl ethanol, guanidine derivatives, such as chlorohexidine or polyhexamethylene biguanide, sodium perborate, Polyquad®, Germal® II, sorbic acid and stabilized oxychloro complexes (e.g., Purite®). Where appropriate, a sufficient amount of preservative is added to the formulations to ensure protection against secondary contaminations during use caused by bacteria and/or fungi.

Compositions can also be incorporated into transdermal patches and/or wound dressings. Generally, in these embodiments, compositions are embedded within puffs, gauzes, fleeces, gels, powders, sponges or other materials that are associated with a second layer to form an adhesive transdermal patch or wound dressing. Absorption enhancers can also be used to increase the flux of the composition across the skin. The rate of such flux can be controlled by either providing a rate controlling membrane or dispersing the composition in a polymer matrix or gel.

In particular embodiments, the second layer of transdermal patch or wound dressing a can be, without limitation, an elastomeric layer, vapor-permeable film, waterproof film, a woven or nonwoven fabric, mesh, or the like. The composition-containing and second layers can be bonded using any suitable method (e.g., the application of adhesives, such as pressure sensitive adhesives, hot melt adhesives, curable adhesives; the application of heat or pressure, such as in lamination, a physical attachment through the use of stitching, studs, other fasteners; or the like).

Although any adhesive suitable for forming a bond with the skin or other tissue can be used, in certain embodiments a pressure sensitive adhesive is used. Pressure sensitive adhesives are generally defined as adhesives that adhere to a substrate when a light pressure is applied but leave little to no residue when removed. Pressure sensitive adhesives include, but are not limited to, solvent in solution adhesives, hot melt adhesives, aqueous emulsion adhesives, calenderable adhesives, and radiation curable adhesives.

The most commonly used elastomers in pressure sensitive adhesives can include natural rubbers, styrene-butadiene latexes, polyisobutylene, butyl rubbers, acrylics, and silicones. In particular embodiments, acrylic polymer or silicone-based pressure sensitive adhesives can be used. Acrylic polymers can often have a low level of allergenicity, be cleanly removable from skin, possess a low odor, and exhibit low rates of mechanical and chemical irritation. Medical grade silicone pressure sensitive adhesives can be chosen for their biocompatibility.

Amongst the factors that influence the suitability for a pressure sensitive adhesive for use in wound dressings of particular embodiments is the absence of skin irritating components, sufficient cohesive strength such that the adhesive can be cleanly removed from the skin, ability to accommodate skin movement without excessive mechanical skin irritation, and good resistance to body fluids.

In particular embodiments, the pressure sensitive adhesive can include a butyl acrylate. While butyl acrylate pressure sensitive adhesives can generally be used for many applications, any pressure sensitive adhesive suitable for bonding skin can be used. Such pressure sensitive adhesives are well known in the art.

The described formulations can deliver relevant compositions directly or can administer genetic therapies to up- or down-regulate a target. A desired gene can be introduced intracellularly and incorporated within subject cellular DNA for expression, by homologous recombination (Koller and Smithies, 1989, Proc. Natl. Acad. Sci. USA 86:8932-8935; Zijlstra et al., 1989, Nature 342:435-438). In particular embodiments, the desired gene recombinantly expressed in the subject includes an inducible promoter operably linked to the coding region, such that expression of the recombinant gene is controllable by controlling the presence or absence of the appropriate inducer of transcription.

Genetic therapies can be achieved using any method known in the art, including but not limited to transfection, electroporation, microinjection, lipofection, calcium phosphate mediated transfection, infection with a viral or bacteriophage vector containing the gene sequences, cell fusion, chromosome-mediated gene transfer, microcell-mediated gene transfer, sheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present disclosure.

In particular embodiments, and without limitation, retroviral vectors can be used (Miller et al., 1993, Meth. Enzymol. 217:581-599; Boesen et al., 1994, Biotherapy 6:291-302, Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114) adenoviruses can be used (Kozarsky and Wilson, 1993, Current Opinion in Genetics and Development 3:499-503, Rosenfeld et al., 1991, Science 252:431-434; Rosenfeld et al., 1992, Cell 68:143-155; and Mastrangeli et al., 1993, J. Clin. Invest. 91:225-234), Adeno-associated viruses, mammalian artificial chromosomes (Vos, 1998, Curr. Op. Genet. Dev. 8:351-359; liposomes (Tarahovsky and Ivanitsky, 1998, Biochemistry (Mosc) 63:607-618); ribozymes (Branch and Klotman, 1998, Exp. Nephrol. 6:78-83); and triplex DNA can also be used (Chan and Glazer, 1997, J. Mol. Med. 75:267-282). Each of the references cited in this paragraph are incorporated by reference herein for their relevant teachings regarding the same.

### Examples

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of diagnosing diabetes in a subject including comparing a sample obtained from the subject with a control sample wherein altered expression of one or more of Ephx2, Akr1b8, ElovI4, Ctdspl, Pdyn, CD99I2, Arid2, Gsta4, C1qI3, Fam111a, SNX10, Gpm6, Wfdc5, Vim, Atf3, Tmem35, Cald1 and Ceacam1 in the subject sample diagnoses the subject with diabetes.
2. A method of embodiment 1 wherein the altered expression includes up-regulation of one or more of Ephx2, Akr1b8, ElovI4, Ctdspl, Pdyn, CD99I2, Arid2 or Gsta4.
3. A method of embodiment 1 or 2 wherein the altered expression includes up-regulation of one or more of Ephx2, Akr1b8, ElovI4, Ctdspl and Pdyn; Ephx2, CD99I2, Arid2, Ctdspl and Gsta4; Ephx2, Ctdspl and Pdyn; or Ephx2, Akr1b8, ElovI4, CD99I2 and Arid2.
4. A method of any of embodiments 1, 2, or 3 wherein the altered expression includes down-regulation of one or more of C1qI3, Fam111a, SNX10, Gpm6, Wfdc5, Vim, Atf3, Tmem35, Cald1, Fibronectin (Fn), Lumican (Lum) and Ceacam1.
5. A method of any of embodiments 1, 2, 3, 4 or 5 wherein the altered expression includes down-regulation of one or more of C1qI3, Fam111a, SNX10, Gpm6a and Wfdc5; Vim, Atf3, Tmem35, Cald1 and Ceacam1; or C1qI3, Vim, Atf3 and Ceacam1.
6. A method of any of embodiments 1, 2, 3, 4, 5 or 6 further including confirming the diagnosis with a blood glucose test.
7. A method of any of embodiments 1, 2, 3, 4, 5 or 6 including initiating a treatment regimen following the diagnosis.
8. A method of promoting wound healing in a diabetic subject in need thereof including up-regulating TGFβ3 in the subject in need thereof thereby promoting wound healing in the subject.
9. A method of promoting eye health in a diabetic subject in need thereof including up-regulating TGFβ3 in the subject in need thereof thereby promoting eye health in the subject.
10. A method of maintaining vision in a diabetic subject in need thereof including up-regulating TGFβ3 in the subject in need thereof thereby maintaining vision in the subject.
11. A method of promoting re-epithelialization in a diabetic subject in need thereof including up-regulating TGFβ3 in the subject in need thereof thereby promoting re-epithelialization in the subject.
12. A method of reducing the occurrence of ulcers in a diabetic subject in need thereof including up-regulating TGFβ3 in the subject in need thereof thereby reducing the occurrence of ulcers in the subject.
13. A method of preserving nerve function and/or integrity in a diabetic subject in need thereof including up-regulating TGFβ3 in the subject in need thereof thereby preserving nerve function and/or integrity in the subject.
14. A method of any of embodiments 8, 9, 10, 11, 12 or 13 wherein the up-regulating occurs through administering an effective amount of TGFβ3.
15. A method of promoting wound healing in a diabetic subject in need thereof including up-regulating Serpine 1, fibronectin and/or lumican in the subject in need thereof thereby promoting wound healing in the subject.
16. A method of promoting eye health in a diabetic subject in need thereof including up-regulating Serpine 1, fibronectin and/or lumican in the subject in need thereof thereby promoting eye health in the subject.
17. A method of maintaining vision in a diabetic subject in need thereof including up-regulating Serpine 1, fibronectin and/or lumican in the subject in need thereof thereby maintaining vision in the subject.
18. A method of promoting re-epithelialization in a diabetic subject in need thereof including up-regulating Serpine 1, fibronectin and/or lumican in the subject in need thereof thereby promoting re-epithelialization in the subject.
19. A method of reducing the occurrence of ulcers in a diabetic subject in need thereof including up-regulating Serpine 1, fibronectin and/or lumican in the subject in need thereof thereby reducing the occurrence of ulcers in the subject.
20. A method of preserving nerve function and/or integrity in a diabetic subject in need thereof including up-regulating Serpine 1, fibronectin and/or lumican in the subject in need thereof thereby preserving nerve function and/or integrity in the subject.
21. A method of any of embodiments 15, 16, 17, 18, 19 or 20 wherein the up-regulating occurs through administering an effective amount of Serpine 1, TGFβ1, TGFβ2, TGFβ3 or an epidermal growth factor receptor ligand.
22. A method of promoting wound healing in a diabetic subject in need thereof including up-regulating SMAD signaling in the subject in need thereof thereby promoting wound healing in the subject.
23. A method of promoting eye health in a diabetic subject in need thereof including up-regulating SMAD signaling in the subject in need thereof thereby promoting eye health in the subject.
24. A method of maintaining vision in a diabetic subject in need thereof including up-regulating SMAD signaling in the subject in need thereof thereby maintaining vision in the subject.
25. A method of promoting re-epithelialization in a diabetic subject in need thereof including up-regulating SMAD signaling in the subject in need thereof thereby promoting re-epithelialization in the subject.
26. A method of reducing the occurrence of ulcers in a diabetic subject in need thereof including up-regulating SMAD signaling in the subject in need thereof thereby reducing the occurrence of ulcers in the subject.
27. A method of preserving nerve function and/or integrity in a diabetic subject in need thereof including up-regulating SMAD signaling in the subject in need thereof thereby preserving nerve function and/or integrity in the subject.
28. A method of any of embodiments 22, 23, 24, 25, 26 or 27 wherein the SMAD signaling is SMAD2 signaling and/or SMAD3 signaling.
29. A method of any of embodiments 22, 23, 24, 25, 26, 27 or 28 wherein the up-regulating occurs through administering an effective amount of TGFβ1, TGFβ2, TGFβ3, activin or Nodal.
30. A method of promoting wound healing in a diabetic subject in need thereof including up-regulating PI3K/AKT signaling in the subject in need thereof thereby promoting wound healing in the subject.
31. A method of promoting eye health in a diabetic subject in need thereof including up-regulating PI3K/AKT signaling in the subject in need thereof thereby promoting eye health in the subject.
32. A method of maintaining vision in a diabetic subject in need thereof including up-regulating PI3K/AKT signaling in the subject in need thereof thereby maintaining vision in the subject.
33. A method of promoting re-epithelialization in a diabetic subject in need thereof including up-regulating PI3K/AKT signaling in the subject in need thereof thereby promoting re-epithelialization in the subject.
34. A method of reducing the occurrence of ulcers in a diabetic subject in need thereof including up-regulating PI3K/AKT signaling in the subject in need thereof thereby reducing the occurrence of ulcers in the subject.
35. A method of preserving nerve function and/or integrity in a diabetic subject in need thereof including up-regulating PI3K/AKT signaling in the subject in need thereof thereby preserving nerve function and/or integrity in the subject.
36. A method of any of embodiments 30, 31, 32, 33, 34 or 35 wherein the up-regulating occurs through administering an effective amount of a cytokine or growth factor.
37. A method of promoting wound healing in a diabetic subject in need thereof including down-regulating sEH/EPHX2 in the subject in need thereof thereby promoting wound healing in the subject.
38. A method of promoting eye health in a diabetic subject in need thereof including down-regulating sEH/EPHX2 in the subject in need thereof thereby promoting eye health in the subject.
39. A method of maintaining vision in a diabetic subject in need thereof including down-regulating sEH/EPHX2 in the subject in need thereof thereby maintaining vision in the subject.
40. A method of promoting re-epithelialization in a diabetic subject in need thereof including down-regulating sEH/EPHX2 in the subject in need thereof thereby promoting re-epithelialization in the subject.
41. A method of reducing the occurrence of ulcers in a diabetic subject in need thereof including down-regulating sEH/EPHX2 in the subject in need thereof thereby reducing the occurrence of ulcers in the subject.
42. A method of preserving nerve function and/or integrity in a diabetic subject in need thereof including down-regulating sEH/EPHX2 signaling in the subject in need thereof thereby preserving nerve function and/or integrity in the subject.
43. A method of any of embodiments 37, 38, 39, 40, 41 or 42 wherein the down-regulating occurs through administering an effective amount of TGFβ3.

### Example 1

Subjects with diabetes mellitus (DM) often develop corneal complications and delayed wound healing. The aims of this example were to characterize the molecular signatures and biological pathways leading to delayed epithelial wound healing in DM subjects. Genome-wide cDNA microarray analysis revealed 1888 differentially expressed genes in the healing epithelia of normal (NL) versus type 1 DM rat corneas. Gene Ontology and Enrichment analyses indicated TGFβ-signaling as a major altered pathway. Among three TGFβ isoforms, TGFβ1 and β3 were up-regulated in response to wounding in NL corneal epithelial cells (CECs) whereas the latter was greatly suppressed by hyperglycemia in rat type 1 and 2 and mouse type 1 models. Functional analysis indicated that TGFβ3 contributed to wound healing in NL corneas. Moreover, exogenously-added TGFβ3 accelerated epithelial wound closure in type 2 DM rat and type 1 mouse corneas via Smad and PI3K/AKT signaling pathways, auto-regulation, and/or up-regulation of a well-known TGFβ target gene, Serpine 1. Taken together, the Examples demonstrate a comprehensive list of genes differentially expressed in the healing CECs of NL versus DM tissues and suggests the therapeutic potential of one or more of: up-regulating TGFβ3, up-regulating TGFβ3 phosphorylation, up-regulating Serpine 1 expression, down-regulating Ephx2, up-regulating SMAD ⅔ signaling and/or up-regulating PI3K/AKT signaling to promote wound healing, reduce the negative effects of neuropathies and/or promote eye health and maintenance of vision in diabetics.

Methods

Animals and Induction of Diabetes. All investigations conformed to the regulations of the ARVO Statement for the Use of Animals in Ophthalmic and Vision Research, the National Institutes of Health.

Streptozotocin (STZ) induction of type 1 DM in Sprague-Dawley (SD) rats. Seventy male Sprague-Dawley (SD) rats were purchased from Charles River Laboratories (Wilmington, Mass.). These rats (150 g) were divided into two groups. Thirty-five underwent induction of type I DM with an intraperitoneal injection of 55 mg/kg of streptozotocin in ice-cold 0.01 M citrate buffer (pH 4.5), with the controls injected with citrate buffer alone. A second dose of STZ was injected after 4 to 5 days in animals with serum glucose levels less than 200 mg/dL. This regimen produced insulin-deficient DM in 100% of the animals. The animals injected with citrate buffer were normoglycemic. Type 2 Goto-Kakizaki (GK) rats were maintained under standard conditions. C57BL/6 mice were induced to develop type 1 DM according a Low-Dose STZ Induction Protocol (mouse). Particularly, for mice, 50 mg/kg was injected constitutively for 5 days.

Glucose levels and body weight were monitored weekly. Animals with blood sugar levels higher than 400 mg/dL (STZ-SD rats), 220 mg/dL (GK), and 350 mg/dL (STD-mice) were considered diabetic and were used, with age-matched animals as the control, at 8 weeks post STZ treatment for SD rats, 6 months old for GK rats and 10 week post STZ for mice. These are times when epithelial wound closure is significantly delayed and many pathologies can be observed in DM animals.

Corneal Epithelial Debridement Wound. Anesthetized rats and mice were first demarcated with a trephine in the central cornea (5-mm circular wound for rats and 2 mm for mice) and CECs were removed with a blunt scalpel blade under a dissecting microscope (4; 6; 7). The blade with scraped CECs was immediately immersed into liquid nitrogen and the visible ice was collected into a test tube, placed on dry ice and stored at −80° C. The sample was marked as unwounded (UW). Bacitracin ophthalmic ointment was applied to the cornea after surgery to prevent infection. At 42 hours (rats) or 24 hours (mice) post wounding (hours post wounding: “hpw”), the same size trephine was used to mark the original wound, and CECs within the circle were removed, collected as described for the original wounding, and marked as wounded/healing (WH) healing CECs.

RNA Extraction and Real-time PCR. For RNA isolation, CECs were scraped off of corneas, with 2 corneas pooled into 1 tube as one sample. RNA was extracted from the collected CECs using RNeasy Mini Kit (QIAGEN). cDNA was generated with an oligo(dT) primer (Invitrogen) followed by analysis using real-time PCR with the Power SYBR Green PCR Master Mix (AB Applied Biosystems), based on the expression of β-actin.

Gene Array and Functional Analysis. cDNAs were synthesized and hybridized to Affymetrix GeneChip Rat Genome 230. 2.0 array was performed by the Microarray Core Facility at Wayne State University according to the manufacturer's protocols. The microarray data were evaluated by the Genomic Core Lab of National Institute of Diabetes and Digestive and Kidney Diseases. The ANOVA analysis for the robust multi-array (RMA)-normalized data sets was completed by the commercially available microarray data analysis software, Partek Genomic Suite. Signaling pathway and functional network analyses were performed using Genomatrix Pathways System Software.

Subconjunctival Injection of siRNA and Recombinant TGFβ Polypeptides. For mice, 5 μl of solution was injected into the subconjunctival space at 1 site at the superior part of the cornea and for rats, 20 μl at 2 sites, 10 μl each at the superior and inferior quadrants. TGFβ3 was injected 4-6 hours and siRNA 24 hours prior to wounding.

Immunohistochemistry of Rat, Mouse and Human Corneas. Rat eyes were enucleated and embedded in Tissue-Tek OCT compound, and frozen in liquid nitrogen. Six micrometer-thick sections were cut and mounted to polylysine-coated glass slides. Normal and diseased human corneas obtained from the Midwest Eye Bank were embedded in Tissue-Tek OCT compound immediately upon arrival. The cryostat sections of wounded and healed organ-cultured human corneas of DM and age-controlled NL groups were a gift from Dr. Alexander V. Ljubimov, Cedars-Sinai Medical Center (2). After air dry, followed by a 10-min fixation in 4% paraformaldehyde, slides were blocked with 2% BSA in PBS for 1 hour at room temperature (RT). Sections were then incubated with rabbit primary antibody (TGFβ1, TGFβ2 and TGFβ3 1/50, AbCam). This was followed by FITC anti-rabbit antibody (Jackson ImmunoResearch Laboratories 1:100). Slides were mounted with Vectashield mounting medium containing DAPI, and examined under a Carl Zeiss fluorescence microscope Axioplan 2 equipped with an ApoTome digital camera or using confocal microscopy (TCSSP2; Leica). Controls were similarly treated, but the primary antibody was replaced with rat or rabbit IgG.

Determination of Protein Expression and Phosphorylation by Western Blot Analysis. The epithelial cells removed during corneal debridement (the control) and post-wound, but prior to wound closure, were collected from NL and DM rat corneas and lysed with radioimmunoprecipitation assay (RIPA) buffer. Protein concentrations were determined using Micro BCA kits. Levels of various proteins or their phosphorylation were determined using specific antibodies. For each condition, two samples were shown with β-actin levels as the internal controls.

Statistical Analysis. Data were presented as means±standard error. Statistical differences among three or more groups were first identified using one way analysis of variance (ANOVA), followed by Student's t-test for pairwise comparison. Differences were considered statistically significant at p<0.05.

Results

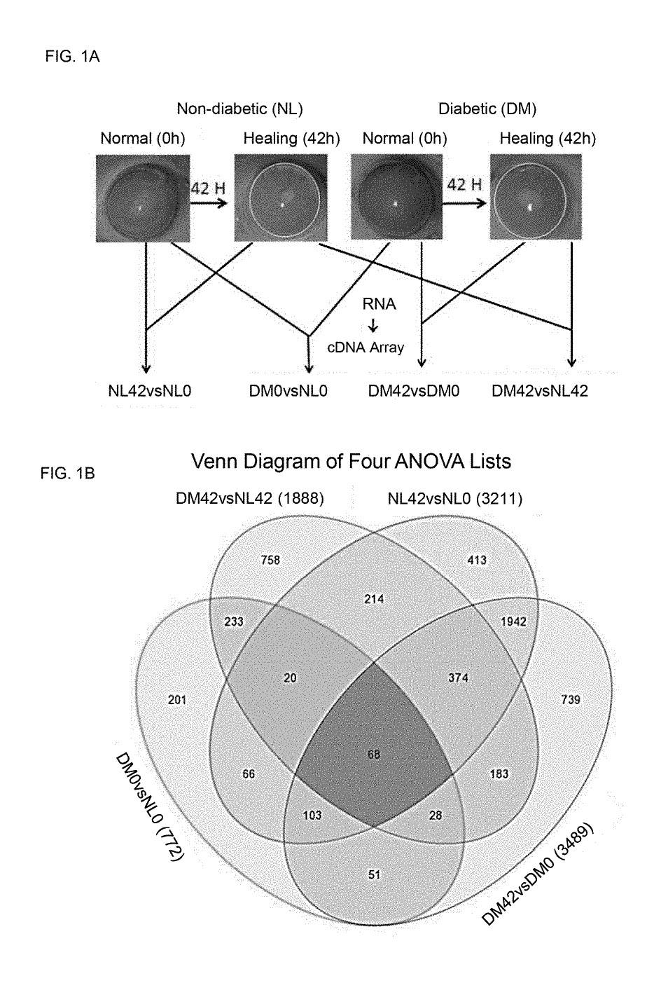

Epithelial wound healing, as seen in DM patients, is significantly delayed in the corneas of STZ-induced type 1 and GK type 2 DM rats compared with NL rats. A genome-wide microarray expression analysis to compare the gene expression profiles of CECs collected during the creation of epithelial debridement wounds (0 hour) and 42 hpw (42 hour) from the NL and from the DM rat corneas (FIG. 1A) was performed. The rats used were 14 weeks old with 8 weeks of hyperglycemia, the duration sufficient to affect the rate of epithelial wound closure. Using Rat Genome GeneChip, 5426 probe sets differentially expressed at least in one of 4 paired comparisons (FIG. 1B) were detected. When healing was compared to UW CECs, a 2.0 fold change cutoff was used, resulting in more than 3000 probe sets differentially expressed in NL and DM corneas. When DM CECs were compared to NL CECs, a 1.5 fold change cutoff was used as a much small number of differentially expressed genes were detected: 772 (397 increase and 377 decrease) in UW CECs and 1888 probe sets (636 increase and 1253 decrease) in WH CECs. Tables 5-9 list the top 10 most up- or down-regulated genes in 4 the paired comparisons:

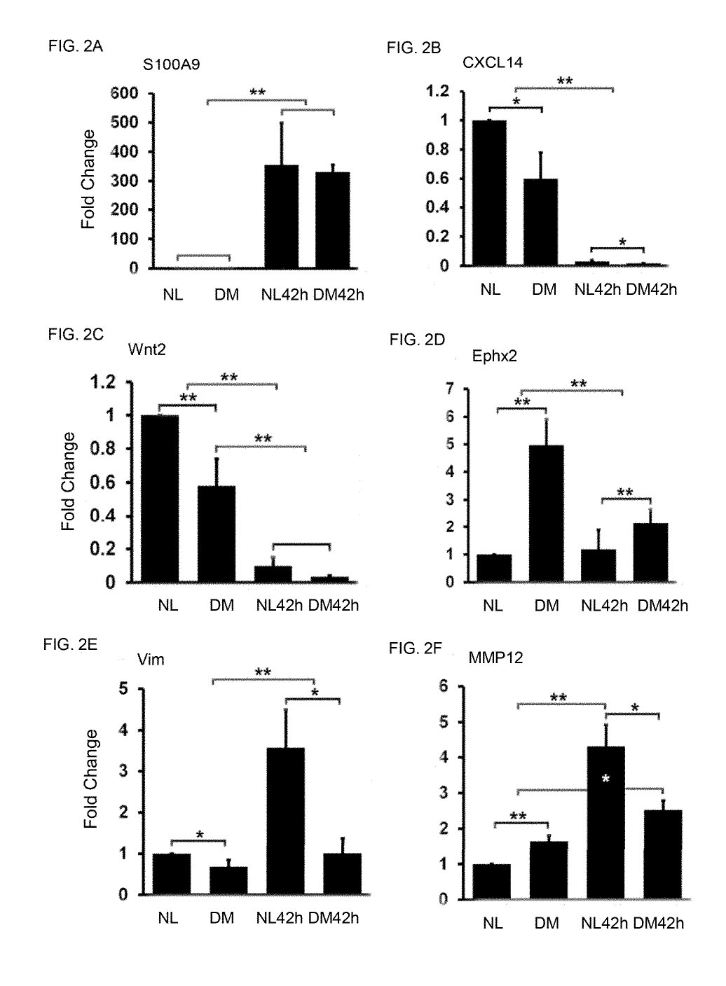

Pair 1: WH versus UW CECs in NL corneas. The most elevated gene is S100A9 (705 fold) which is known to form a heterodimer with S100A8 (249 fold increase), both of which are highly inducible genes in epithelial cells. Its deficiency was found to be associated with non-healing venous leg ulcers. Prominent among greatly down-regulated genes is CXCL14, a homeostatic chemokine that is expressed in basal epidermal keratinocytes. The expression patterns of S100A9 and CXCL14 were verified by realtime-PCR (FIGS. 2A and B).

TABLE 5
Top 10 Up- and Down-regulated
Genes in HW:UW CECs of NL rats

| Gene | | | Fold- |
|---|---|---|---|
| Symbol | Gene Title | p-value | Change |
| S100a9 | S100 calcium binding protein A9 | 1.64E−10 | 705.415 |
| Serpinb2* | serine (or cysteine) peptidase | 2.14E−10 | 563.839 |
| | inhibitor, clade B, member 2 | | |
| Defb4* | defensin beta 4 | 1.03E−09 | 372.452 |
| Car12* | Carbonic anyhydrase 12 | 1.02E−11 | 316.135 |
| Serpinb10* | Serine/cysteine) peptidase inhibitor, | 2.38E−11 | 305.695 |
| | clade B, member 10 | | |
| AVLV472 | similar to AVLV472 | 1.45E−10 | 272.388 |
| Tnc* | Tenascin C | 5.27E−08 | 266.987 |
| S100a8* | S100 calcium binding protein A8 | 9.58E−09 | 249.303 |
| Krt17* | keratin 17 | 2.20E−11 | 202.958 |
| Sprr1b | small proline-rich protein 1B | 1.23E−06 | 202.042 |
| | (cornifin) | | |
| Tril | TLR4 interactor with leucine-rich | 7.17E−07 | −27.792 |
| | repeats | | |
| Dync1i1 | dynein cytoplasmic 1 intermediate | 4.65E−11 | −30.563 |
| | chain 1 | | |
| Igfbp5 | insulin-like growth factor binding | 5.81E−08 | −32.459 |
| | protein 5 | | |
| Lrrn3 | leucine rich repeat neuronal 3 | 1.48E−07 | −34.154 |
| Gpm6a | glycoprotein m6a | 0.000105 | −35.599 |
| Cps1 | Carbamoyl-phosphate synthetase 1 | 9.37E−07 | −41.316 |
| Gad1 | glutamate decarboxylase 1 | 2.44E−08 | −41.548 |
| Tacr2 | tachykinin receptor 2 | 4.29E−07 | −42.053 |
| Cxcl14* | chemokine (C-X-C motif) ligand 14 | 1.54E−08 | −45.971 |
| Ptpro* | protein tyrosine phosphatase, receptor | 1.38E−08 | −56.52 |
| | type, O | | |

*the expression pattern of the genes was confirmed at least by realtime-PCR in 1 or more animal DM models.

Pair 2: WH versus UW CECs in DM corneas. Compared to NL, the WH CECs of DM corneas had 288 more probe sets differentially expressed, and 2497 probe sets showed altered expression in both NL and DM CECs. Among the 10 most highly up-regulated genes, 9 were also found in DM corneas with comparable fold changes. Tenascin C (Tnc) was replaced by podoplanin (Pdpn), a marker of lung injury, in DM CECs. On the down-regulated gene list, Wnt2 was worthy of mention as it was not expressed in UW CEC. The down regulation of Wnt2 in DM, compared to NL WH CECs was verified by realtime-PCR (FIG. 2C).

TABLE 6
Top 10 Up- and Down-regulated
Genes in WH:UW CECs of DM rats

| Gene | | | Fold- |
|---|---|---|---|
| Symbol | Gene Title | p-value | Change |
| S100a9* | S100 calcium binding protein A9 | 2.81E−10 | 458.928 |
| Serpinb10* | serine peptidase inhibitor, clade B, | 2.32E−11 | 311.583 |
| | member 10 | | |
| Car12* | Carbonic anyhydrase 12 | 1.25E−11 | 273.911 |
| Defb4* | defensin beta 4 | 1.85E−09 | 243.856 |
| Krt17* | keratin 17 | 1.99E−11 | 217.086 |
| Sprr1b | small proline-rich protein 1B | 1.24E−06 | 201.148 |
| | (cornifin) | | |
| S100a8* | S100 calcium binding protein A8 | 1.75E−08 | 166.442 |
| AVLV472 | similar to AVLV472 | 3.03E−10 | 165.464 |
| Serpinb2* | serine (or cysteine) peptidase | 1.87E−09 | 124.377 |
| | inhibitor, clade B, member 2 | | |
| Pdpn* | podoplanin | 6.71E−10 | 122.676 |
| Igfbp5 | insulin-like growth factor binding | 1.50E−07 | −21.836 |
| | protein 5 | | |
| Wnt2* | Wingless-type MMTV integration | 0.000251 | −21.992 |
| | site family member 2 | | |
| Dapl1 | death associated protein-like 1 | 1.34E−06 | −23.277 |
| Gpm6a | glycoprotein m6a | 0.000216 | −24.972 |
| Tacr2 | tachykinin receptor 2 | 9.35E−07 | −29.366 |
| Cxcl14* | chemokine (C-X-C motif) ligand 14 | 3.88E−08 | −30.087 |
| Dync1i1 | dynein cytoplasmic 1 intermediate | 4.02E−11 | −32.555 |
| | chain 1 | | |
| Gad1 | glutamate decarboxylase 1 | 3.71E−08 | −34.251 |
| Cps1 | Carbamoyl-phosphate synthetase 1 | 7.29E−07 | −46.747 |
| Ptpro* | protein tyrosine phosphatase, receptor | 1.63E−08 | −51.929 |
| | type, O | | |

*the expression pattern of the genes was confirmed at least by realtime-PCR in 1 or more animal DM models.

Pair 3: DM versus NL UW CECs. Compared to WH versus UW CECs in pairs 1 and 2, there were fewer differentially expressed genes in DM versus NL CECs, with much lower-fold changes. Thus, the cutoff value was lowered to 1.5 fold changes. The most highly up-regulated and down-regulated genes were Ephx2, known to contribute to renal injury during diabetes, and C1q13, a gene found in the liver/biliary and renal systems as well as the central nervous system where it regulates synapse formation, respectively. Up-regulation of Ephx2 in DM CECs was verified by realtime-PCR (FIG. 2D).

TABLE 7
Top 10 Up- and Down-regulated Genes in UW DM:ML rat CECs

| Gene | | | Fold- |
|---|---|---|---|
| Symbol | Gene Title | p-value | Change |
| Ephx2* | epoxide hydrolase 2, cytoplasmic | 4.45E−08 | 14.8351 |
| Akr1b8 | aldo-keto reductase family 1, | 0.033642 | 3.35444 |
| | member B8 | | |
| Elovl4 | Elongation of very long chain fatty | 0.030089 | 3.18504 |
| | acids-like 4 | | |
| Ctdspl | CTD (carboxy-terminal domain) | 0.013859 | 2.89265 |
| | small phosphatase-like | | |
| Pdyn* | prodynorphin | 0.024604 | 2.88355 |
| Col12a1 | collagen, type XII, alpha 1 | 0.047762 | 2.69369 |
| CAMK1D | similar to calcium/calmodulin- | 0.000102 | 2.66033 |
| | dependent protein kinase 1D | | |
| Lect1 | leukocyte cell derived | 0.000163 | 2.50341 |
| | chemotaxin 1 | | |
| Phb2 | Prohibitin 2 | 0.014843 | 2.42591 |
| Ddah1 | dimethylarginine | 0.004703 | 2.34997 |
| | dimethylaminohydrolase 1 | | |
| Ggt1 | gamma-glutamyltransferase 1 | 0.023773 | −3.1326 |
| Mtmr1 | Myotubularin related protein 1 | 0.003785 | −3.3812 |
| GBP6 | similar to guanylate binding | 0.002009 | −3.3911 |
| | protein family, member 6 | | |
| Aqp2 | aquaporin 2 (collecting duct) | 0.025883 | −3.777 |
| Krt8 | keratin 8 | 0.000127 | −3.7889 |
| Wfdc5 | WAP four-disulfide core domain 5 | 0.002422 | −4.4483 |
| Gpm6a | glycoprotein m6a | 0.015797 | −4.6711 |
| SNX10 | sorting nexin 10 | 2.27E−05 | −5.5743 |
| Fam111a | family with sequence similarity | 0.037558 | −8.8546 |
| | 111, member A | | |
| C1ql3* | complement component 1, q | 2.65E−06 | −10.238 |
| | subcomponent-like 3 | | |

*the expression pattern of the genes was confirmed at least by realtime-PCR in 1 or more animal DM models.

Pair 4: DM versus NL WH CECs. Compared to homeostatic cells, a relatively large number of genes were found to be differentially expressed: 1888 probe sets in DM versus NL WH CECs. Among the 105 highly induced probe sets, only 17 probe sets represent known genes, including Ephx2. On the other hand, many probe sets with down-regulated expression in DM, compared to in NL WH CECs, contained gene identities. These include Vimentin, ATF3, Wnt2, Tenascin C, peroxidasin (a gene associated with anterior segment dysgenesis (22)) and MMP12. FIGS. 2E and 2F show the verifications of the expression patterns of Vimentin and MMP12. The expression patterns of 17 genes were confirmed in 1 or more DM models by realtime-PCR and/or immunohistochemistry.

TABLE 8
Top 10 Up- and Down-regulated Genes in WH DM:NL rat CECs

| Gene | | | Fold- |
|---|---|---|---|
| Symbol | Gene Title | p-value | Change |
| Ephx2* | epoxide hydrolase 2, cytoplasmic | 8.65E−08 | 11.9119 |
| CD99l2 | Similar to MIC2 like 1 | 0.001322 | 3.57749 |
| Arid2 | AT rich interactive domain 2 | 0.007696 | 3.03913 |
| | (Arid-rfx like) | | |
| Ctdspl | CTD (carboxy-terminal domain) | 0.020035 | 2.66501 |
| | small phosphatase-like | | |
| Gsta4 | glutathione S-transferase alpha 4 | 0.005536 | 2.57045 |
| Phgdh | phosphoglycerate dehydrogenase | 0.000145 | 2.50389 |
| Ahdc1 | AT hook, DNA binding motif, | 0.002914 | 2.46515 |
| | containing 1 [Rattus | | |
| Phb2 | Prohibitin 2 | 0.016485 | 2.37832 |
| CD99L2 | similar to MIC2 like 1 | 0.003917 | 2.26117 |
| Auts2l | autism susceptibility candidate | 0.007632 | 2.24651 |
| | 2-like | | |
| Wnt2* | Wingless-type MMTV integration | 0.019534 | −4.2398 |
| | site family member 2 | | |
| Plod2 | procollagen lysine, 2-oxoglutarate | 2.67E−06 | −4.2793 |
| | 5-dioxygenase 2 | | |
| Adm* | adrenomedullin | 0.007971 | −4.5972 |
| Fam3b* | family with sequence similarity 3, | 0.000102 | −5.1147 |
| | member B | | |
| Postn | periostin, osteoblast specific factor | 0.002689 | −5.4259 |
| Ceacam1* | carcinoembryonic antigen-related cell | 0.010465 | −6.4485 |
| | adhesion molecule 1 | | |
| Cald1 | caldesmon 1 | 0.002262 | −6.7921 |
| Tmem35 | transmembrane protein 35 | 0.000227 | −7.5325 |
| Atf3* | activating transcription factor 3 | 0.006267 | −7.5617 |
| Vim* | vimentin | 0.000222 | −8.6761 |

*the expression pattern of the genes was confirmed at least by realtime-PCR in 1 or more animal DM models.

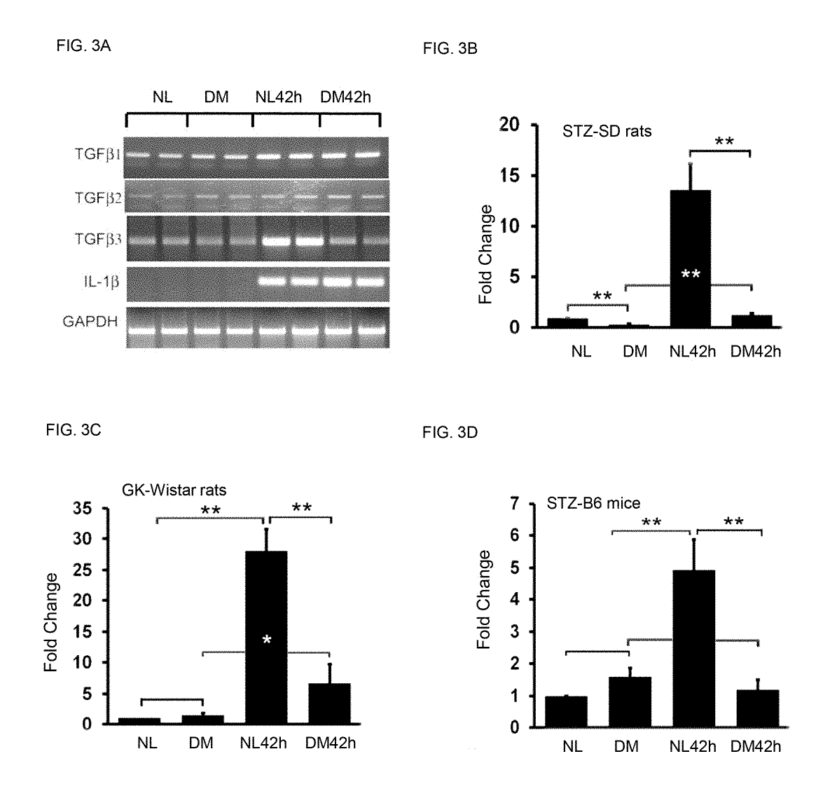

Decreased expression of TGFβ3 in WH CECs of DM corneas. Gene Ontology (GO) analysis of 1888 probe sets revealed many altered signaling pathways including IL-1 (1.02e-3, 22/132, also see FIGS. 3A-3D), HGF (2.8e-314/75), MMP (1.58e-4, 23/124) and TGFβ (7.39e-3, 43/360) mediated pathways. Similar to GO Analysis, enrichment analysis of genes differentially expressed in DM versus NL WH CECs revealed TGFβ-dependent epithelial mesenchymal transition to be a major event altered therein.

Gene Ontology (GO) is a simple, but extremely widely used, systems biology technique for highlighting biological processes. In the analysis, genes are grouped into categories by some common biological property and then tested to find categories that are over represented amongst the differentially expressed genes.

To assess the expression patterns of the TGFβ family, RT PCR was performed (FIG. 3A) using STZ SD rats. All three isoforms were detected while mRNA for IL-1β (as a control) was undetectable in UW NL and DM CECs. Wounding induced IL-1β expression at the mRNA levels and this up-regulation was more apparent in DM WH CECs. The levels of TGFβ1 were also elevated in WH CECs; however, there was no detectable difference between DM and NL corneas (FIG. 36). The levels of TGFβ2 were consistent under 4 conditions. TGFβ3, on the other hand, was greatly up-regulated in the WH CECs of NL, but the levels of TGFβ3 in DM CECs remained at a level comparable to that of UW CECs.

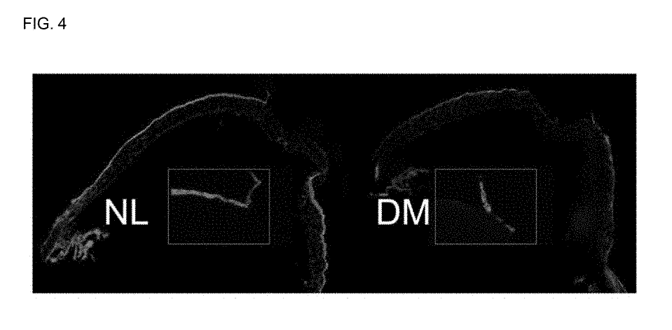

Three rodent DM models were established: STZ SD rats, GK rats, and STZ-m ice. The expression of TGFβ3 was further assessed by real time PCR which showed significant up-regulation in the WH CECs of NL, but not DM, corneas in all 3 rodent models (FIG. 3B-3D). The expression and distribution of TGFβ3 were also assessed using immunohistochemistry (FIG. 4). While TGFβ3 abundantly stained the entire healing corneal epithelial sheet, from the leading edge to the border region between the cornea and limbus in SD rat corneas, there were only a few TGFβ3 positive cells found at the leading edge of the migratory epithelial sheet in type 1 DM rat corneas; the rest of the epithelial sheet stained low or negative.

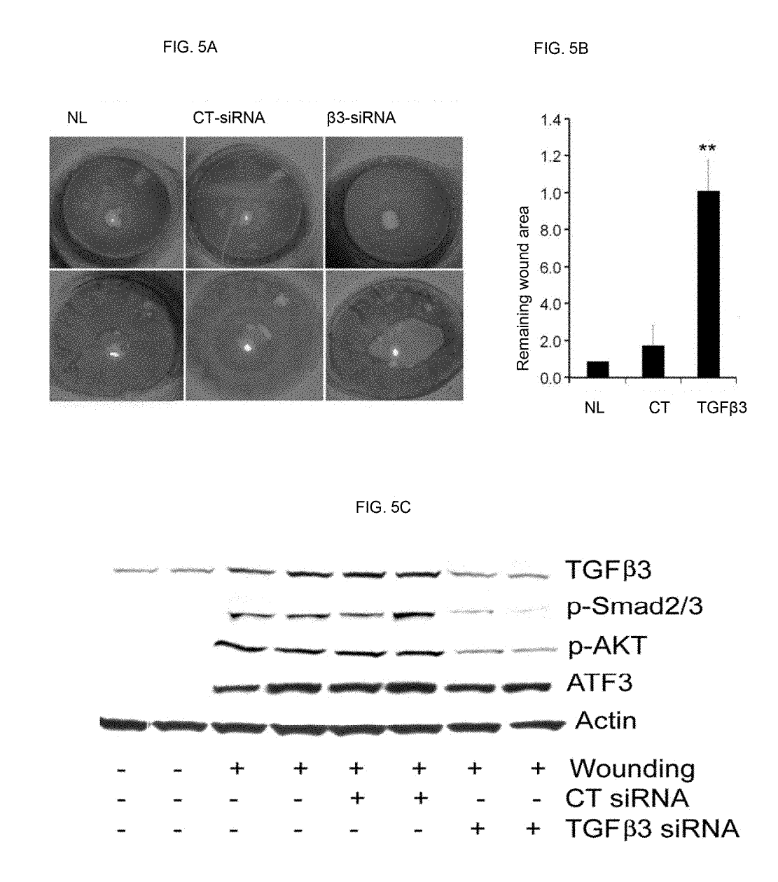

TGFβ 3 is necessary for proper wound healing. Whether TGFβ 3 is required for wound healing in NL rats was next evaluated. Subconjunctival injection of siRNA in Wistar rats was used and a significant delay in TGFβ-specific, but not the control siRNAs was observed as compared to the control rats (FIG. 5A). Western blotting analysis of CECs revealed a basal expression of TGFβ 3 in UW CECs and elevated levels in WH CECs in the Wistar rat and this wounding-induced TGFβ 3 up-regulation was knocked down by TGFβ 3 specific siRNA. In UW CECs, there was no detectable phosphorylation of Smad⅔ and AKT, and in WH CECs, both signaling molecules were phosphorylated. Consistent with down-regulated TGFβ 3 levels, the phosphorylation of these proteins was also dampened, while the expression of ATF3, a stress responsive transcription factor with a similar expression pattern to TGFβ3, was not affected (FIG. 5B).

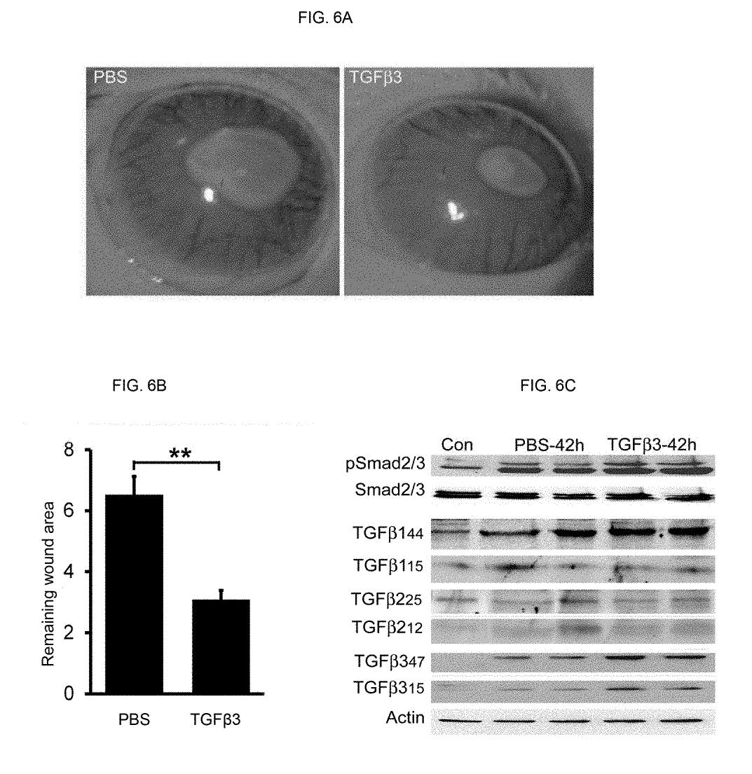

Exogenous TGFβ 3 accelerates delayed epithelial wound closure in DM corneas. Whether exogenously-added TGFβ 3 in DM corneas accelerates delayed epithelial wound closure in GK and STZ B6 mice was assessed next. In GK rat corneas, 40 ng TGFβ 3 in 20 μl was injected 4 hours prior to epithelial debridement; this substantially increasing the rate of wound closure (FIGS. 6A and 6B) and enhancing Smad⅔ phosphorylation in WH CECs (FIG. 6C). To assess if exogenously added TGFβ 3 affects the isoform expression, antibodies against TGFβ 1-3 were used for Western blotting. While TGFβ 1 was up-regulated in WH DM CECs, exogenously-added TGFβ 3 appeared to have no effects on its expression. The levels of TGFβ 2 were low and unchanged in all the samples. Interestingly, exogenously-applied TGFβ 3 increased TGFβ 3 expression. While some elevated active TGFβ 3 molecules could be from exogenously added TGFβ 3 (15 kDa), the latent form (47 kDa) can only be from endogenous sources, indicating an auto-regulation of TGFβ 3 expression in rat corneas.

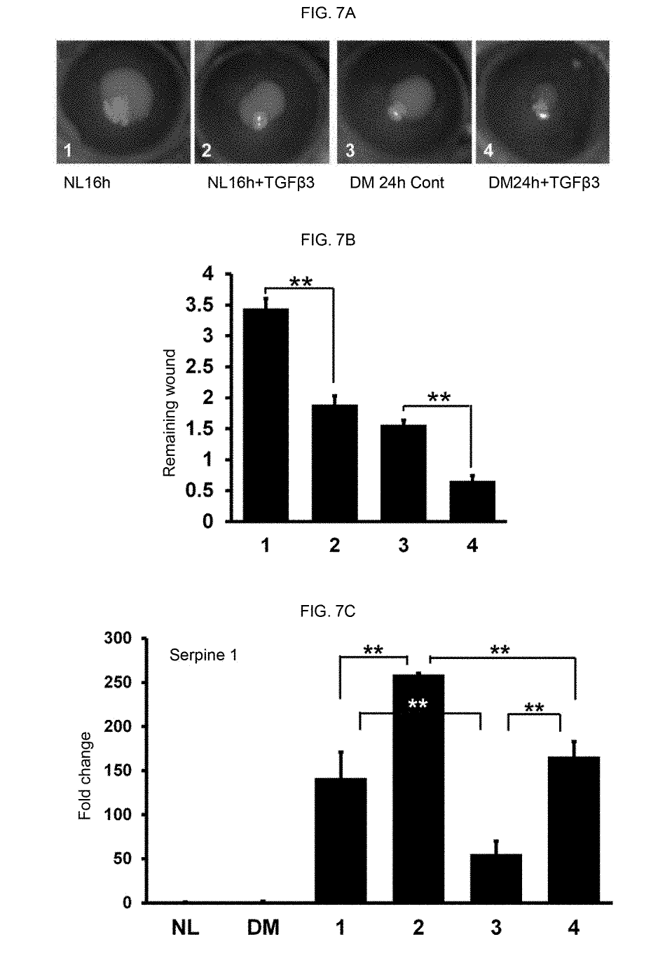

In STZ B6 mice, the addition of TGFβ significantly increased the rate of epithelial closure at 16 and 24 hpw (FIGS. 7A and B). Using Serpine 1, a common target of TGFβ (2, 3) −2 and −3) as an example, the data shows that its mRNA levels were correlated to the levels of TGFβ 3 activity and to that of the rates of wound closure in NL and DM mice (FIG. 7C).

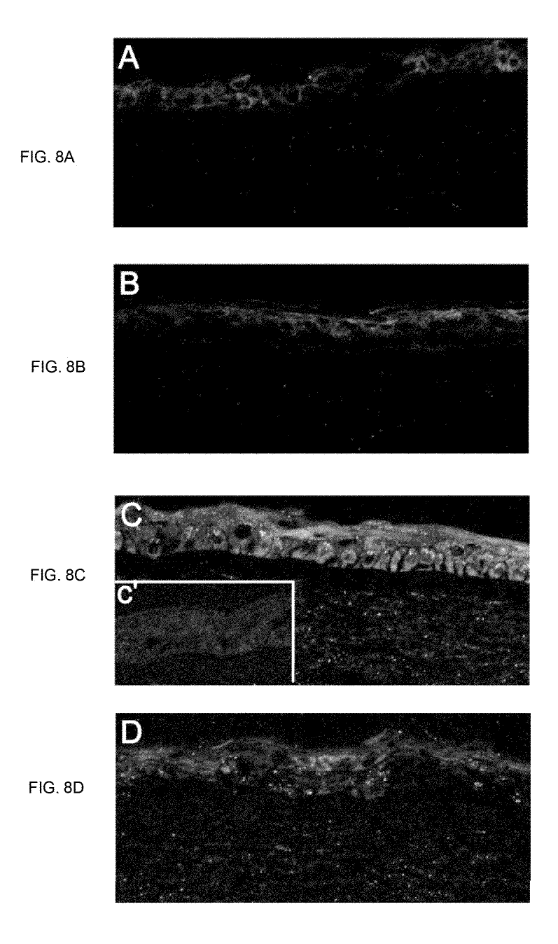

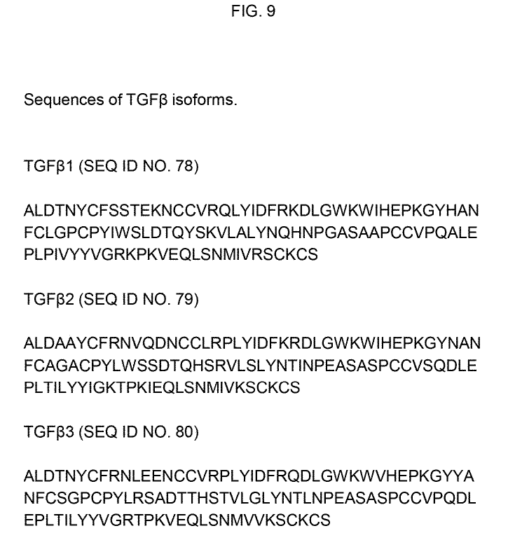

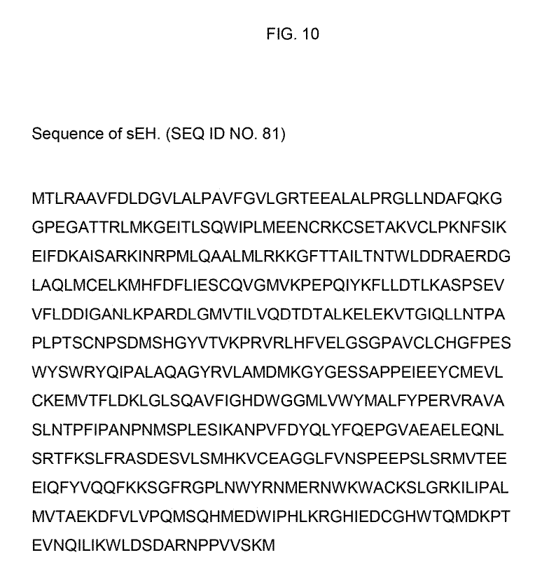

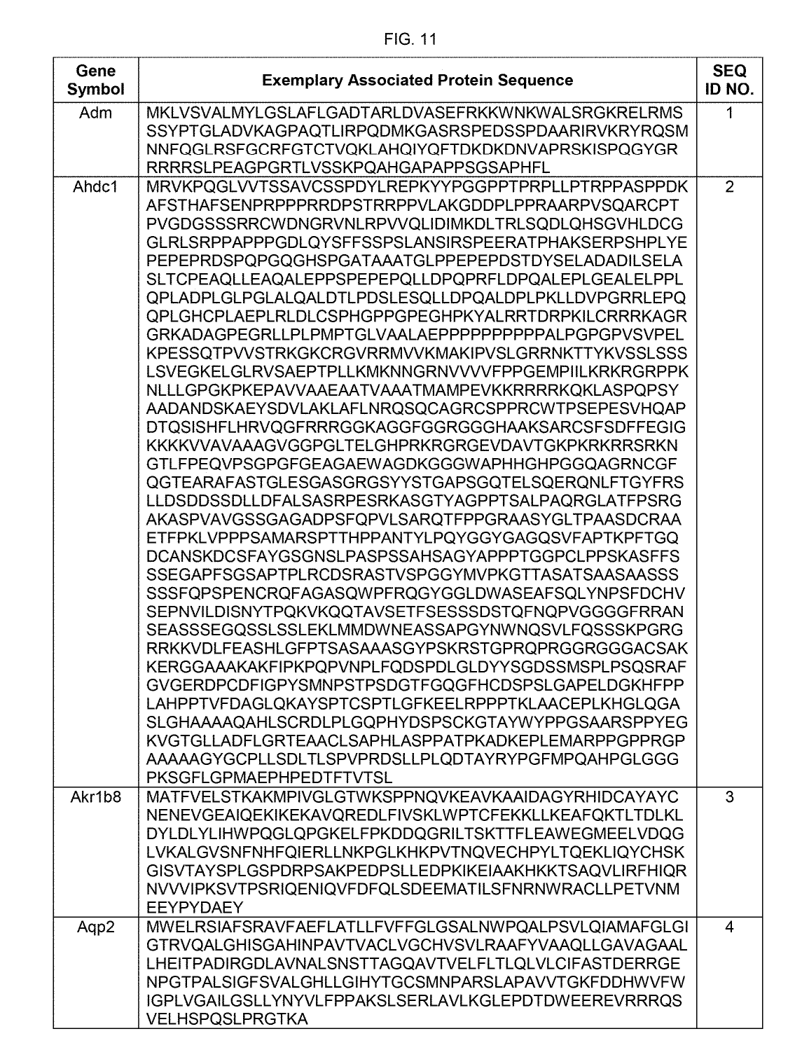

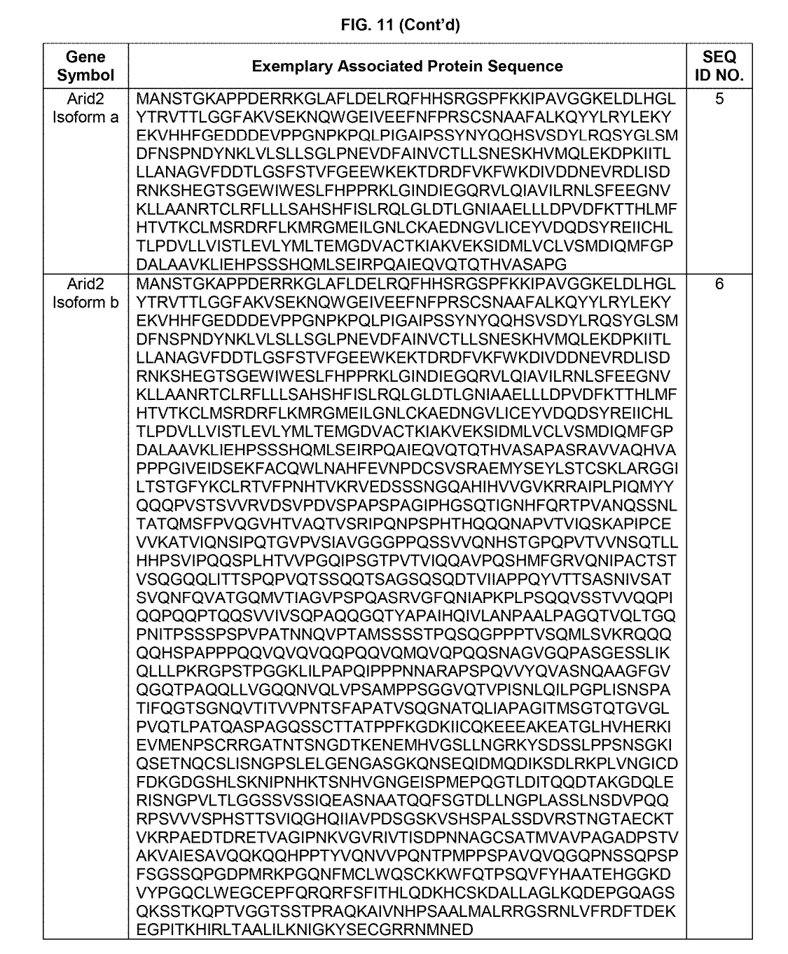

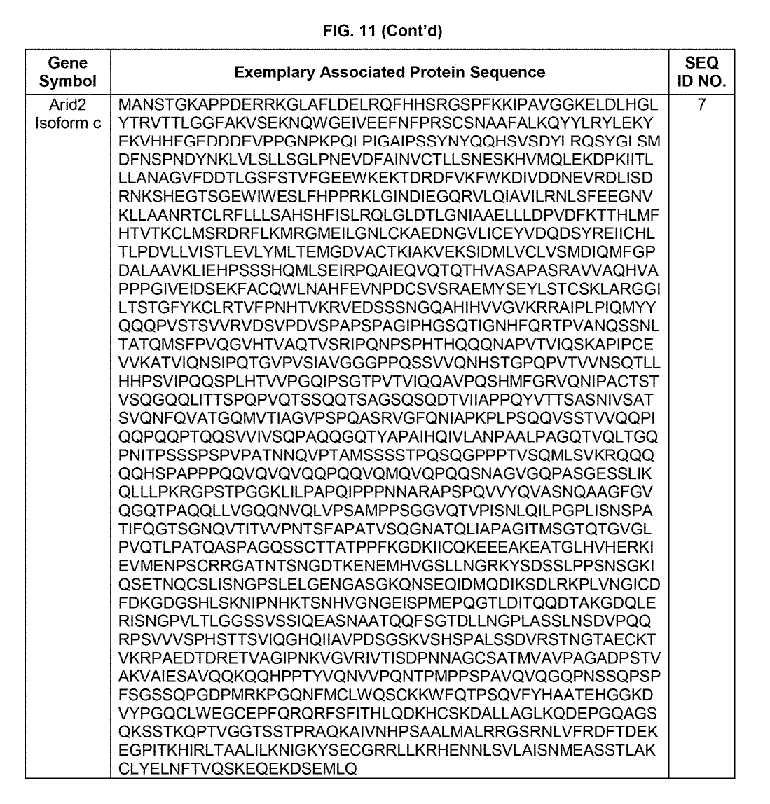

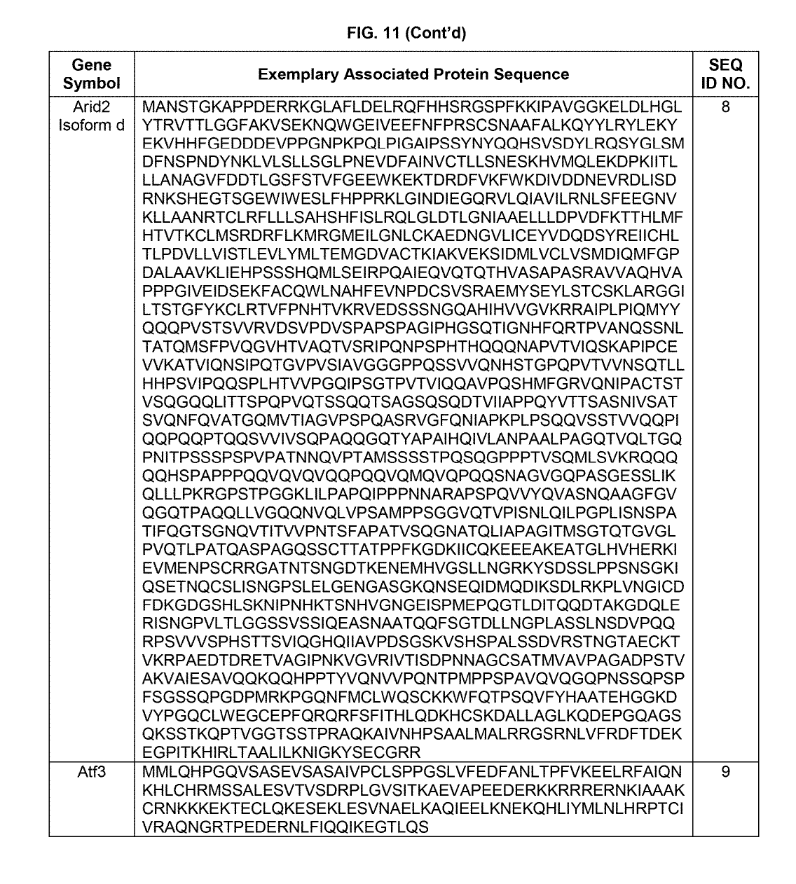

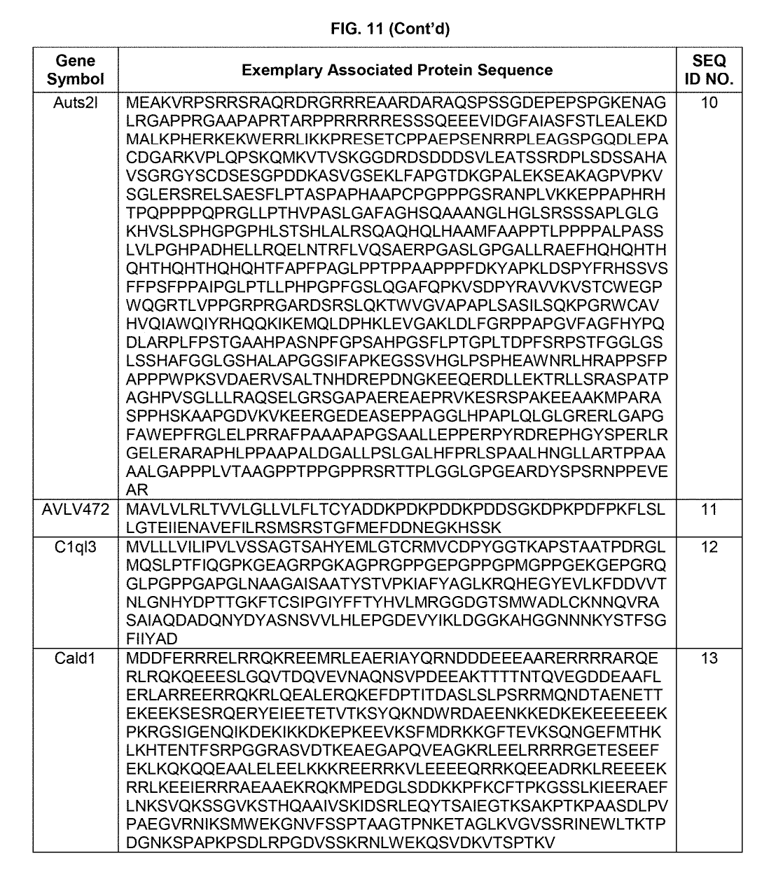

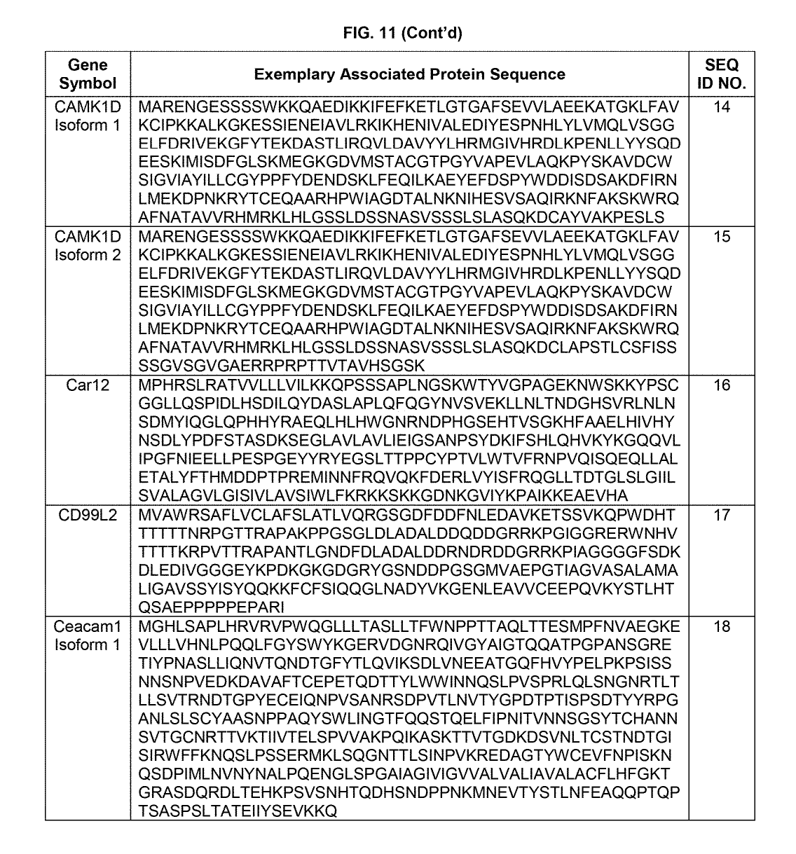

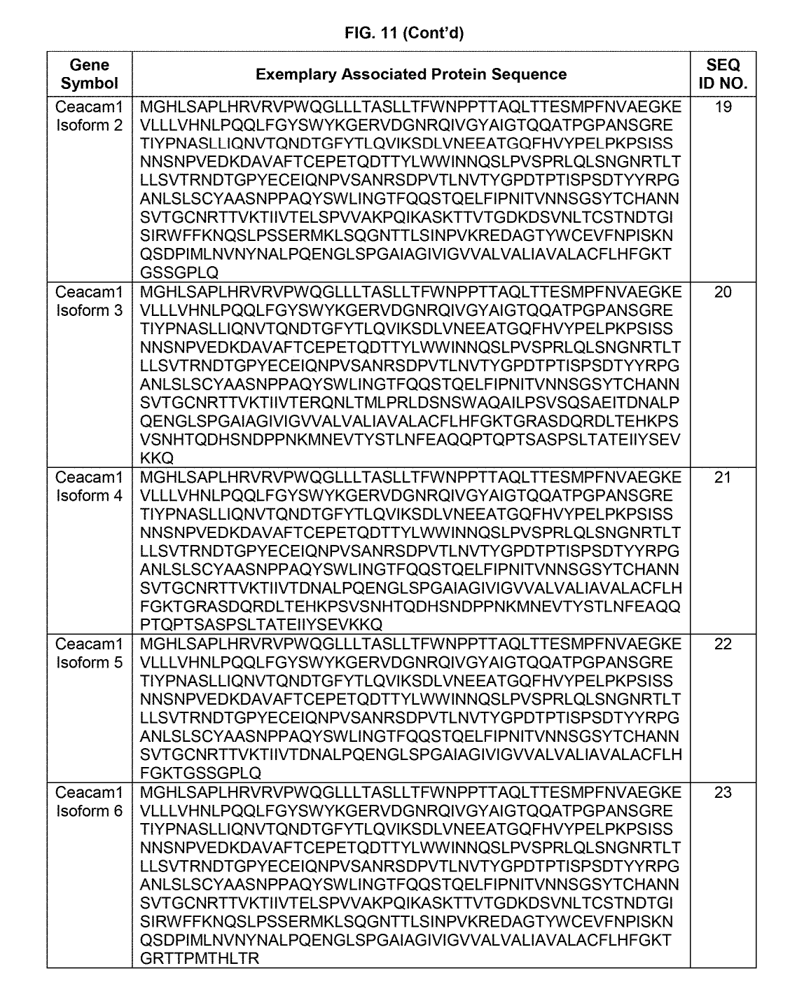

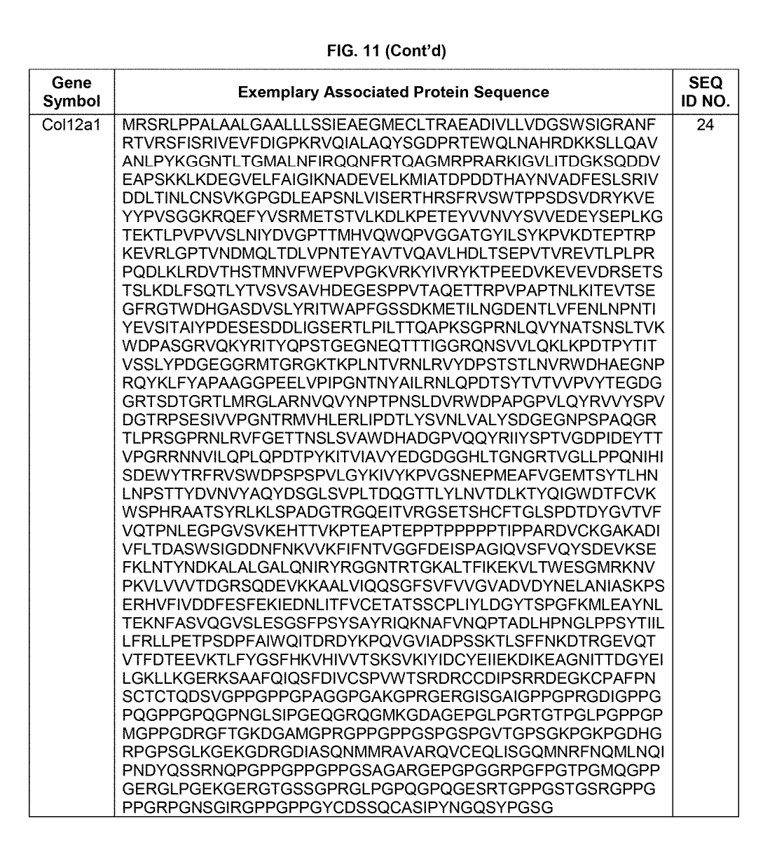

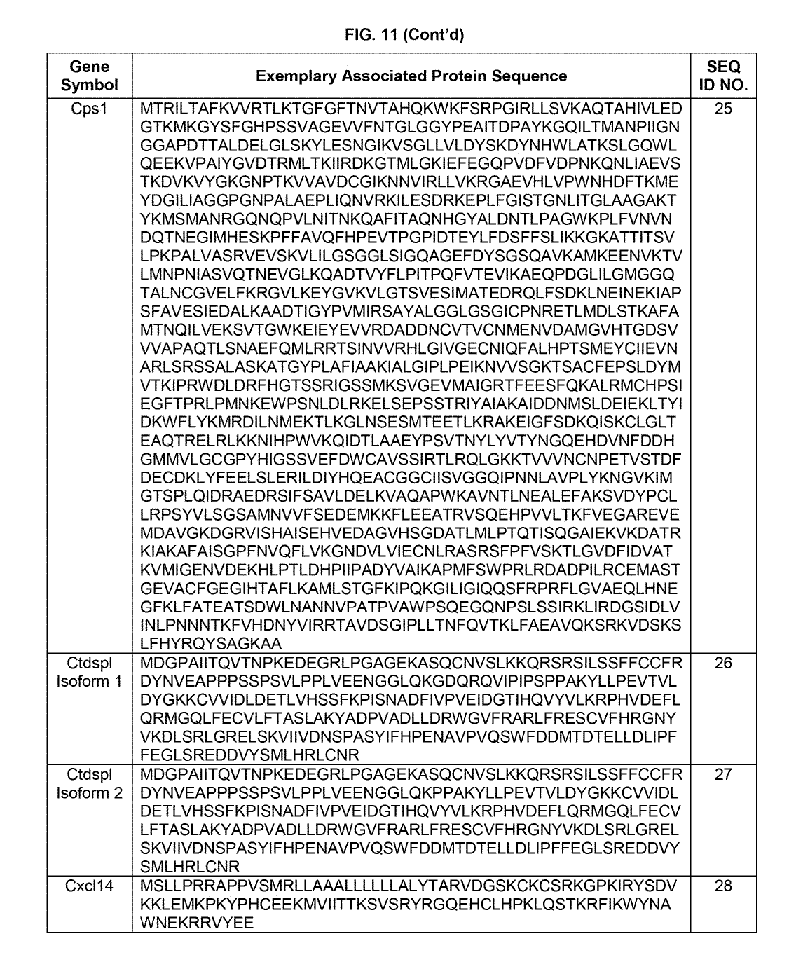

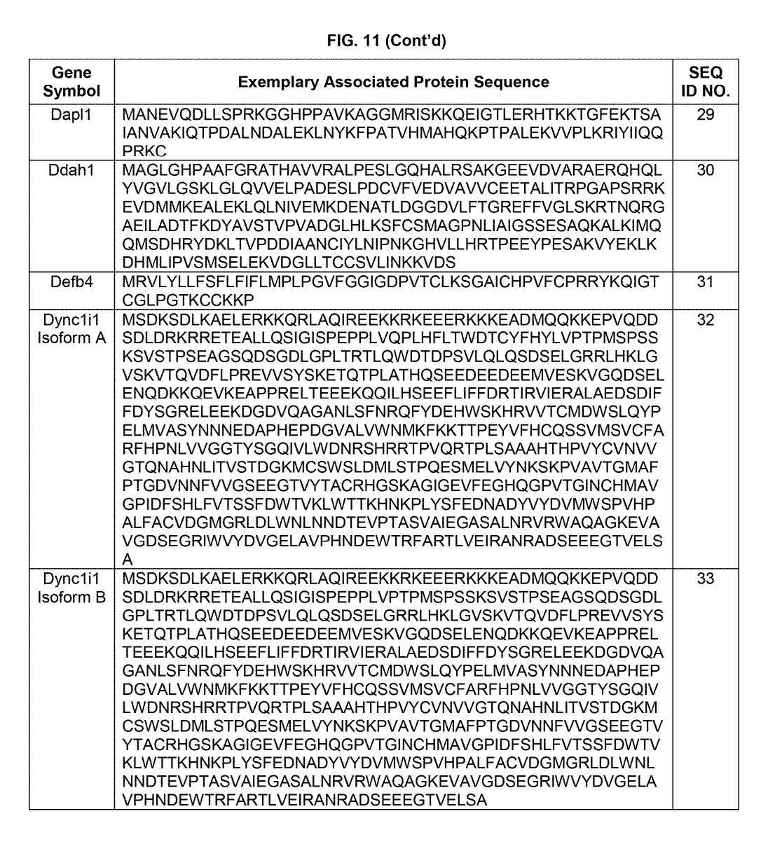

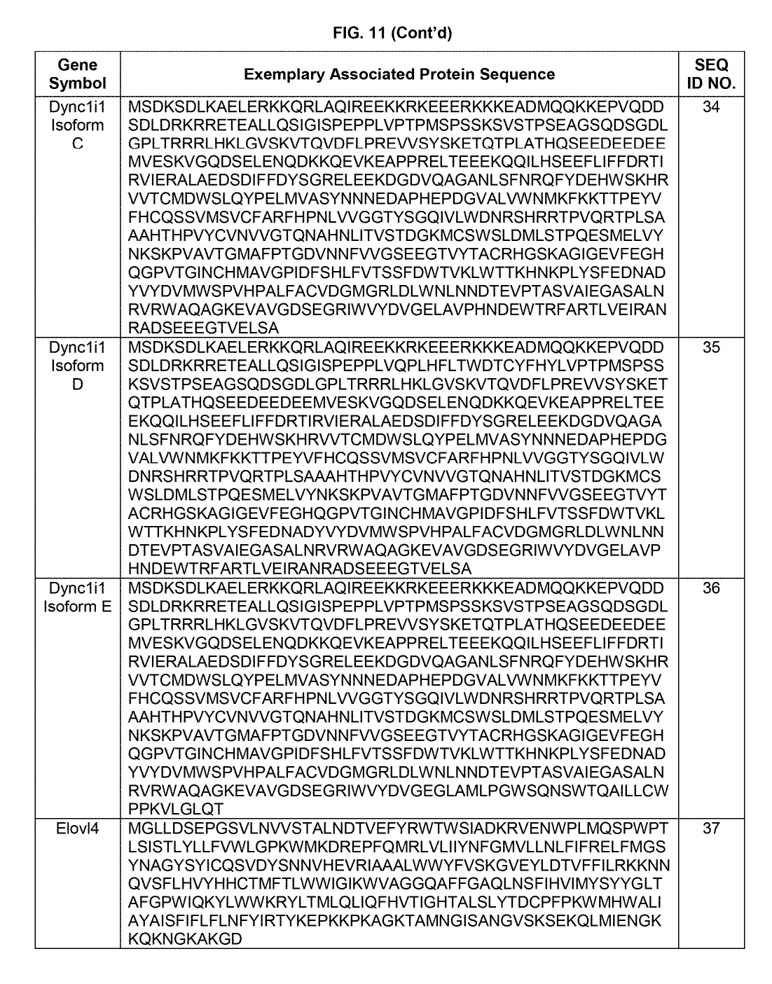

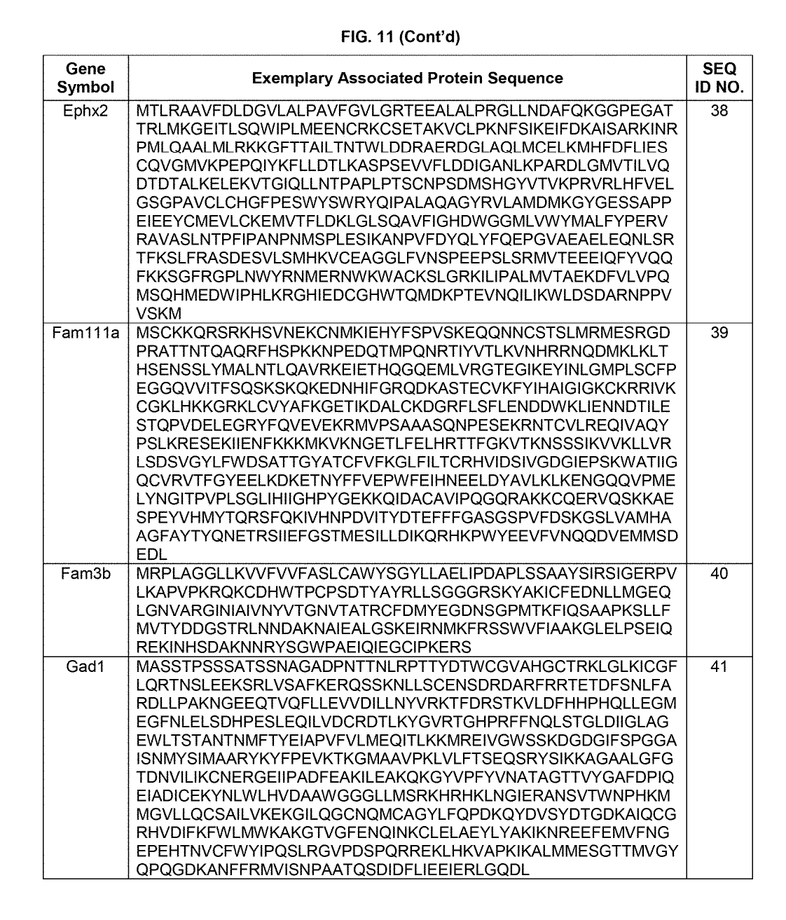

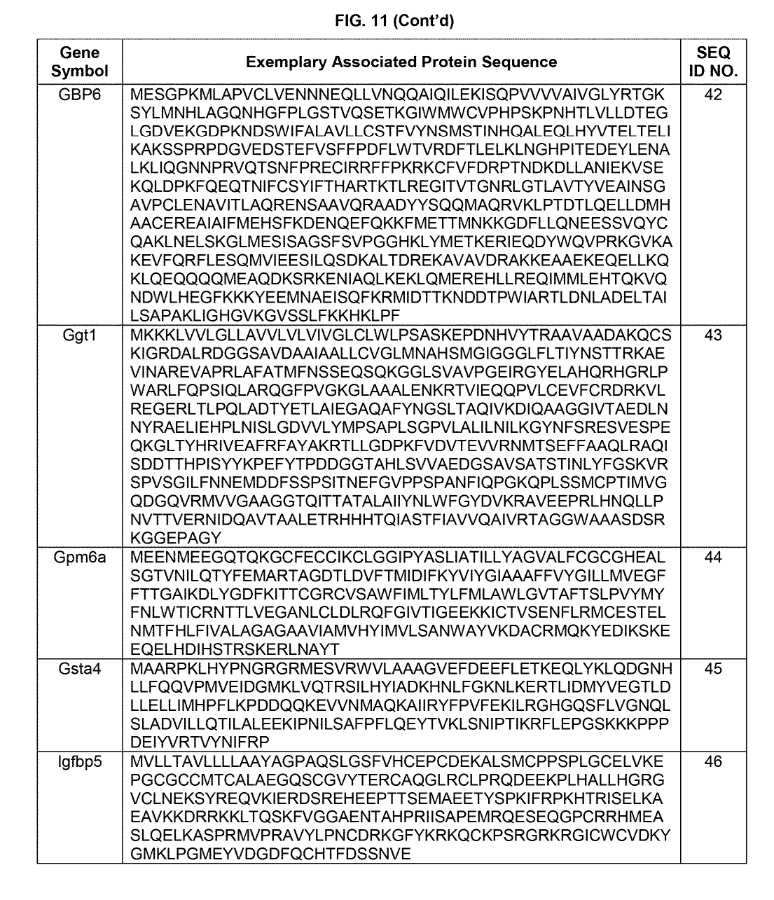

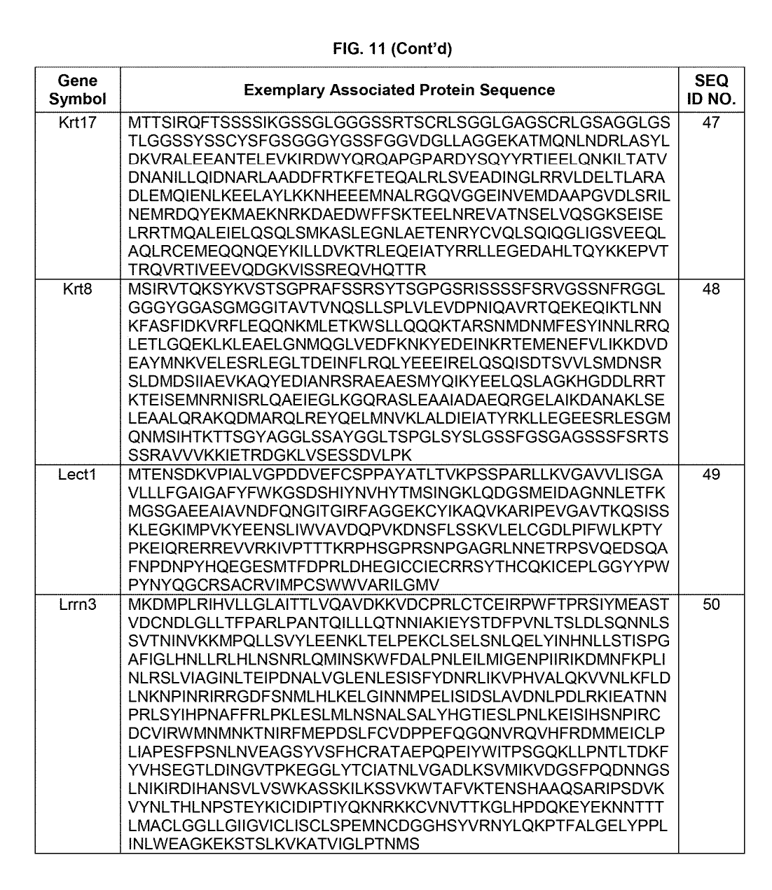

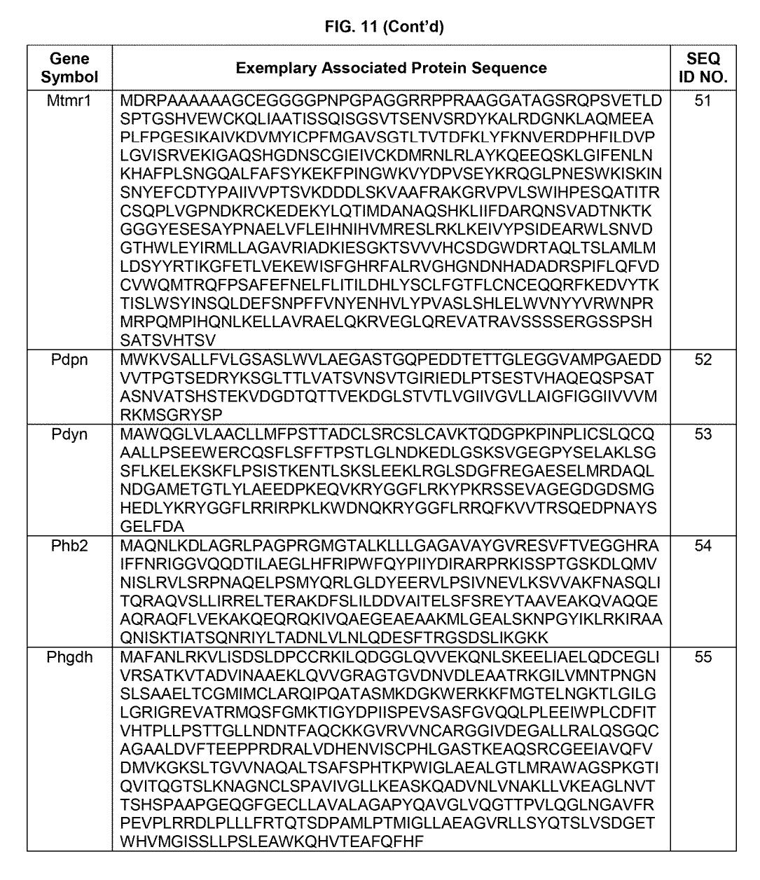

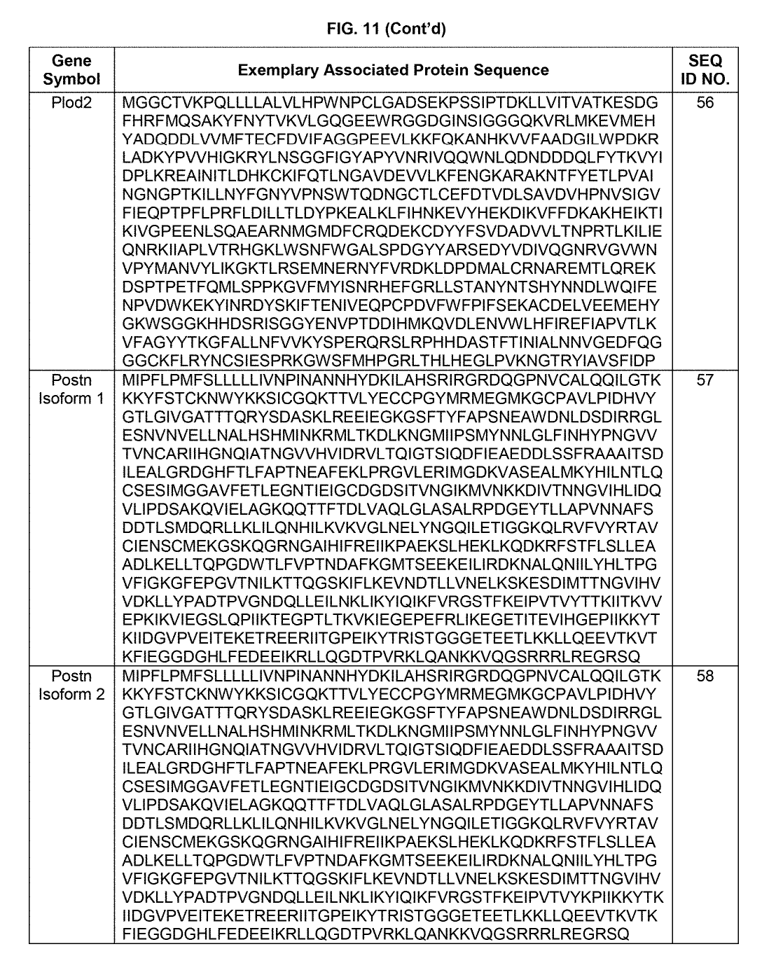

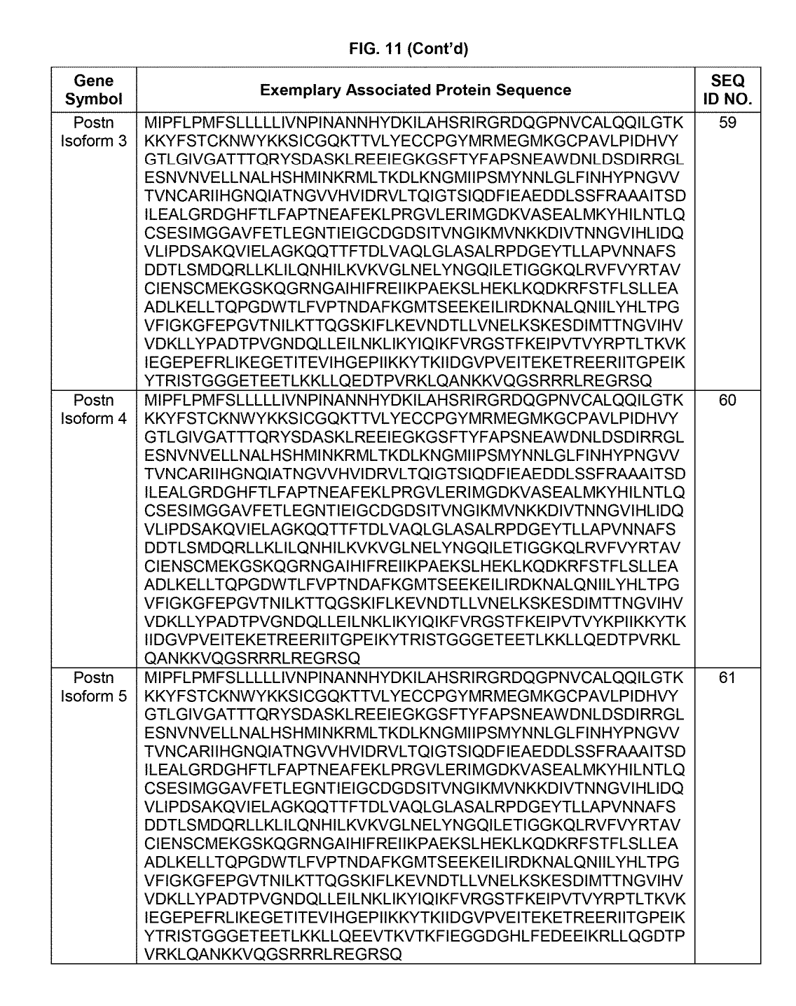

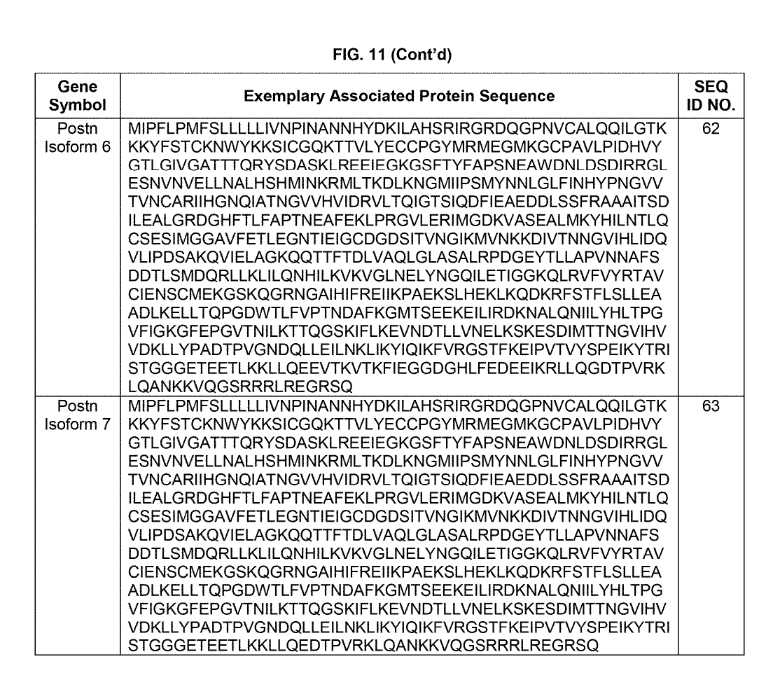

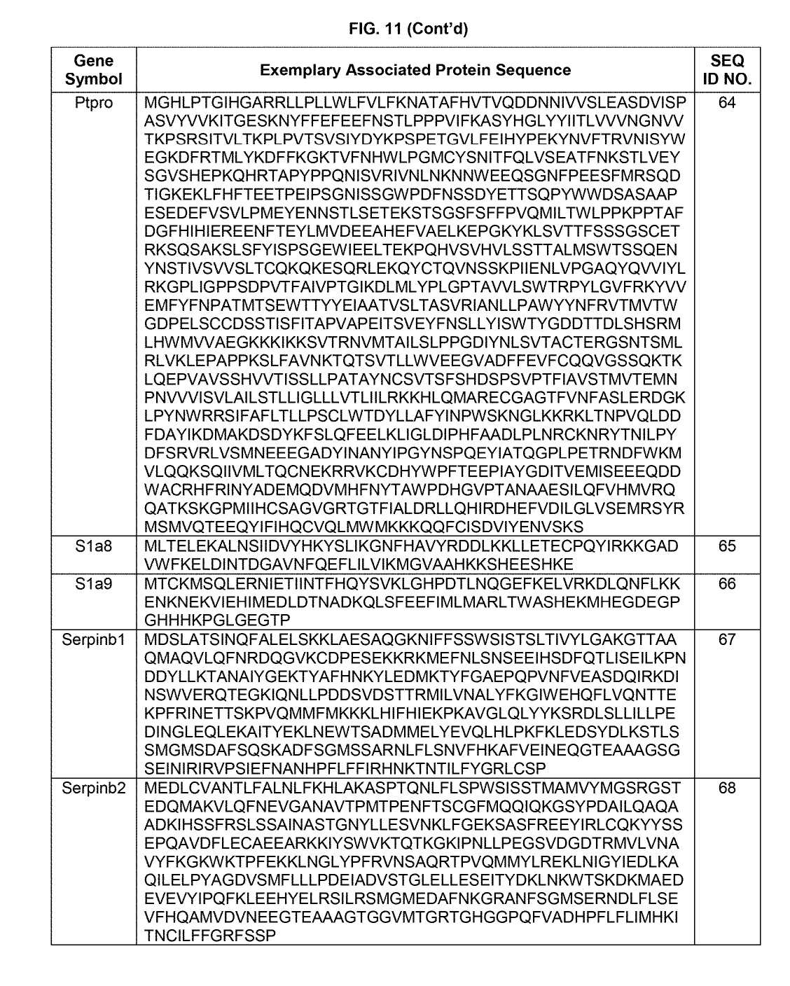

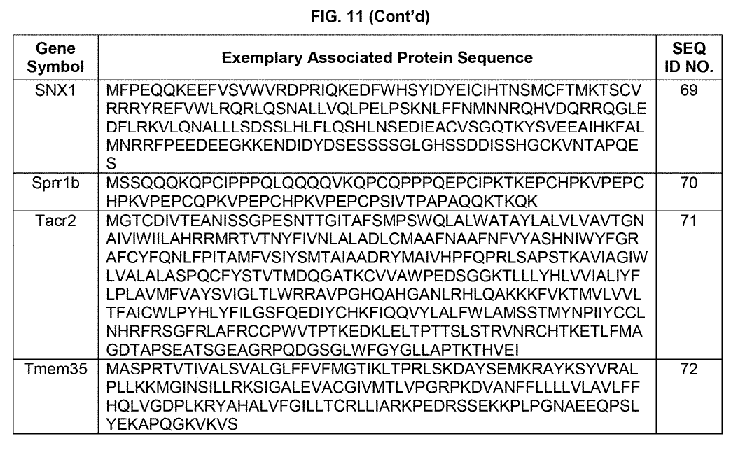

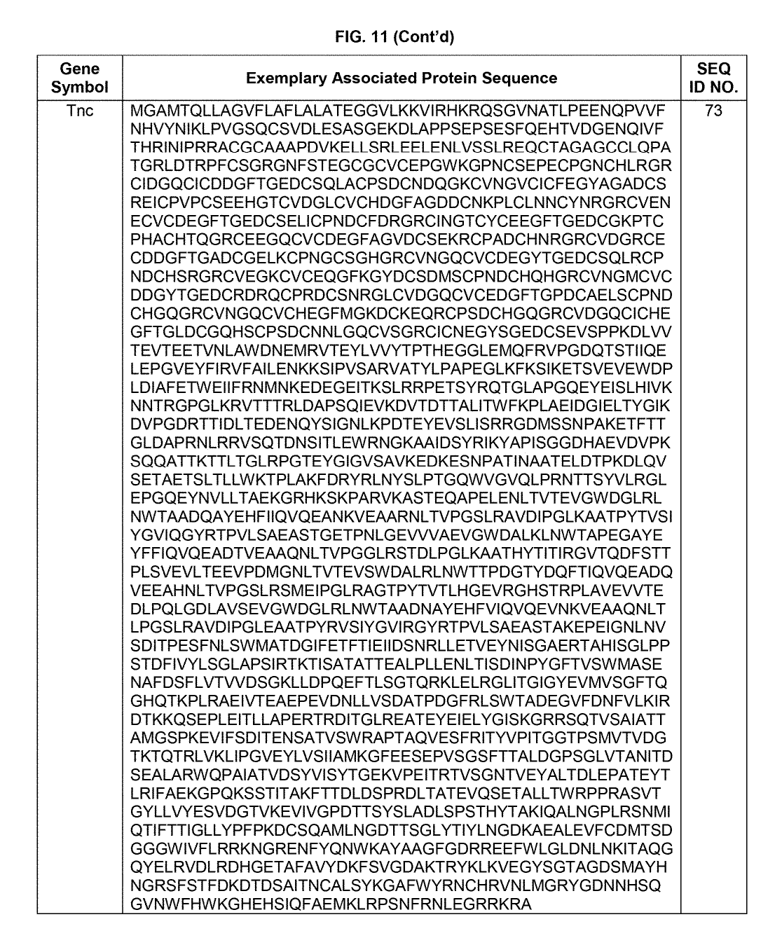

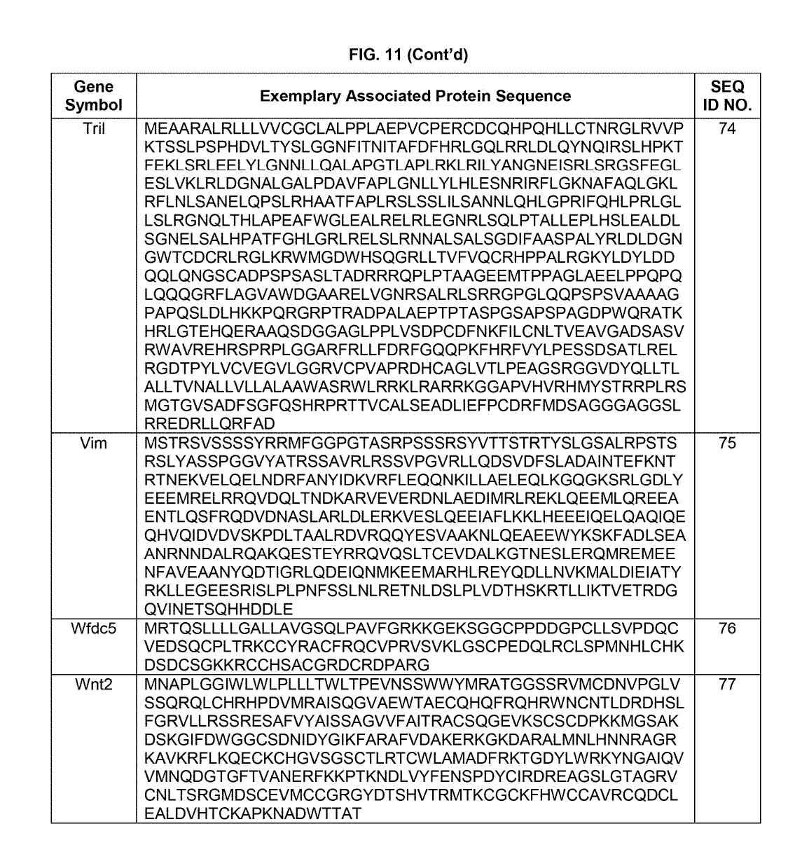

TGFβ 3 expression in cultured human UW and WH corneas. Delayed epithelial wound healing has also been shown in organ-cultured human corneas. NL and DM corneal cryostat sections without (direct embedded in OCT) or with epithelial wounding (which were allowed to heal in an organ culture setting (2)) were obtained. TGFβ3 expression in these corneas was assessed using confocal microscopy (FIGS. 8A-8D). In UW corneas, there were detectable levels of TGFβ3 in the epithelial layer. In the WH cornea with healed epithelial wound (2), all epithelial cell layers were positive with a strong TGFβ3 immunoreactivity in NL corneas, while weak staining was detected in DM CECs.

The genome-wide cDNA array analyses revealed a greatly altered expression of genes in response to wounding, and illustrated genes affected by hyperglycemia at a much larger scale in CECs. Unlike previous studies using limited arrays and RNA isolated from the cornea, the approach of the Examples used CECs thus avoiding false positive results from infiltration of immune cells in the stroma. Although the majority of the genes detected with differential expression are likely from epithelial cells, a small portion of differentially expressed genes could also be from dendritic cells, as they migrate with the healing epithelial sheets. Moreover, 8 weeks of hyperglycemia represented an early stage of DM when a significant delay of epithelial wound closure had begun. This allows characterization of early events of DM complication, which can be effectively intervened with or even reversed. With these advantages, the described genome-wide cDNA array revealed a large number of genes (probe sets) differentially expressed and the expression of many genes were verified by other means and/or consistent with their known function, further validating the value of the genome-wide microarray data.

The described genome-wide cDNA microarrays were used to compare the gene expression profiles of WH versus UW and DM versus NL CECs. The results revealed that wounding dramatically altered the expression of a large number of genes in both NL and DM corneas. Bioinformatics revealed many TGFβ-mediated signaling pathways and biological processes were altered. Using three DM mouse and rat models, it was shown that TGFβ3 activity is required for proper wound healing in NL corneas while exogenously added TGFβ3 promotes epithelial wound healing in the corneas of type 1 DM rats and mice and type 2 DM rats. Without being bound to a single theory, the effects of TGFβ s on epithelial wound closure are related to its ability to activate Smad-dependent and -independent pathways, to modulate its own expression, and to mediate target gene expression. Taken together, the results suggest that the hyperglycemia-suppressed genes such as TGFβ3 and Serpine 1 might be used as therapeutic reagents to accelerate delayed epithelial wound healing in DM corneas.

Comparison of WH DM with WH NL CECs revealed that 1888 probe sets had altered expression. Intriguingly, of the 36 probe sets with more than 3-fold increases in DM WH versus NL WH, only 4 are identified genes; the rest were derived from DNA sequence tags without gene identities. Among 4 genes, the up-regulation of Ephx2, which has been linked to subclinical cardiovascular disease in the Diabetes Heart Study, is interesting as it was shown to increase manifestations of DM nephropathy in mice (20). There were 17 genes with 4-fold decreases and among them, ATF3 and CEACAM1 have been shown to be associated with DM.

GO analyses revealed that the TGFβ-Smad mediated signaling pathway was impaired in DM WH CECs. Because TGFβ has been known to play a key role in corneal wound healing and fibrosis and the major cellular source of TGFβ in corneal wounds is the epithelium, the expression of 3 TGFβ isoforms was assessed. Hyperglycemia had no effects on TGFβ1, but greatly suppressed TGFβ 3 in DM WH CECs. In the cornea, earlier studies reported that both TGFβ 1 and β2 are seen within the corneal epithelium and stroma in the injured cornea while TGFβ 3 is not found in the anterior eye (for review see (31)).

More recent studies reported that TGFβ 3 was found in the basal cells of regenerating areas as well as in uninjured regions of the cornea after corneal injury (32). The described examples present strong evidence of up-regulation of TGFβ 3 expression in three animal models as well as in cultured human corneas. Hence, it was concluded that TGFβ 3 is expressed in CECs and its expression is up-regulated in response to wounding in NL corneas. More strikingly, almost total suppression of wound-induced TGFβ 3 up-regulation in DM corneas was shown, suggesting a unique role of TGFβ 3 in mediating epithelial wound closure.

Although TGFβ 1-3 share 60-80% identity, they are encoded by distinct genes, have different sequences in promoters, and exhibit different physiological and pathological activities in vivo (33). The importance of TGFβ in maintaining tissue homeostasis and in regulating wound healing in the cornea has been reported (for review see (31)). TGFβ's response to wounding can be mediated through Smad-dependent and independent signaling pathways in a cell type- and context-dependent manner (34-37). Among Smad-independent pathways, the PI3K/AKT pathway plays a significant role in regulating TGFβ-mediated responses (35).

Demonstration that wound-induced TGFβ3 expression in CECs is sensitive to hyperglycemia suggests a distinctive regulation and a unique role of the isoform in modulating epithelial wound healing. To define the role of identified genes, siRNA or recombinant TGFβ3 was administered to the corneas using subconjunctival injection, which is a common procedure performed at Ophthalmologists' office to deliver drugs to treat corneal diseases and glaucoma, and has been shown to allow bevacizumab to penetrate intact cornea (38-40). Using subconjunctival injection to deliver TGFβ3 siRNA in NL corneas and recombinant TGFβ3 in NL and DM corneas, TGFβ3 up-regulation in response to epithelial wounding contributing to epithelial wound healing and activating both canonical and non-canonical signaling pathways was demonstrated.

The altered expression of TGFβ3 was also confirmed in cultured human corneas post wound healing, indicating the relevance of the study. Moreover, it was demonstrated that exogenous TGFβ3 partially restores the healing rate of epithelial wounds in DM corneas of three rodent models, suggesting an important role for the isoform in mediating epithelial response to injury. Strikingly, we discovered that exogenously applied TGFβ3 elevated the levels of latent TGFβ3, but not TGFβ1 or β2, at the protein levels, suggesting an auto-regulated expression of TGFβ3 in the WH CECs, such as that observed in the mouse skin carcinogenesis model (41). While the mechanisms underlying hyperglycemia-suppressed wound-induced TGFβ3 expression is unclear, early inhibitory effects of hyperglycemia on TGFβ3 expression, which differs from that of TGFβ1 (42), may exacerbate the suppression of TGFβ3 up-regulation in response to wounding. Finally, we showed that the expression of Serpine 1, a well-known TGFβ target gene (43; 44), is correlated to the activity of TGFβ3 in WH CECs of both NL and DM corneas.

The discovery that TGFβ has a unique role in mediating corneal epithelial wound healing is of great significance since 1) the major cellular source of TGFβ in corneal wounds is the epithelium (30); 2) TGFβ3, in contrast to TGFβ1, is considered an anti-fibrotic (45); 3) TGFβ3 halts fibroblast migration and selectively promotes re-epithelialization in the skin wounds (46), indicating a critical role in skin wound healing; and 4) recombinant TGFβ3 has been undergoing phase 3 clinical trials as an anti-scarring agent for adult skin wounds (45; 47; 48). The trial was considered a failure due to increased angiogenesis in post-surgery skin (45). However, unlike the pathogenesis of DM retinopathy, nephropathy, and atherosclerotic plaque where there is excessive angiogenesis, blood vessel growth is impaired in DM wound healing (49). Hence, although TGFβ3 may potentially be used as a useful therapy to treat delayed wound healing or re-epithelialization in the cornea and in the skin for the benefit of accelerating wound healing and reducing inflammation that should outweigh adverse effects caused by neovascularization, which may not occur during DM wound healing or re-epithelialization (49).

### Example 2

A number of experiments are conducted to further define the role of up-regulation of TGFβ1, TGFβ2, TGFβ3, Serpine 1, fibronectin, lumican, epidermal growth factor ligands, activin and Nodal as well as the down-regulation of sEH/Ephx2 in the promotion of wound healing, promotion of re-epithelialization, reduction of the occurrence and/or severity of ulcers, preservation of nerve function and/or integrity, promotion of eye health and maintenance of vision. In the experiments, each experiment having relevant control conditions, each of the listed compounds is administered as part of a therapeutic composition (including as a direct therapeutic and/or as part of a genetic therapy as described herein). With the objective end points for each treatment outcome, the therapeutic compositions are shown to cause significantly significant improvements as follows:

| Compound | Treatment Outcome | Objective End Point |
|---|---|---|
| TGFβ1 | Promotes wound | Time to wound closure or |
| | healing | establishment of a biological barrier |
| | Promotes | Establishment of a biological barrier |
| | re-epithelialization | or Superficial Punctate Keratitis |
| | | (death of small groups of cells on |
| | | the surface of the cornea that can |
| | | be examined by a slit lamp) |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| TGFβ2 | Promotes wound | Time to wound closure or |
| | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| TGFβ3 | Promotes wound | Time to wound closure or |
| | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | clear and transparent cornea, no |
| | | fluorescence staining under a slit |
| | | lamp microscope |
| Serpine 1 | Promotes wound | Time to wound closure or |
| | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| Epidermal | Promotes wound | Time to wound closure or |
| growth | healing | establishment of a biological barrier |
| factor | Promotes | Establishment of a biological barrier |
| ligand | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| Activin | Promotes wound | Time to wound closure or |
| | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| Nodal | Promotes wound | Time to wound closure or |
| | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers, | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |
| sEH | Promotes wound | Time to wound closure or |
| inhibition | healing | establishment of a biological barrier |
| | Promotes | Establishment of a biological barrier |
| | re-epithelialization | or Superficial Punctate Keratitis |
| | Reduces occurrence | Slit lamp microscopy for surface |
| | and/or severity of | (ir)regularity |
| | ulcers | |
| | Preserves nerve | Corneal (or skin) sensitivity. |
| | function integrity | |
| | Preserves nerve | Confocal microscopy to determine |
| | integrity | the nerve fiber health (length, |
| | | diameters and branches) |
| | Promotes eye health | Epithelial integrity, ocular surface |
| | | regularity and/or normal tear |
| | | secretion. |
| | Maintains vision | Visual scores as determined by an |
| | | ophthalmologist |

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically significant reduction in an embodiment's ability to promote wound healing or re-epithelialization, reduce the negative effects of neuropathies and/or promote eye health and maintenance of vision in diabetics.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

1. Kaji Y: Prevention of diabetic keratopathy. Br J Ophthalmol 2005; 89:254-255
2. Kabosova A, Kramerov A A, Aoki A M, Murphy G, Zieske J D, Ljubimov A V: Human diabetic corneas preserve wound healing, basement membrane, integrin and MMP-10 differences from normal corneas in organ culture. Exp Eye Res 2003; 77:211-217
3. Zagon I S, Sassani J W, McLaughlin P J: Insulin treatment ameliorates impaired corneal reepithelialization in diabetic rats. Diabetes 2006; 55:1141-1147
4. Xu K, Yu F S: Impaired Epithelial Wound Healing and EGFR Signaling Pathways in the Corneas of Diabetic Rats. Invest Ophthalmol Vis Sci 2011
5. Inoue A, Watanabe T, Tominaga K, Tsugawa K, Nishio K, Takahashi K P, Kaneda K: Association of hnRNP 51 proteins with vimentin intermediate filaments in migrating cells. Journal of cell science 2005; 118:2303-2311
6. Wang F, Gao N, Yin J, Yu F S: Reduced innervation and delayed re-innervation after epithelial wounding in type 2 diabetic Goto-Kakizaki rats. The American journal of pathology 2012; 181:2058-2066
7. Yin J, Huang J, Chen C, Gao N, Wang F, Yu F S: Corneal complications in streptozocin-induced type I diabetic rats. Invest Ophthalmol Vis Sci 2011; 52:6589-6596
8. Schultz R O, Van Horn D L, Peters M A, Klewin K M, Schutten W H: Diabetic keratopathy. Trans Am Ophthalmol Soc 1981; 79:180-199
9. Pflugfelder S C: Is autologous serum a tonic for the ailing corneal epithelium? Am J Ophthalmol 2006; 142:316-317
10. Chi C, Trinkaus-Randall V: New insights in wound response and repair of epithelium. Journal of cellular physiology 2013; 228:925-929
11. Martin P: Wound healing—aiming for perfect skin regeneration. Science 1997; 276:75-81.
12. Bikbova G, Oshitari T, Tawada A, Yamamoto S: Corneal changes in diabetes mellitus. Current diabetes reviews 2012; 8:294-302
13. Xu K P, Li Y, Ljubimov A V, Yu F S: High glucose suppresses epidermal growth factor receptor/phosphatidylinositol 3-kinase/Akt signaling pathway and attenuates corneal epithelial wound healing. Diabetes 2009; 58:1077-1085
14. Wang F, Song Y L, Li D H, Li C X, Wang Y, Zhang N, Wang B G: Type 2 diabetes mellitus impairs bone healing of dental implants in GK rats. Diabetes research and clinical practice 2010; 88:e7-9
15. Gao N, Sang Yoon G, Liu X, Mi X, Chen W, Standiford T J, Yu F S: Genome-wide transcriptional analysis of differentially expressed genes in flagellin-pretreated mouse corneal epithelial cells in response to Pseudomonas aeruginosa: involvement of S100A8/A9. Mucosal immunology 2013
16. Trostrup H, Lundquist R, Christensen L H, Jorgensen L N, Karlsmark T, Haab B B, Agren M S: S100A8/A9 deficiency in nonhealing venous leg ulcers uncovered by multiplexed antibody microarray profiling. The British journal of dermatology 2011; 165:292-301
17. Schaerli P, Willimann K, Ebert L M, Walz A, Moser B: Cutaneous CXCL14 targets blood precursors to epidermal niches for Langerhans cell differentiation. Immunity 2005; 23:331-342
18. Meuter S, Moser B: Constitutive expression of CXCL14 in healthy human and murine epithelial tissues. Cytokine 2008; 44:248-255
19. Navarro A, Perez R E, Rezaiekhaligh M H, Mabry S M, Ekekezie, II: Polarized migration of lymphatic endothelial cells is critically dependent on podoplanin regulation of Cdc42. American journal of physiology Lung cellular and molecular physiology 2011; 300:L32-42
20. Elmarakby A A, Faulkner J, Al-Shabrawey M, Wang M H, Maddipati K R, Imig J D: Deletion of soluble epoxide hydrolase gene improves renal endothelial function and reduces renal inflammation and injury in streptozotocin-induced type 1 diabetes. American journal of physiology Regulatory, integrative and comparative physiology 2011; 301:R1307-1317
21. Bolliger M F, Martinelli D C, Sudhof T C: The cell-adhesion G protein-coupled receptor BAI3 is a high-affinity receptor for C1q-like proteins. Proceedings of the National Academy of Sciences of the United States of America 2011; 108:2534-2539
22. Khan K, Rudkin A, Parry D A, Burdon K P, McKibbin M, Logan C V, Abdelhamed Z I, Muecke J S, Fernandez-Fuentes N, Laurie K J, Shires M, Fogarty R, Carr I M, Poulter J A, Morgan J E, Mohamed M D, Jafri H, Raashid Y, Meng N, Piseth H, Toomes C, Casson R J, Taylor G R, Hammerton M, Sheridan E, Johnson C A, Inglehearn C F, Craig J E, Ali M: Homozygous mutations in PXDN cause congenital cataract, corneal opacity, and developmental glaucoma. American journal of human genetics 2011; 89:464-473
23. Kang Y, Chen C R, Massague J: A self-enabling TGFbeta response coupled to stress signaling: Smad engages stress response factor ATF3 for Id1 repression in epithelial cells. Molecular cell 2003; 11:915-926
24. Cao Z, Wu H K, Bruce A, Wollenberg K, Panjwani N: Detection of differentially expressed genes in healing mouse corneas, using cDNA microarrays. Investigative ophthalmology & visual science 2002; 43:2897-2904
25. Varela J C, Goldstein M H, Baker H V, Schultz G S: Microarray analysis of gene expression patterns during healing of rat corneas after excimer laser photorefractive keratectomy. Investigative ophthalmology & visual science 2002; 43:1772-1782
26. Gao N, Yin J, Yoon G S, Mi Q S, Yu F S: Dendritic cell-epithelium interplay is a determinant factor for corneal epithelial wound repair. The American journal of pathology 2011; 179:2243-2253
27. Burdon K P, Lehtinen A B, Langefeld C D, Carr J J, Rich S S, Freedman B I, Herrington D, Bowden D W: Genetic analysis of the soluble epoxide hydrolase gene, EPHX2, in subclinical cardiovascular disease in the Diabetes Heart Study. Diabetes & vascular disease research: official journal of the International Society of Diabetes and Vascular Disease 2008; 5:128-134
28. Kim J Y, Lee S H, Song E H, Park Y M, Lim J Y, Kim D J, Choi K H, Park S I, Gao B, Kim W H: A critical role of STAT1 in streptozotocin-induced diabetic liver injury in mice: controlled by ATF3. Cell Signal 2009; 21:1758-1767
29. Najjar S M: Regulation of insulin action by CEACAM1. Trends in endocrinology and metabolism: TEM 2002; 13:240-245
30. Stramer B M, Austin J S, Roberts A B, Fini M E: Selective reduction of fibrotic markers in repairing corneas of mice deficient in Smad3. Journal of cellular physiology 2005; 203:226-232
31. Tandon A, Tovey J C, Sharma A, Gupta R, Mohan R R: Role of transforming growth factor Beta in corneal function, biology and pathology. Current molecular medicine 2010; 10:565-578
32. Huh M I, Chang Y, Jung J C: Temporal and spatial distribution of TGF-beta isoforms and signaling intermediates in corneal regenerative wound repair. Histology and histopathology 2009; 24:1405-1416
33. Saika S: TGFbeta pathobiology in the eye. Lab Invest 2006; 86:106-115
34. Goc A, Choudhary M, Byzova T V, Somanath P R: TGFbeta- and bleomycin-induced extracellular matrix synthesis is mediated through Akt and mammalian target of rapamycin (mTOR). Journal of cellular physiology 2011; 226:3004-3013
35. Hong M, Wilkes M C, Penheiter S G, Gupta S K, Edens M, Leof E B: Non-Smad Transforming Growth Factor-p Signaling Regulated by Focal Adhesion Kinase Binding the p85 Subunit of Phosphatidylinositol 3-Kinase. Journal of Biological Chemistry 2011; 286:17841-17850
36. Lu L, Wang J, Zhang F, Chai Y, Brand D, Wang X, Horwitz D A, Shi W, Zheng S G: Role of SMAD and Non-SMAD Signals in the Development of Th17 and Regulatory T Cells. The Journal of Immunology 2010; 184:4295-4306
37. Holm T M, Habashi J P, Doyle J J, Bedja D, Chen Y, van Erp C, Lindsay M E, Kim D, Schoenhoff F, Cohn R D, Loeys B L, Thomas C J, Patnaik S, Marugan J J, Judge D P, Dietz H C: Noncanonical TGFβ Signaling Contributes to Aortic Aneurysm Progression in Marfan Syndrome Mice. Science 2011; 332:358-361
38. Chong R S, Su D H, Tsai A, Jiang Y, Htoon H M, Lamoureux E L, Aung T, Wong T T: Patient acceptance and attitude toward an alternative method of subconjunctival injection for the medical treatment of glaucoma. Journal of glaucoma 2013; 22:190-194
39. Erdurmus M, Totan Y: Subconjunctival bevacizumab for corneal neovascularization. Graefe's archive for clinical and experimental ophthalmology=Albrecht von Graefes Archiv fur klinische and experimentelle Ophthalmologie 2007; 245:1577-1579
40. Dastjerdi M H, Sadrai Z, Saban D R, Zhang Q, Dana R: Corneal penetration of topical and subconjunctival bevacizumab. Investigative ophthalmology & visual science 2011; 52:8718-8723
41. Rundhaug J E, Park J, Fischer S M: Uncoordinated regulation of mRNA expression of the three isoforms of transforming growth factor-beta in the mouse skin carcinogenesis model. Molecular carcinogenesis 1997; 18:115-126
42. Liu G, Ding W, Neiman J, Mulder K M: Requirement of Smad3 and CREB-1 in mediating transforming growth factor-beta (TGF beta) induction of TGF beta 3 secretion. The Journal of biological chemistry 2006; 281:29479-29490
43. Samarakoon R, Overstreet J M, Higgins S P, Higgins P J: TGF-beta1->SMAD/p53/USF2->PAI-1 transcriptional axis in ureteral obstruction-induced renal fibrosis. Cell and tissue research 2012; 347:117-128
44. Samarakoon R, Higgins P J: Integration of non-SMAD and SMAD signaling in TGF-beta1-induced plasminogen activator inhibitor type-1 gene expression in vascular smooth muscle cells. Thrombosis and haemostasis 2008; 100:976-983
45. Henderson J, Ferguson M W, Terenghi G: The reinnervation pattern of wounds and scars after treatment with transforming growth factor beta isoforms. Journal of plastic, reconstructive & aesthetic surgery: JPRAS 2012; 65:e80-86
46. Han A, Bandyopadhyay B, Jayaprakash P, Lua I, Sahu D, Chen M, Woodley D T, Li W: The anti-motility signaling mechanism of TGFbeta3 that controls cell traffic during skin wound healing. Biol Open 2012; 1:1169-1177
47. Laverty H G, Occleston N L, Johnson M, Little J, Jones R, Fairlamb D, Ferguson M W, O'Kane S: Effects of avotermin (transforming growth factor beta3) in a clinically relevant pig model of long, full-thickness incisional wounds. Journal of cutaneous medicine and surgery 2010; 14:223-232
48. Bush J, So K, Mason T, Occleston N L, O'Kane S, Ferguson M W: Therapies with emerging evidence of efficacy: avotermin for the improvement of scarring. Dermatol Res Pract 2010; 2010
49. Costa P Z, Soares R: Neovascularization in diabetes and its complications. Unraveling the angiogenic paradox. Life sciences 2013
50. Saghizadeh M, Kramerov A A, Tajbakhsh J, Aoki A M, Wang C, Chai N N, Ljubimova J Y, Sasaki T, Sosne G, Carlson M R, Nelson S F, Ljubimov A V: Proteinase and growth factor alterations revealed by gene microarray analysis of human diabetic corneas. Invest Ophthalmol Vis Sci 2005; 46:3604-3615

1-43. (canceled)

44. A method of detecting genes that are differentially expressed in epithelial cell samples from diabetic versus non-diabetic subjects comprising
obtaining an epithelial cell sample derived from a subject; and
detecting expression levels of the genes in the epithelial cell sample by contacting the epithelial cell sample with probes or primers specific for the genes and detecting binding between the genes and the primers or probes,
wherein the genes comprise (1) C1qI3, Fam111a, SNX10, Gpm6a and Wfdc5; (2) Ephx2, Akr1b8, ElovI4, Ctdspl, and Pdyn; (3) Vim, Atf3, Tmem35, Cald1 and Ceacam1; or (4) Ephx2, CD99I2, Arid2, Ctdspl and Gsta4,
thereby detecting genes that are differentially expressed in epithelial cell samples from diabetic versus non-diabetic subjects.

45. A method of claim 44 wherein the genes comprise C1qI3, Fam111a, SNX10, Gpm6a and Wfdc5; and the epithelial cell sample is derived from un-wounded tissue.

46. A method of claim 44 wherein the genes comprise Ephx2, Akr1b8, ElovI4, Ctdspl, and Pdyn; and the epithelial cell sample is derived from un-wounded tissue.

47. A method of claim 44 wherein the genes comprise (1) C1qI3, Fam111a, SNX10, Gpm6a and Wfdc5 and (2) Ephx2, Akr1b8, ElovI4, Ctdspl, and Pdyn; and the epithelial cell sample is derived from un-wounded tissue.

48. A method of claim 44 wherein the genes comprise Vim, Atf3, Tmem35, Cald1 and Ceacam1; and the epithelial cell sample is derived from wounded tissue.

49. A method of claim 44 wherein the genes comprise Ephx2, CD99I2, Arid2, Ctdspl and Gsta4; and the epithelial cell sample is derived from wounded tissue.

50. A method of claim 44 wherein the genes comprise (1) Vim, Atf3, Tmem35, Cald1 and Ceacam1 and (2) Ephx2, CD99I2, Arid2, Ctdspl and Gsta4; and the epithelial cell sample is derived from wounded tissue.

51. A method of claim 44 wherein the epithelial cell sample is derived from corneal tissue.

52. A method of claim 44 wherein the detecting binding occurs during or following a PCR assay.

53. A method of claim 44 wherein the detecting binding comprises detecting a label.

54. A method of claim 44 wherein the probes are present on a nucleic acid substrate array.

55. A method of claim 44 wherein the detecting binding occurs during or following an immunoassay.
